(19) United States
(12) Patent Application Publication
(10) Pub. No.: US 2024/0423800 A1
(43) Pub. Date: Dec. 26, 2024

(54) HEART VALVE REPAIR DEVICES AND DELIVERY DEVICES THEREFOR

(71) Applicant: EDWARDS LIFESCIENCES CORPORATION, Irvine, CA (US)

(72) Inventors: Lauren R. Freschauf, Mission Viejo, CA (US); Steven M. Ford, Laguna Beach, CA (US); Sergio Delgado, Irvine, CA (US); Munir Hicham Eltal, Irvine, CA (US); Grant Matthew Stearns, Costa Mesa, CA (US); Eric Michael Oberwise, Newport Beach, CA (US)

(21) Appl. No.: 18/821,207

(22) Filed: Aug. 30, 2024

Related U.S. Application Data
(63) Continuation of application No. PCT/US2023/015610, filed on Mar. 20, 2023.
(60) Provisional application No. 63/321,867, filed on Mar. 21, 2022.

(51) Int. Cl. A61F 2/24

(52) U.S. Cl. CPC A61F 2/246; A61F 2/2466; A61F 2220/0016

(57) ABSTRACT

A device or implant is configured to be positioned within a native heart valve to allow the native heart valve to form a more effective seal. The device or implant can include connectors that hold movable components of the device together. Components of the device or implant can include flex limiting portions. Paddle frames of the device or implant can be configured to be moved between wide and narrow configurations while substantially maintaining a position where valve leaflets are pressed together by the paddle frames. Paddle frames and/or connectors that widen and narrow the paddle frames can be tapered.

### RELATED APPLICATIONS

The present application is a continuation of Patent Cooperation Treaty application no. PCT/US2023/015610, filed on Mar. 20, 2023, which claims the benefit of U.S. Provisional Patent Application No. 63/321,867, filed on Mar. 21, 2022, which are incorporated herein by reference in their entireties for all purposes.

### BACKGROUND

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves may be surgically repaired or replaced during open heart surgery. However, open heart surgeries are highly invasive, and complications may occur. Transvascular techniques can be used to introduce and implant devices to treat a heart in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

### SUMMARY

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure may be included in the examples summarized here.

In some implementations, there is provided an implantable device or implant (e.g., implantable device, etc.) that is configured to be positioned within a native heart valve to allow the native heart valve to form a more effective seal.

In some implementations, an implantable device or implant includes an anchor portion. Each anchor includes a plurality of paddles that are each moveable between an open position and a closed position.

In some implementations, an implantable device for repairing a native valve of a heart includes an outer paddle, an inner paddle, a clasp, a paddle frame, and a clip/connector (e.g., a frame clip, a frame connector, a paddle frame clip, a paddle frame connector, a strain reducer, etc.).

In some implementations, the clip/connector couples the paddle frame to at least one of the outer paddle, the inner paddle, and the clasp.

In some implementations, the paddle frame comprises an outer frame portion and an inner frame portion.

In some implementations, the clip/connector is coupled to at least one of the outer frame portion and the inner frame portion.

In some implementations, the clip/connector comprises an aperture configured to connect to the paddle frame.

In some implementations, the clip/connector aperture houses a portion of the paddle frame.

In some implementations, the clip/connector aperture houses at least one of the outer frame portion and the inner frame portion of the paddle frame.

In some implementations, the clip/connector aperture comprises a square shaped cross section.

In some implementations, the clip/connector aperture comprises a cross sectional shape that is the same as the cross-sectional shape as at least one of the outer frame portion and the inner frame portion.

In some implementations, the clip/connector comprises a first aperture and a second aperture.

In some implementations, the first aperture is coupled to the inner paddle portion and the second aperture is coupled to the outer paddle portion.

In some implementations, the clip/connector comprises an open configuration and a closed configuration.

In some implementations, in the open position, a head portion of the clip/connector is open such that the aperture is accessible by way of a channel, and wherein in the closed configuration, the head portion is closed such that the aperture is not accessible.

In some implementations, at least one of the inner frame portion and the outer frame portion can be disposed through the channel and into the aperture.

In some implementations, the clip/connector comprises a head portion and a stem portion.

In some implementations, the head portion comprises one or engaging members.

In some implementations, the one or more engaging members engage at least one of the inner paddle and outer paddle.

In some implementations, the one or more engaging members are positioned at a distal end of the clip/connector.

In some implementations, the engaging members comprise a zig zag configuration.

In some implementations, the engaging members extend through and hook onto at least one of the outer paddle and the inner paddle.

In some implementations, the clip/connector is a first paddle frame clip/connector and the implantable device further comprises a second paddle frame clip/connector. In some implementations, the first paddle frame clip/connector is coupled to the outer paddle and the second paddle frame clip/connector is coupled to the inner paddle.

In some implementations, a system includes one of the implementations of an implantable device that includes a clip or other connector, such as a paddle frame clip or paddle frame connector, and a catheter. The implantable device is coupled to the catheter.

In some implementations, an implantable device for repairing a native valve of a heart includes an outer paddle, an inner paddle, a clasp, and a paddle frame. In some implementations, the paddle frame comprises a flex limiting portion.

In some implementations, the flex limiting portion includes flex limiting members that engage one another upon bending of the flex limiting portion to stop further bending of the flex limiting portion.

In an example implementation, the flex limiting portion comprises a clip/connector region disposed between a first flex limiting component and a second flex limiting component.

In some implementations, a flex limiting component comprises an L-shaped bend.

In some implementations, the clip/connector region comprises a rectangular, square, trapezoidal, or an irregularly shaped cross section.

In some implementations, a clip/connector (e.g., a paddle frame clip, connector, paddle frame connector, strain reducer, etc.) is coupled with the clip/connector region between the first flex limiting component and the second flex limiting component.

In some implementations, the clip/connector region comprises a width that is substantially the same as a width of the clip/connector.

In some implementations, a system includes one of the implementations of an implantable device that includes a paddle frame with a flex limiting portion. The implantable device is coupled to the catheter.

In some implementations, an implantable device for repairing a native valve of a heart includes a pair of paddle frames that are configured to move between a wide configuration and a narrow configuration.

In some implementations, the pair of paddle frames are moveable between an open position and a closed position where the paddle frames press leaflets of the native heart valve together.

In some implementations, the pair of paddle frames have a first paddle frame coaptation position where the native leaflets are first pressed together when the pair of paddle frames are in the closed position and are in the wide configuration.

In some implementations, the pair of paddle frames have a second paddle frame coaptation position where the native leaflets are first pressed together when the pair of paddle frames are in the closed position and are in a fifty percent narrowed configuration.

In some implementations, the second paddle frame coaptation position is within 2 mm of the first paddle frame coaptation position.

In some implementations the second paddle frame coaptation position is within 1 mm of the first paddle frame coaptation position.

In some implementations, the second paddle frame coaptation position is within 0.75 mm of the first paddle frame coaptation position.

In some implementations, the second paddle frame coaptation position is within 0.5 mm of the first paddle frame coaptation position.

In some implementations, the second paddle frame coaptation position is between 0.25 mm and 0.75 mm of the first paddle frame coaptation position.

In some implementations, a device for repairing a native valve of a heart includes a pair of paddle frames that includes a pair of inner paddle frame portions and a pair of adjustable width outer paddle frame portions. The pair of paddle frames are movable between an open position and a closed position.

In some implementations, the outer frame portions are configured to pinch together in the closed position.

In some implementations, the pair of inner frame portions do not pinch together in the closed position.

In some implementations, the pair of outer frame portions are shapeset to provide a pinch point.

In some implementations, the pair of outer frame portions are shapeset such that one or more portions of each of the pair of outer frame portions is biased past a center line CL of the device.

In some implementations, the system comprises the device coupled to one or more catheters.

In some implementations, a device for repairing a native valve of a heart includes a pair of paddle frames that are movable between a fully widened configuration and a fully narrowed configuration. In some implementations, the pair of paddle frames are movable between an open position and a closed position both in the fully widened configuration and in the fully narrowed configuration.

In some implementations, the pair of paddle frames have a first shoulder height when the pair of paddle frames are closed and are in the fully narrowed configuration. In some implementations, the pair of paddle frames have a second shoulder height when the pair of paddle frames are closed and are in the fully widened configuration. In some implementations, the first shoulder height is close to the second shoulder height.

In some implementations, first shoulder height is between 70% and 100% of the second shoulder height.

In some implementations, the first shoulder height is within 1 mm of the second shoulder height.

In some implementations, the outer frame portions are configured to pinch together in the closed position in both the fully narrowed configuration and the fully widened configuration.

In some implementations, each paddle frame of the pair of paddle frames comprises a fixed width inner frame portion and an adjustable width outer frame portion,

In some implementations, the adjustable width outer frame portion is movable between the fully narrowed configuration and fully widened configuration.

In some implementations, a system includes the valve repair device coupled to one or more catheter.

In some implementations, a device for repairing a native valve of a heart a pair of paddle frames. In some implementations, the pair of paddle frames includes a pair of inner paddle frame portions and a pair of adjustable width outer paddle frame portions. In some implementations, the pair of paddle frames are movable between a fully widened configuration and a fully narrowed configuration.

In some implementations, the pair of paddle frames are movable between an open position and a closed position both in the fully widened configuration and in the fully narrowed configuration.

In some implementations, each outer paddle frame portion wraps around a corresponding inner paddle frame portion when viewed from a proximal end, the pair of paddles are in the closed position, and/or the pair of outer paddle frame portions are in the fully widened configuration.

In some implementations, the device includes a pair of clasps disposed between the pair of inner paddle frame portions.

In some implementations, each clasp of the pair of clasps is disposed between sides of the inner paddle frame portions when viewed from a proximal side. In some implementations, sides of the outer frame portions are parallel to sides of the inner frame portions when viewed from a proximal end.

In some implementations, a distance between sides of the outer frame portions and the inner frame portions is between 0.01 mm and 0.5 mm when viewed from a proximal end.

In some implementations, the outer frame portions curve away from the inner frame portions within 0.5 mm of ends of the sides of the inner frame portion when viewed from the proximal end.

In some implementations, a system includes the valve repair device coupled to one or more catheters.

In some implementations, a device for repairing a native valve of a heart includes a pair of paddle frames that are movable between a fully widened configuration and a fully narrowed configuration. In some implementations, the pair of paddle frames are movable between an open position and a closed position both in the fully widened configuration and in the fully narrowed configuration.

In some implementations, the pair of paddle frames are tapered in a direction from a proximal end toward a distal end when viewed from the front, when the pair of paddle frames are closed, and/or when the pair of paddle frames are in the fully narrowed configuration.

In some implementations, the pair of paddle frames are tapered at an angle between 30 and 80 degrees.

In some implementations, a connector extends from the pair of paddle frames to a cap of the device.

In some implementations, the connector is tapered in the direction from the proximal end toward the distal end.

In some implementations, a taper angle of the connector is within 5 degrees of the angle of taper of the pair of paddle frames.

In some implementations, a taper of the connector is continuous with the taper of the pair of paddle frames.

In some implementations, a system includes the valve repair device coupled to one or more catheters.

In some implementations, a device for repairing a native valve of a heart includes a pair of inner paddle frames and a pair of adjustable width outer frame portions. The pair of inner paddle frame portions have a first width. In some implementations, the pair of adjustable width paddle frame portions are movable between a fully widened configuration and a fully narrowed configuration having a second width.

In some implementations, the pair of paddle frames are movable between an open position and a closed position. In some implementations, the second width is less than or equal to the first width.

In some implementations, the pair of adjustable width paddle frame portions wrap around the pair of inner paddle frame portions when viewed from a proximal end of the device.

In some implementations, the pair of adjustable width paddle frame portions are configured to pinch together when the pair of adjustable width paddle fame portions are in the closed position.

In some implementations, a system includes the valve repair device coupled to one or more catheters.

Any of the above systems, devices, etc. can be used in one or more method(s) performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the above methods can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of implementations of the present disclosure, a more particular description of the certain examples and implementations will be made by reference to various aspects of the appended drawings. These drawings depict only example implementations of the present disclosure and are therefore not to be considered limiting of the scope of the disclosure. Moreover, while the FIGS. can be drawn to scale for some examples, the FIGS. are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing mitral regurgitation;

FIG. 3A illustrates a side perspective view of a mitral valve;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8-14 show an example of an implantable device or implant, in various stages of deployment;

FIG. 15 shows an example of an implantable device or implant that is similar to the device illustrated by FIGS. 8-14, but where the paddles are independently controllable;

FIGS. 16-21 show the example implantable device or implant of FIGS. 8-14 being delivered and implanted within a native valve;

FIG. 22 shows a perspective view of an example implantable device or implant in a closed position;

FIG. 23 shows a perspective view of an example implantable device or implant in a closed position;

FIG. 24 illustrates an example valve repair device with paddles in an open position;

FIG. 25A illustrates another example valve repair device with paddles in a closed position;

FIG. 25B illustrates a top view of an example valve repair device;

FIG. 26 illustrates a perspective view of an example implantable device having paddles of adjustable widths;

FIG. 27 is a cross-section of the implantable device of FIG. 26 in which the implantable device is bisected;

FIG. 28 is another cross-section of the implantable device of FIG. 26 in which the implantable device is bisected along a plane perpendicular to the plane illustrated in FIG. 28;

FIG. 29 is a schematic illustration of an example implant catheter assembly coupled to an implantable device in which an actuation element is coupled to a paddle actuation control and to a driver head of the implantable device;

FIG. 30 is an illustration of the assembly of FIG. 29 with the implantable device rotated 90 degrees to show the paddle width adjustment element coupled to an inner end of the connector of the implantable device and coupled to a paddle width control;

FIG. 31 illustrates a perspective view of a clip/connector in an open position;

FIG. 32 illustrates a perspective view of a clip/connector in a closed position;

FIG. 32A illustrates a perspective view of a clip/connector;

FIGS. 33-35 illustrate an example implantable valve repair device in a closed position;

FIG. 33A illustrates the clip/connector of FIG. 32A attached to components of the example implantable valve repair device of FIGS. 33-35;

FIGS. 36A-36C illustrate an example clip/connector on an implantable valve repair device;

FIG. 37A illustrates an example of an implantable valve repair device with a clip/connector coupled to the inner paddle portion;

FIG. 37B illustrates an example of an implantable valve repair device with a clip/connector coupled to the clasp;

FIG. 38A illustrates an example implantable valve repair device;

FIG. 38B illustrates the example implantable valve repair device of FIG. 38A with a clip/connector coupled to the inner frame portion and outer frame portion;

FIG. 39 illustrates an example implantable valve repair device with a clip/connector coupled to the inner frame portion and outer frame portion;

FIG. 40 illustrates an example implantable valve repair device with a clip/connector coupled a paddle portion;

FIG. 41-42 illustrate example clips or connectors on an implantable valve repair device;

FIGS. 43A-43D illustrate an example outer frame portion with a flex limiting portion;

FIGS. 44-48 illustrate example flex limiting portions of a paddle frame portion;

FIG. 49A illustrates an example flex limiting portion of an outer portion in an open position;

FIG. 49B illustrates an example flex limiting portions of an outer frame portion in a closed position;

FIGS. 50A-50B illustrate an example outer frame portion in an expanded position;

FIGS. 51A-51B illustrate an example outer frame portion in a narrow position;

FIG. 52 illustrates an example implantable valve repair device;

FIGS. 53-54 illustrate an example implantable valve repair device in an expanded position;

FIGS. 55-56 illustrate an example implantable valve repair device in a narrow position;

FIG. 57 is a schematic illustration of an example valve repair device;

FIG. 58 is a schematic illustration of an example valve repair device;

FIG. 59 is a schematic top view of an example of outer paddle frame portions that wrap around inner paddle frame portions;

FIG. 60 is a schematic top view of FIG. 59 with paths of leaflets illustrated;

FIG. 61 is a schematic illustration of a valve repair device with tapered expandable paddles;

FIG. 62 is a partial side view of an example implantable valve repair device with an example of an outer frame portion disconnected from the connector;

FIG. 63 is a partial top view of the valve repair device illustrated by FIG. 62;

FIG. 64 is a perspective view of the outer frame portion of the device of FIGS. 62 and 63;

FIG. 65 is a top view of the outer frame portion of the device of FIGS. 62 and 63;

FIG. 66 is a front view of the outer frame portion of the device of FIGS. 62 and 63;

FIG. 67 is side view of the outer frame portion of the device of FIGS. 62 and 63;

FIG. 68 is a partial perspective view of the device of the device of FIGS. 62 and 63;

FIG. 69 is a top view of components of an example valve repair device with expandable paddle frame portions that pinch together;

FIG. 70 is a side view of the components of the valve repair device of FIG. 69;

FIG. 71 is a front view of the components of the valve repair device of FIG. 69;

FIG. 72 is a top view of components of the valve repair device of FIG. 69 with another outer frame implementation illustrated;

FIG. 73 is a front view of components of the valve repair device of FIG. 63 with the outer frame implementation of FIG. 69 illustrated;

FIG. 74 is a side view of an example implantable valve repair device illustrating an outer paddle frame portion with an extended height;

FIG. 75 is a front view of an example valve repair device with tapered expandable paddles;

FIG. 76 is a perspective view of the valve repair device of FIG. 75;

FIG. 77 is a partial front view of a valve repair device with a narrowed outer paddle frame portion that is wider than the inner paddle frame portion; and

FIG. 78 is a partial front view of a valve repair device with a narrowed outer paddle frame portion that is wider than the inner paddle frame portion.

### DETAILED DESCRIPTION

The following description refers to the accompanying drawings, which illustrate example implementations of the present disclosure. Other implementations having different structures and operation do not depart from the scope of the present disclosure.

Example implementations of the present disclosure are directed to systems, devices, methods, etc. for repairing a defective heart valve. For example, various implementations of valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible. Further, the treatment techniques, methods, operations, steps, etc. described or suggested herein can be performed on a living subject (e.g., human, other animal, etc.) or on a non-living simulation, such as a cadaver, cadaver heart, simulator, imaginary person, etc.). When performed on a simulation, the body parts, e.g., heart, tissue, valve, etc., can optionally be referred to as “simulated” (e.g., simulated heart, simulated tissue, simulated valve, etc.) and can comprise, for example, computerized and/or physical representations of body parts, tissue, etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of). As described herein, reference to “simulation” encompasses use on a cadaver, computer simulator, imaginary person (e.g., if they are just demonstrating in the air on an imaginary heart), etc.

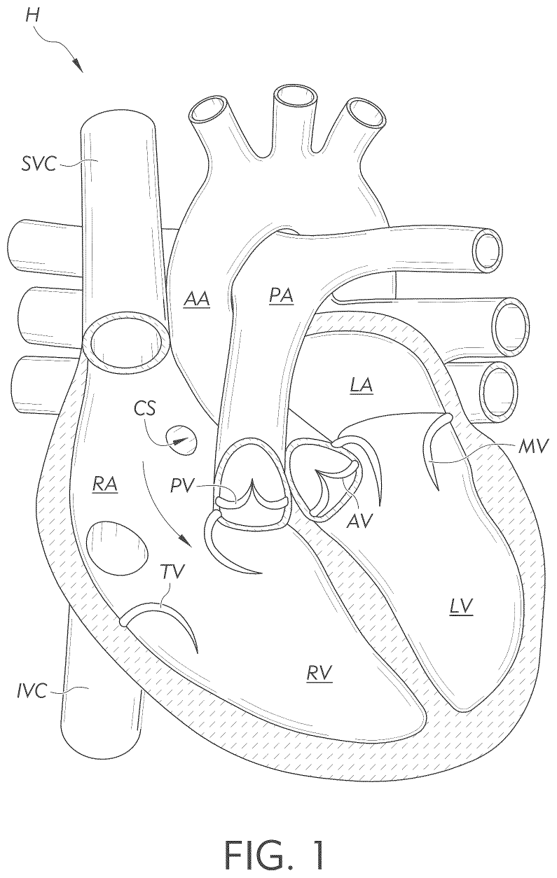

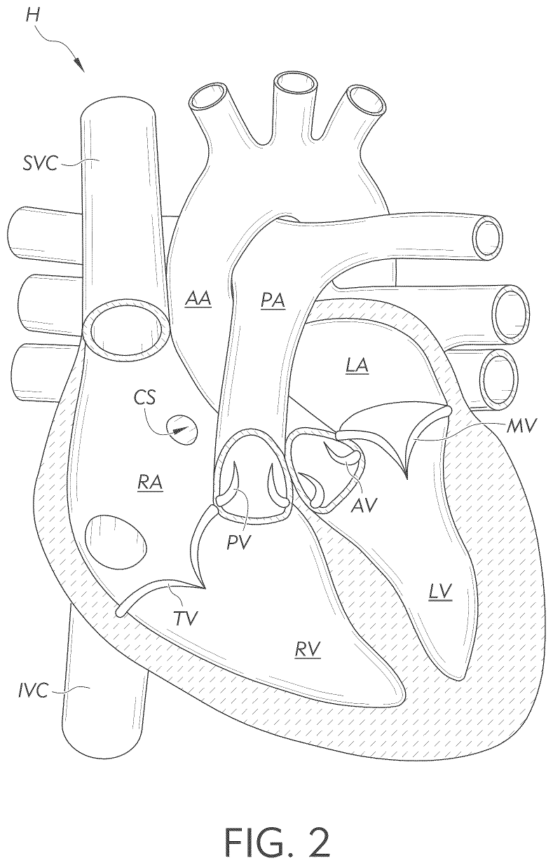

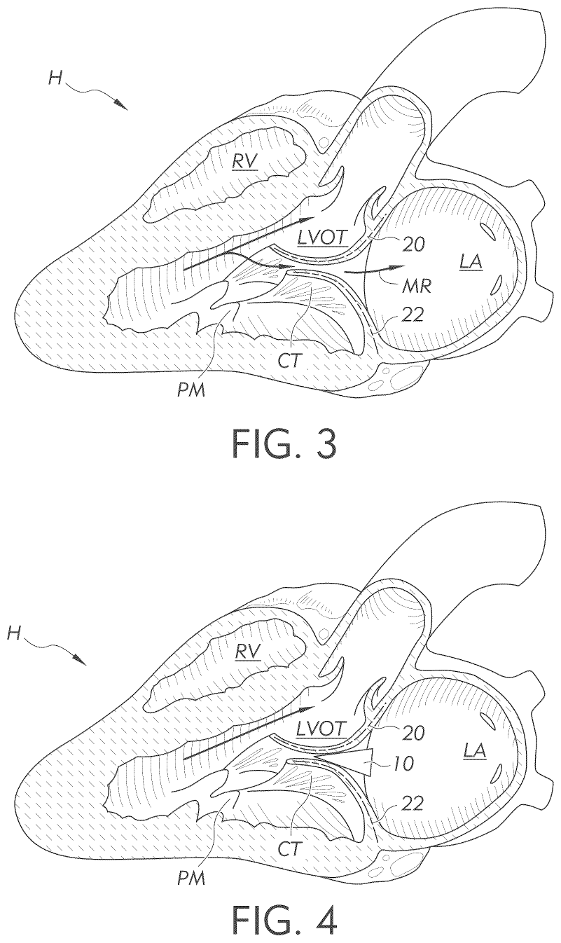

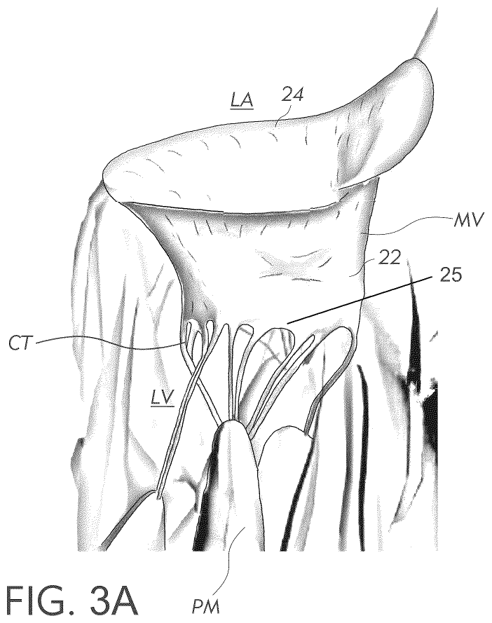

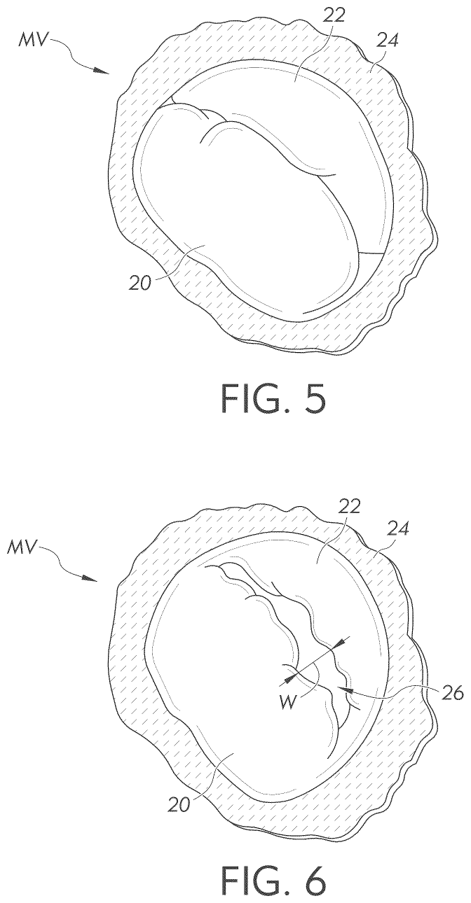

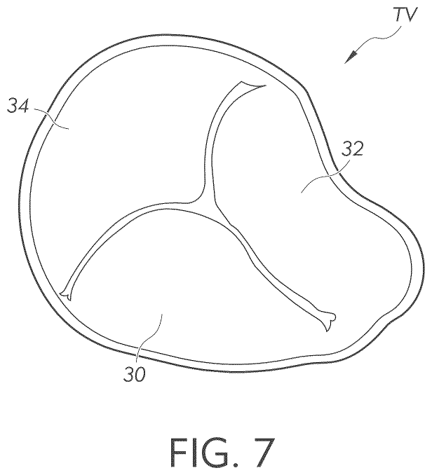

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent, inhibit or reduce blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

Referring now to FIGS. 1-7, the mitral valve MV includes two leaflets, the anterior leaflet 20 and the posterior leaflet 22. The mitral valve MV also includes an annulus 24 (see FIG. 5), which is a variably dense fibrous ring of tissues that encircles the leaflets 20, 22. As can be seen in FIG. 3A, the leaflets 20, 22 (leaflet 20 is behind leaflet 22 in FIG. 3A) are wider at the annulus 24 than at a free or movable edge 25. As such, there is a taper of the leaflets 20, 22 between the annulus 24 and the free or movable edge 25. As can also be seen in FIG. 3A, spacings between connections of the chordae tendineae CY to the free or movable edge 25 of the leaflets 20, 22 are small. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. As can also be seen in FIG. 3A, spacings between connections of the chordae tendineae CY to the free or movable edge 25 of the leaflets 20, 22 are small, since multiple chordae tendineae CT connect to the free or movable edge 25.

The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) may distort a native valve's geometry, which may cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant—or incompetent—which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction may be caused by rheumatic disease or dilation of a ventricle.

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 can have a width W between about 2.5 mm and about 17.5 mm, between about 5 mm and about 15 mm, between about 7.5 mm and about 12.5 mm, or about 10 mm. In some situations, the gap 26 can have a width W greater than 15 mm or even 17.5 mm. As set forth above, there are several different ways that a leaflet (e.g., leaflets 20, 22 of mitral valve MV) may malfunction which can thereby lead to valvular regurgitation.

In any of the above-mentioned situations, a valve repair device or implant is desired that is capable of engaging the anterior leaflet 20 and the posterior leaflet 22 to close the gap 26 and prevent or inhibit regurgitation of blood through the mitral valve MV. As can be seen in FIG. 4, an abstract representation of an implantable device, valve repair device, or implant 10 is shown implanted between the leaflets 20, 22 such that regurgitation does not occur during systole (compare FIG. 3 with FIG. 4). In some implementations, the coaptation element (e.g., spacer, coaption element, gap filler, etc.) of the device 10 has a generally tapered or triangular shape that naturally adapts to the native valve geometry and to its expanding leaflet nature (toward the annulus). In this application, the terms spacer, coaption element, coaptation element, and gap filler are used interchangeably and refer to an element that fills a portion of the space between native valve leaflets and/or that is configured such that the native valve leaflets engage or “coapt” against (e.g., such that the native leaflets coapt against the coaption element, coaptation element, spacer, etc. instead of only against one another).

Although stenosis or regurgitation may affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves may either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, may prevent the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation may occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the valve repair devices or implants provided herein can be centrally located between the three leaflets 30, 32, 34.

An example implantable device or implant can optionally have a coaptation element (e.g., spacer, coaption element, gap filler, etc.) and at least one anchor (e.g., one, two, three, or more). In some implementations, an implantable device or implant can have any combination or sub-combination of the features disclosed herein without a coaptation element. When included, the coaptation element (e.g., spacer, coaption element, gap filler, etc.) is configured to be positioned within the native heart valve orifice to help fill the space between the leaflets and form a more effective seal, thereby reducing or preventing or inhibiting regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device or implant can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral leaflets 20, 22 or tricuspid leaflets 30, 32, 34) that do not close completely.

The optional coaptation element (e.g., spacer, coaptation element, gap filler, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surfaces extend between the native tricuspid leaflets.

In some implementations, the anchor can be configured to secure the device to one or both of the native leaflets such that the coaptation element is positioned between the two native leaflets. In some implementations configured for use in the tricuspid valve, the anchor is configured to secure the device to one, two, or three of the tricuspid leaflets such that the coaptation element is positioned between the three native leaflets. In some implementations, the anchor can attach to the coaptation element at a location adjacent the ventricular portion of the coaptation element. In some implementations, the anchor can attach to an actuation element (e.g., an actuation shaft, actuation tube, actuation wire, etc.) to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element (e.g., shaft, actuation wire, etc.). The anchor can be configured to be positioned behind a native leaflet when implanted such that the leaflet is grasped by the anchor.

The device or implant can be configured to be implanted via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation methods for various implementations can be different and are more fully discussed below with respect to each implementation. Additional information regarding these and other delivery methods can be found in U.S. Pat. No. 8,449,599 and U.S. Patent Application Publication Nos. 2014/0222136, 2014/0067052, 2016/0331523, and PCT patent application publication Nos. WO2020/076898, each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

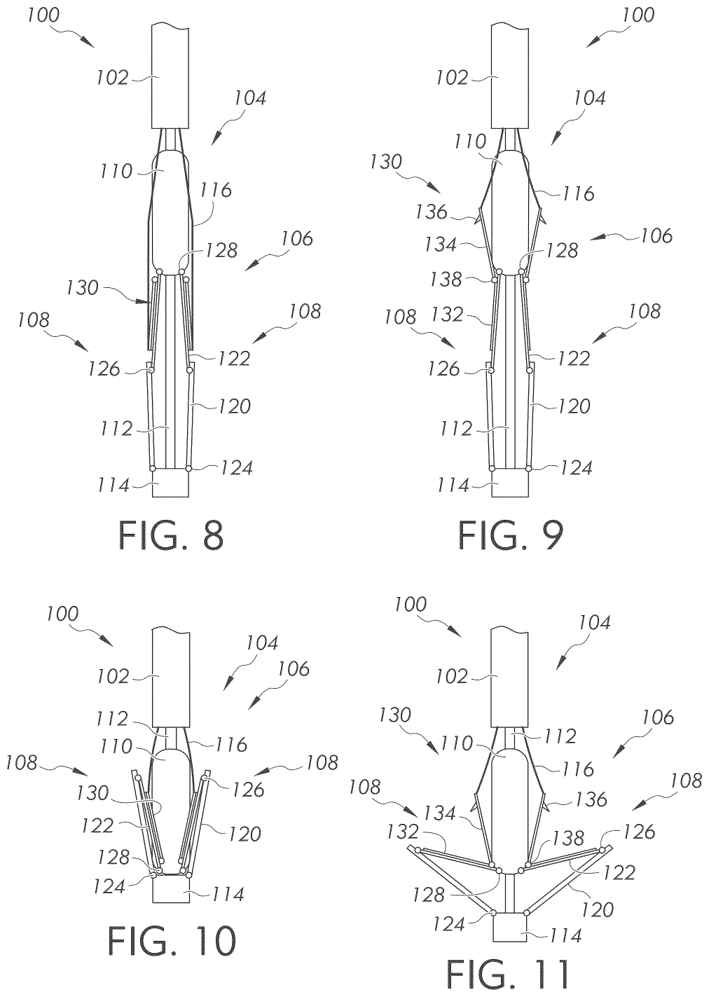

Referring now to FIGS. 8-15, a schematically illustrated implantable device or implant 100 (e.g., an implantable prosthetic device, a prosthetic spacer device, a valve repair device, etc.) is shown in various stages of deployment. The device or implant 100 and other similar devices/implants are described in more detail in PCT patent application publication Nos. WO2018/195215, WO2020/076898, and WO 2019/139904, which are incorporated herein by reference in their entirety for all purposes. The device 100 can include any other features for an implantable device or implant discussed in the present application or the applications cited above, and the device 100 can be positioned to engage valve tissue (e.g., leaflets 20, 22, 30, 32, 34) as part of any suitable valve repair system (e.g., any valve repair system disclosed in the present application or the applications cited above).

The device or implant 100 is deployed from a delivery system 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device or implant 100 includes a coaptation portion 104 and an anchor portion 106.

In some implementations, the coaptation portion 104 of the device or implant 100 includes a coaptation element 110 that is adapted to be implanted between leaflets of a native valve (e.g., a native mitral valve, native tricuspid valve, etc.) and is slidably attached to an actuation element 112 (e.g., actuation wire, actuation shaft, actuation tube, etc.). The anchor portion 106 includes one or more anchors 108 that are actuatable between open and closed conditions and can take a wide variety of forms, such as, for example, paddles, gripping elements, or the like. Actuation of the actuation element 112 opens and closes the anchor portion 106 of the device 100 to grasp the native valve leaflets during implantation. The actuation element 112 (as well as other actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and a coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and the actuation element 112 (e.g., actuation wire, actuation shaft, etc.). These can be configured to extend through a guide catheter/sheath (e.g., a transseptal sheath, etc.). In some implementations, the actuation element 112 extends through a delivery catheter and the coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively.

In some implementations, a collar or other attachment element removably attaches the coaptation element 110 to the delivery system 102, either directly or indirectly, so that the actuation element 112 slides through the collar or other attachment element and, in some implementations, through a coaptation element 110 during actuation to open and close the paddles 120, 122 of the anchor portion 106 and/or anchors 108.

In some implementations, the anchor portion 106 and/or anchors 108 can include attachment portions or gripping members. The illustrated gripping members can comprise clasps 130 that include a base or fixed arm 132, a moveable arm 134, optional friction-enhancing elements, other securing structures 136 (e.g., barbs, protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate the coaptation element 110. The joint portion 138 provides a spring force between the fixed and moveable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and moveable arms 132, 134. The fixed arms 132 are attached to the inner paddles 122 and remain stationary or substantially stationary relative to the inner paddles 122 when the moveable arms 134 are opened to open the clasps 130 and expose the optional barbs or other friction-enhancing elements 136.

In some implementations, the clasps 130 are opened by applying tension to actuation lines 116 attached to the moveable arms 134, thereby causing the moveable arms 134 to articulate, flex, or pivot on the joint portions 138. The actuation lines 116 extend through the delivery system 102 (e.g., through a steerable catheter and/or an implant catheter). Other actuation mechanisms (e.g., wires, rods, etc.) are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. Optional barbs or other friction-enhancing elements 136 of the clasps 130 can grab, pinch, and/or pierce the native leaflets to further secure the native leaflets.

During implantation, the paddles 120, 122 can be opened and closed, for example, to grasp the native leaflets (e.g., native mitral valve leaflets, etc.) between the paddles 120, 122 and/or between the paddles 120, 122 and a coaptation element 110 (e.g., a spacer, plug, membrane, etc.). The clasps 130 can be used to grasp and/or further secure the native leaflets by engaging the leaflets with optional barbs or other friction-enhancing elements 136 and pinching the leaflets between the moveable and fixed arms 134, 132. The optional barbs or other friction-enhancing elements 136 (e.g., protrusions, ridges, grooves, textured surfaces, adhesive, etc.) of the clasps or barbed clasps 130 increase friction with the leaflets or can partially or completely puncture the leaflets. The actuation lines 116 can be actuated separately so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the device 100 is shown in an elongated or fully open condition for deployment from an implant delivery catheter of the delivery system 102. The device 100 is disposed at the end of the catheter of the delivery system 102 in the fully open position. In the elongated condition the cap 114 is spaced apart from the coaptation element 110 such that the paddles 120, 122 are fully extended. In some implementations, an angle formed between the interior of the outer and inner paddles 120, 122 is approximately 180 degrees. The clasps 130 can be kept in a closed condition during deployment through the delivery system. The actuation lines 116 can extend and attach to the moveable arms 134.

Referring now to FIG. 9, the device 100 is shown in an elongated condition, similar to FIG. 8, but with the clasps 130 in a fully open position, ranging from about 140 degrees to about 200 degrees, from about 170 degrees to about 190 degrees, or about 180 degrees between fixed and moveable portions 132, 134 of the clasps 130.

Referring now to FIG. 10, the device 100 is shown in a shortened or fully closed condition. To move the device 100 from the elongated condition to the shortened condition, the actuation element 112 is retracted to pull the cap 114 towards the coaptation element 110. The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the coaptation element 110 cause the paddles to move radially outward. During movement from the open position to the closed position, the outer paddles 120 maintain an acute angle with the actuation element 112. The outer paddles 120 can optionally be biased toward a closed position. The inner paddles 122 during the same motion move through a considerably larger angle as they are oriented away from the coaptation element 110 in the open condition and collapse along the sides of the coaptation element 110 in the closed condition.

Referring now to FIGS. 11-13, the device 100 is shown in a partially open, grasp-ready condition. To transition from the fully closed to the partially open condition, the actuation element (e.g., actuation wire, actuation shaft, etc.) is extended to push the cap 114 away from the coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. In the example illustrated by FIG. 15, the device 100 can have two actuation elements 111, 113 and two independent caps 115, 117 (or other attachment portions), such that one independent actuation element (e.g., wire, shaft, etc.) and cap (or other attachment portion) are used to control one paddle, and the other independent actuation element and cap (or other attachment portion) are used to control the other paddle.

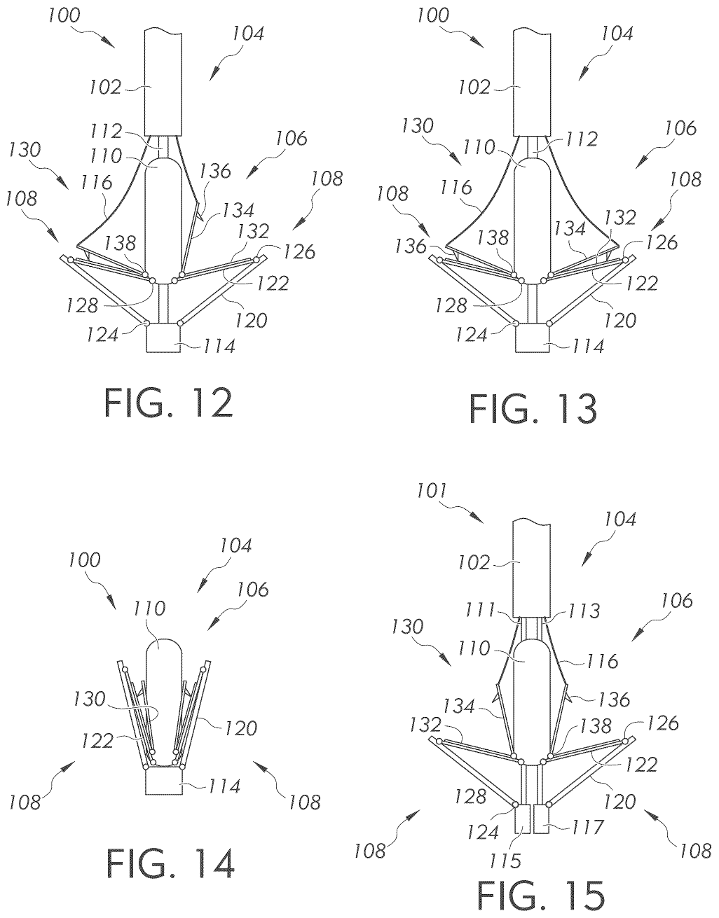

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system 102 and actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol—produced in a wire, sheet, tubing, or laser sintered powder—and are biased to hold the outer paddles 120 closed around the coaptation element 110 and the clasps 130 pinched around native leaflets. Similarly, the fixed and moveable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after implantation.

FIG. 15 illustrates an example where the paddles 120, 122 are independently controllable. The device 101 illustrated by FIG. 15 is similar to the device illustrated by FIG. 11, except the device 100 of FIG. 15 includes an actuation element that is configured as two independent actuation elements 111, 113 that are coupled to two independent caps 115, 117. To transition a first inner paddle 122 and a first outer paddle 120 from the fully closed to the partially open condition, the actuation element 111 is extended to push the cap 115 away from the coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the actuation element 113 is extended to push the cap 115 away from the spacer or coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single actuation element 112.

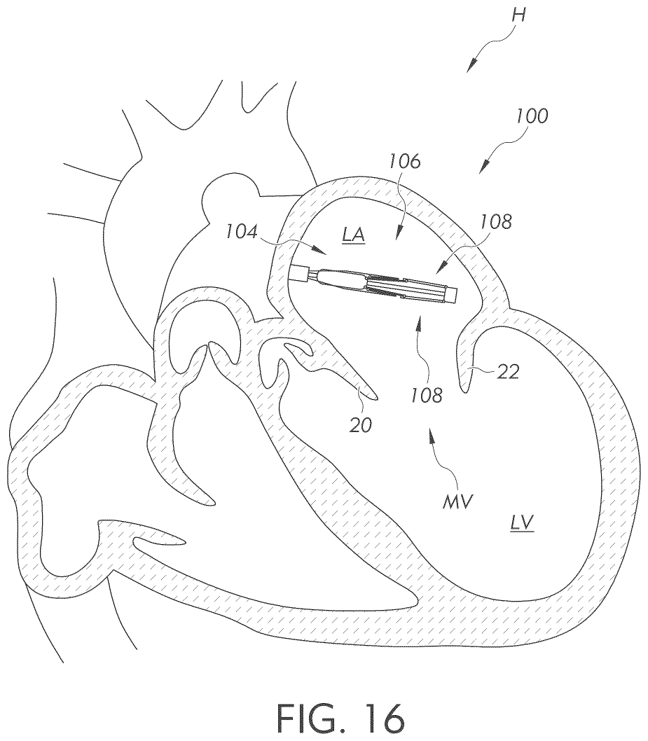

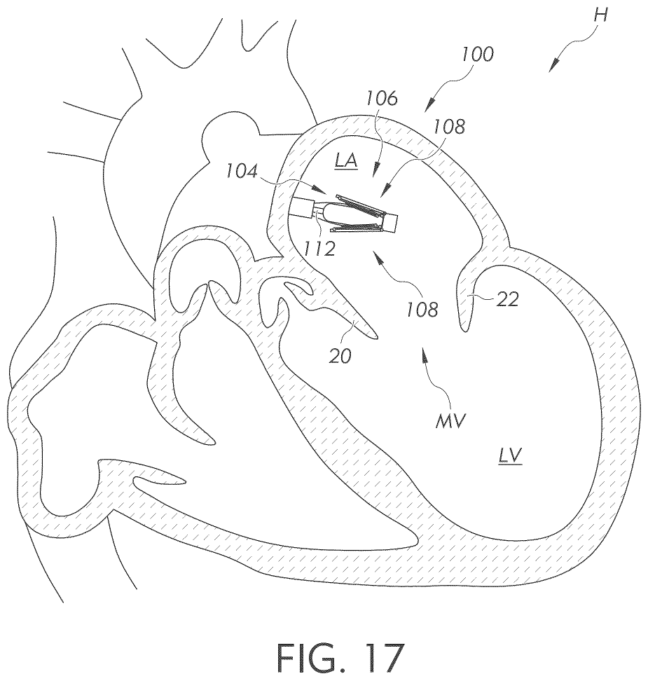

Referring now to FIGS. 16-21, the implantable device 100 of FIGS. 8-14 is shown being delivered and implanted within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The actuation element 112 is then retracted to move the implant/device into the fully closed condition shown in FIG. 17.

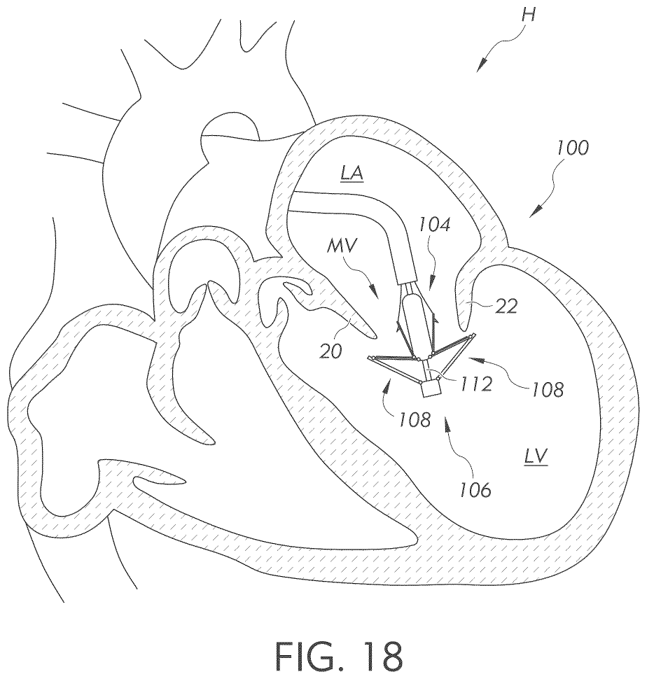

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The implant catheter connected to the implant/device can be advanced from inside the steerable catheter to position the implant as illustrated by FIG. 18.

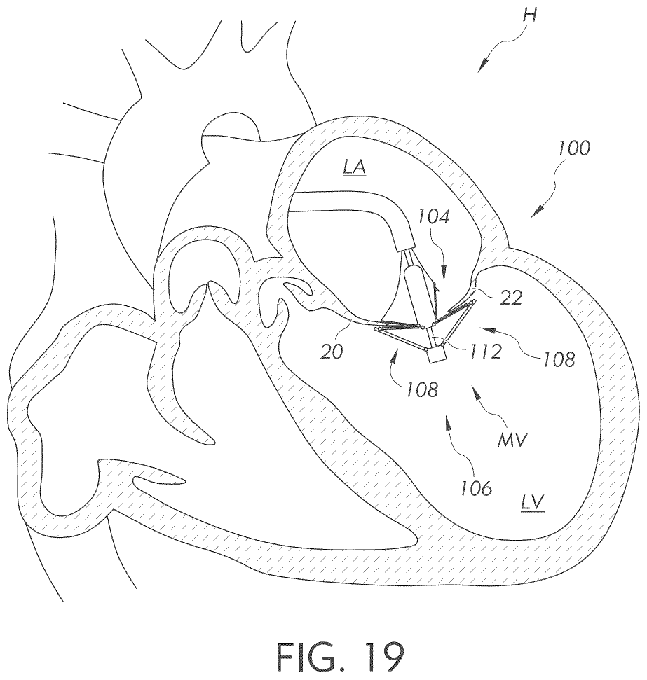

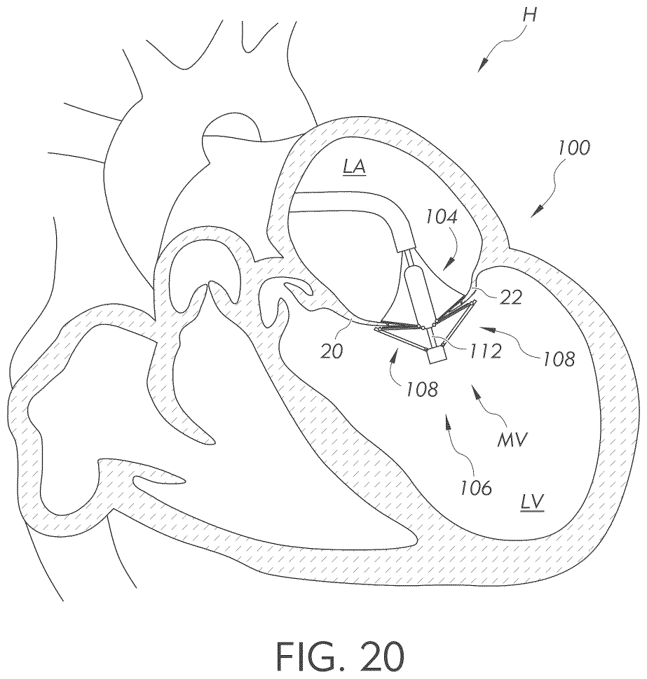

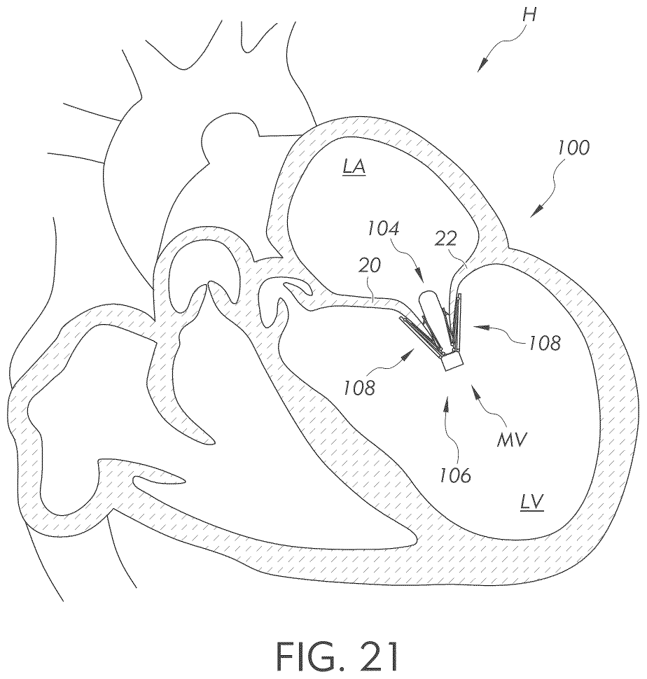

Referring now to FIG. 19, the implant catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), actuation element 112 and actuation lines 116 are then retracted and the device or implant 100 is fully closed and deployed in the native mitral valve MV.

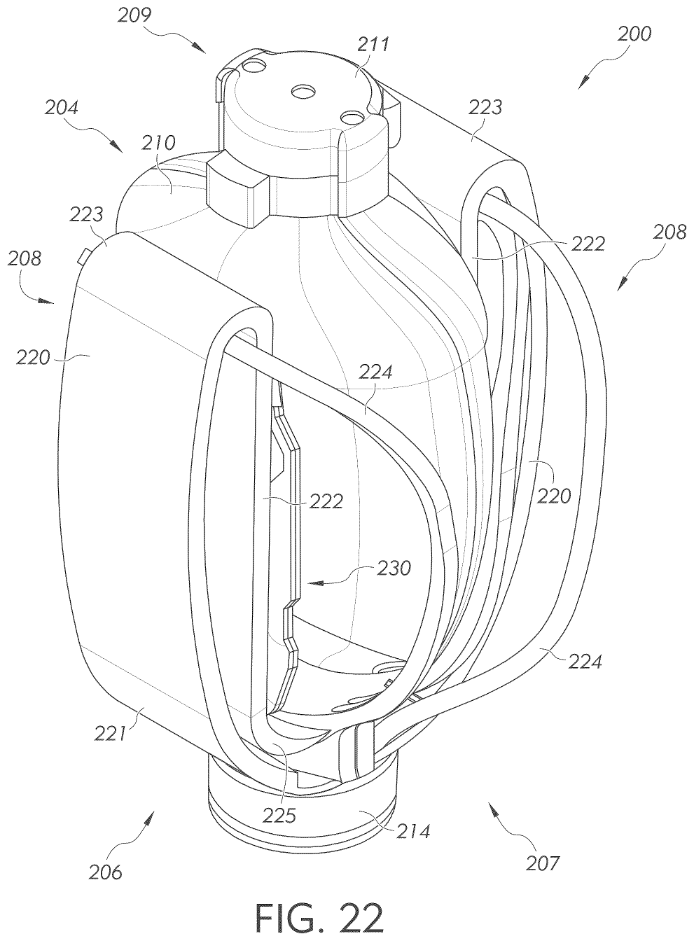

Any of the features disclosed by the present application can be used in a wide variety of different valve repair devices. FIGS. 22-24 illustrate examples of valve repair devices that can be modified to include any of the features disclosed by the present application. Any combination or sub-combination of the features disclosed by the present application can be combined with, substituted for, and/or added to any combination or sub-combination of the features of the valve repair devices illustrated by FIGS. 8-24.

Referring now to FIG. 22, an example of an implantable device or implant 200 is shown. The implantable device 200 is one of the many different configurations that the device 100 that is schematically illustrated in FIGS. 8-14 can take. The device 200 can include any other features for an implantable device or implant discussed in the present application, and the device 200 can be positioned to engage valve tissue 20, 22 as part of any suitable valve repair system (e.g., any valve repair system disclosed in the present application). The device/implant 200 can be a prosthetic spacer device, valve repair device, or another type of implant that attaches to leaflets of a native valve.

In some implementations, the implantable device or implant 200 includes a coaptation portion 204, a proximal or attachment portion 209, an anchor portion 206, and a distal portion 207. In some implementations, the coaptation portion 204 of the device optionally includes a coaptation element 210 (e.g., a spacer, coaption element, plug, membrane, sheet, gap filler, etc.) for implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 209 includes a first or proximal collar 211 (or other attachment element) for engaging with a capture mechanism (e.g., coupler, clamp, tether, etc.) of a delivery system. A delivery system for the device 200 can be the same as or similar to delivery system 102 described above and can comprise one or more of a catheter, a sheath, a guide catheter/sheath, a delivery catheter/sheath, a steerable catheter, an implant catheter, a tube, a channel, a pathway, combinations of these, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire-such as Nitinol—to provide shape-setting capability, or any other flexible material suitable for implantation in the human body.

An actuation element (e.g., actuation shaft, actuation rod, actuation tube, actuation wire, actuation line, etc.) can extend from a delivery system (not shown) to engage and enable actuation of the implantable device or implant 200. In some implementations, the actuation element extends through the proximal collar 211, and spacer or coaptation element 210 to engage a cap 214 of the distal portion 207. The actuation element can be configured to removably engage the cap 214 with a threaded connection, or the like, so that the actuation element can be disengaged and removed from the device 200 after implantation.

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above and has a tapered shape or cross-section when seen from a front view and a round shape or cross-section when seen from a side view. A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member (e.g., plate, bar, rod, panel, etc.) and/or a fixed portion of the clasps 230. The inner paddle 222, the outer paddle 220, and the coaptation element can all be interconnected as described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 can provide additional pinching force between the inner paddles 222 and the coaptation element 210 and assist in wrapping the leaflets around the sides of the coaptation element 210. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. For example, the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

The wide configuration of the paddle frames 224 provides increased surface area compared to the inner paddles 222 alone. The increased surface area can distribute the clamping force of the paddles 220 and paddle frames 224 against the native leaflets over a relatively larger surface of the native leaflets in order to further protect the native leaflet tissue.

Additional features of the device 200, modified versions of the device, delivery systems for the device, and methods for using the device and delivery system are disclosed by Patent Cooperation Treaty International Application No. PCT/US2018/028189 (International Publication No. WO 2018/195215). Any combination or sub-combination of the features disclosed by the present application can be combined with any combination or sub-combination of the features disclosed by Patent Cooperation Treaty International Application No. PCT/US2018/028189 (International Publication No. WO 2018/195215). Patent Cooperation Treaty International Application No. PCT/US2018/028189 (International Publication No. WO 2018/195215) is incorporated herein by reference in its entirety.

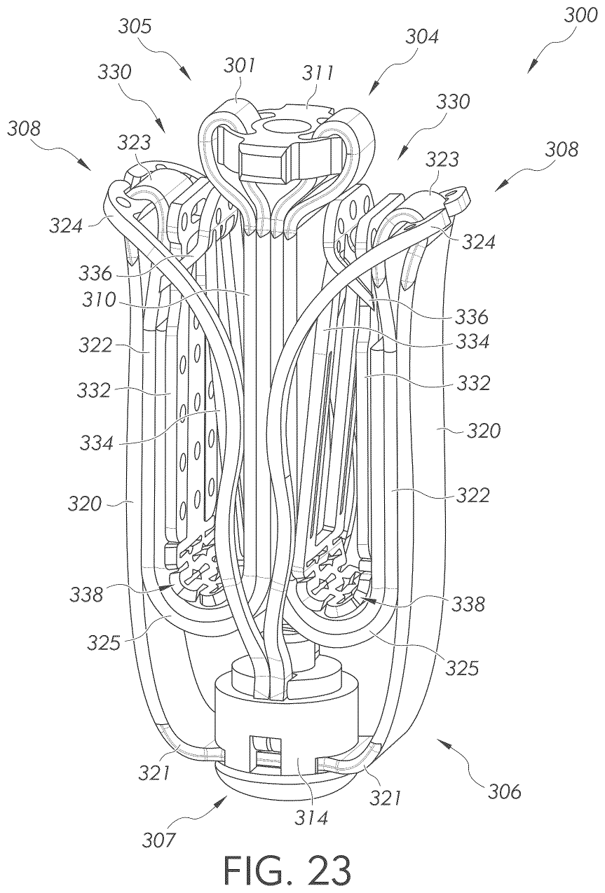

Referring now to FIG. 23, an example of an implantable device or implant 300 is shown. The implantable device 300 is one of the many different configurations that the device 100 that is schematically illustrated in FIGS. 8-14 can take. The device 300 can include any other features for an implantable device or implant discussed in the present application, and the device 300 can be positioned to engage valve tissue 20, 22 as part of any suitable valve repair system (e.g., any valve repair system disclosed in the present application).

The implantable device or implant 300 includes a proximal or attachment portion 305, an anchor portion 306, and a distal portion 307. In some implementations, the device/implant 300 includes a coaptation portion 304, and the coaptation portion 304 can optionally include a coaptation element 310 (e.g., spacer, plug, membrane, sheet, etc.) for implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members or paddle frames 324. The anchors can also include and/or be coupled to clasps 330. In some implementations, the attachment portion 305 includes a first or proximal collar 311 (or other attachment element) for engaging with a capture mechanism of a delivery system.

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In some implementations with a coaptation element 310, the coaptation element 310 and the anchors 308 can be coupled together in various ways. As shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from a continuous strip of fabric 301.

Like the anchors 208 of the implantable device or implant 200 described above, the anchors 308 can be configured to move between various configurations by axially moving the distal end of the device (e.g., cap 314, etc.) relative to the proximal end of the device (e.g., proximal collar 311 or other attachment element, etc.). This movement can be along a longitudinal axis extending between the distal end (e.g., cap 314, etc.) and the proximal end (e.g., collar 311 or other attachment element, etc.) of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the spacer or coaptation element 310. From the straight configuration, the anchors 308 can be moved to a fully folded configuration (e.g., FIG. 23), e.g., by moving the proximal end and distal end toward each other and/or toward a midpoint or center of the device.

In some implementations, the clasps comprise a moveable arm coupled to an anchor. In some implementations, the clasps 330 include a base or fixed arm 332, a moveable arm 334, optional barbs/friction-enhancing elements 336, and a joint portion 338. The fixed arms 332 are attached to the inner paddles 322, with the joint portion 338 disposed proximate the coaptation element 310. The joint portion 338 is spring-loaded so that the fixed and moveable arms 332, 334 are biased toward each other when the clasp 330 is in a closed condition.

The fixed arms 332 are attached to the inner paddles 322 through holes or slots with sutures. The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the moveable arms 334 are opened to open the clasps 330 and expose the optional barbs 336. The clasps 330 are opened by applying tension to actuation lines attached to the moveable arms 334, thereby causing the moveable arms 334 to articulate, pivot, and/or flex on the joint portions 338.

In short, the implantable device or implant 300 is similar in configuration and operation to the implantable device or implant 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 23 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 23, the strip of material 301 begins and ends in the location of the inner paddles 322.

As with the implantable device or implant 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which are wider than the coaptation element 310) at its widest is about 5 mm.

Additional features of the device 300, modified versions of the device, delivery systems for the device, and methods for using the device and delivery system are disclosed by Patent Cooperation Treaty International Application No. PCT/US2019/055320 (International Publication No. WO 2020/076898). Any combination or sub-combination of the features disclosed by the present application can be combined with any combination or sub-combination of the features disclosed by Patent Cooperation Treaty International Application No. PCT/US2019/055320 (International Publication No. WO 2020/076898). Patent Cooperation Treaty International Application No. PCT/US2019/055320 (International Publication No. WO 2020/076898) is incorporated herein by reference in its entirety.

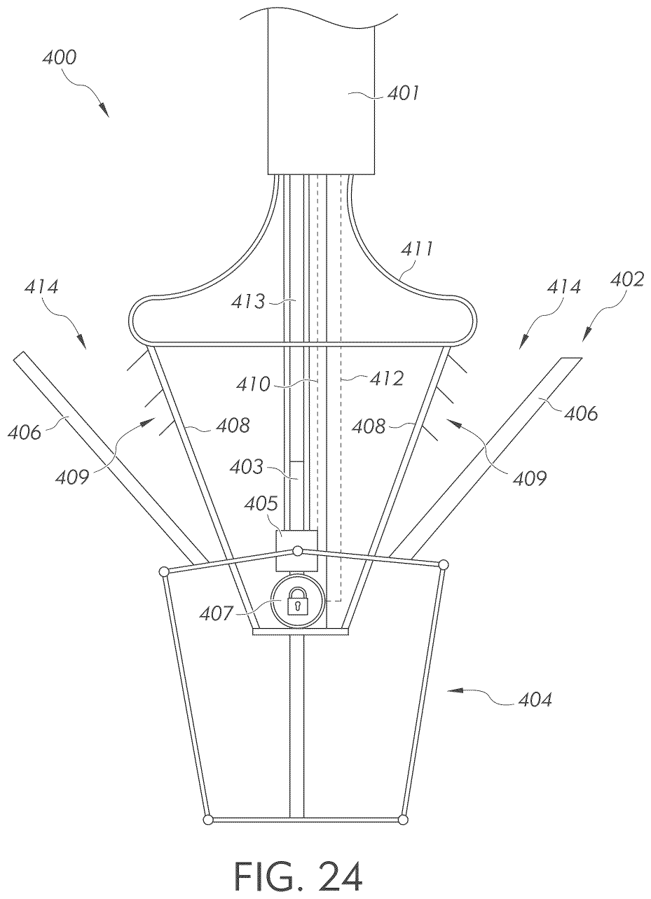

FIG. 24 illustrates another example of one of the many valve repair systems 400 for repairing a native valve of a patient that the concepts of the present application can be applied to. The valve repair system 400 includes a delivery device 401 and a valve repair device 402.

The valve repair device 402 includes a base assembly 404, a pair of paddles 406, and a pair of gripping members 408 (e.g., clasps). In one example, the paddles 406 can be integrally formed with the base assembly. For example, the paddles 406 can be formed as extensions of links of the base assembly. In the illustrated example, the base assembly 404 of the valve repair device 402 has a shaft 403, a coupler 405 configured to move along the shaft, and a lock 407 configured to lock the coupler in a stationary position on the shaft. The coupler 405 is mechanically connected to the paddles 406, such that movement of the coupler 405 along the shaft 403 causes the paddles to move between an open position and a closed position. In this way, the coupler 405 serves as a means for mechanically coupling the paddles 406 to the shaft 403 and, when moving along the shaft 403, for causing the paddles 406 to move between their open and closed positions.

In some implementations, the gripping members 408 are pivotally connected to the base assembly 404 (e.g., the gripping members 408 can be pivotally connected to the shaft 403, or any other suitable member of the base assembly), such that the gripping members can be moved to adjust the width of the opening 414 between the paddles 406 and the gripping members 408. The gripping member 408 can include an optional barbed portion 409 for attaching the gripping members to valve tissue when the valve repair device 402 is attached to the valve tissue. When the paddles 406 are in the closed position, the paddles engage the gripping members 408, such that, when valve tissue is attached to the barbed portion 409 of the gripping members, the paddles secure the valve repair device 402 to the valve tissue. In some implementations, the gripping members 408 are configured to engage the paddles 406 such that the barbed portion 409 engages the valve tissue member and the paddles 406 to secure the valve repair device 402 to the valve tissue member. For example, in certain situations, it can be advantageous to have the paddles 406 maintain an open position and have the gripping members 408 move outward toward the paddles 406 to engage valve tissue and the paddles 406.

While the example shown in FIG. 24 illustrates a pair of paddles 406 and a pair of gripping members 408, it should be understood that the valve repair device 402 can include any suitable number of paddles and gripping members.

In some implementations, the valve repair system 400 includes a placement shaft 413 that is removably attached to the shaft 403 of the base assembly 404 of the valve repair device 402. After the valve repair device 402 is secured to valve tissue, the placement shaft 413 is removed from the shaft 403 to remove the valve repair device 402 from the remainder of the valve repair system 400, such that the valve repair device 402 can remain attached to the valve tissue, and the delivery device 401 can be removed from a patient's body.

The valve repair system 400 can also include a paddle control mechanism 410 (e.g., rod, shaft, etc.), a gripper control mechanism 411 (e.g., line, wire, etc.), and a lock control mechanism 412 (e.g., line, wire, etc.). The paddle control mechanism 410 is mechanically attached to the coupler 405 to move the coupler along the shaft, which causes the paddles 406 to move between the open and closed positions. The paddle control mechanism 410 can take any suitable form, such as, for example, a shaft or rod. For example, the paddle control mechanism can comprise a hollow shaft, a catheter tube or a sleeve that fits over the placement shaft 413 and the shaft 403 and is connected to the coupler 405.

The gripper control mechanism 411 is configured to move the gripping members 408 such that the width of the opening 414 between the gripping members and the paddles 406 can be altered. The gripper control mechanism 411 can take any suitable form, such as, for example, a line, a suture or wire, a rod, a catheter, etc.

The lock control mechanism 412 is configured to lock and unlock the lock. The lock 407 locks the coupler 405 in a stationary position with respect to the shaft 403 and can take a wide variety of different forms and the type of lock control mechanism 412 can be dictated by the type of lock used. In examples in which the lock 407 includes a pivotable plate, the lock control mechanism 412 is configured to engage the pivotable plate to move the plate between the tilted and substantially non-tilted positions. The lock control mechanism 412 can be, for example, a rod, a suture, a wire, or any other member that is capable of moving a pivotable plate of the lock 407 between a tilted and substantially non-tilted position.

The valve repair device 402 is movable from an open position to a closed position. The base assembly 404 includes links that are moved by the coupler 405. The coupler 405 is movably attached to the shaft 403. In order to move the valve repair device from the open position to the closed position, the coupler 405 is moved along the shaft 403, which moves the links.

The gripper control mechanism 411 is moves the gripping members 408 to provide a wider or a narrower gap at the opening 414 between the gripping members and the paddles 406. In the illustrated example, the gripper control mechanism 411 includes a line, such as a suture, a wire, etc. that is connected to an opening in an end of the gripping members 408. When the line(s) is pulled, the gripping members 408 move inward, which causes the opening 414 between the gripping members and the paddles 406 to become wider.

In order to move the valve repair device 402 from the open position to the closed position, the lock 407 is moved to an unlocked condition by the lock control mechanism 412. Once the lock 407 is in the unlocked condition, the coupler 405 can be moved along the shaft 403 by the paddle control mechanism 410.

After the paddles 406 are moved to the closed position, the lock 407 is moved to the locked condition by the lock control mechanism 412 to maintain the valve repair device 402 in the closed position. After the valve repair device 402 is maintained in the locked condition by the lock 407, the valve repair device 402 is removed from the delivery device 401 by disconnecting the shaft 403 from the placement shaft 413. In addition, the valve repair device 402 is disengaged from the paddle control mechanism 410, the gripper control mechanism 411, and the lock control mechanism 412.

Additional features of the device 402, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904). Patent Cooperation Treaty International Application No. PCT/US2019/012707 (International Publication No. WO 2019139904) is incorporated herein by reference in its entirety.

Clasps or leaflet gripping devices disclosed herein can take a wide variety of different forms. Examples of clasps are disclosed by Patent Cooperation Treaty International Application No. PCT/US2018/028171 (International Publication No. WO 2018195201). Any combination or sub-combination of the features disclosed by the present application can be combined with any combination or sub-combination of the features disclosed by Patent Cooperation Treaty International Application No. PCT/US2018/028171 (International Publication No. WO 2018195201). Patent Cooperation Treaty International Application No. PCT/US2018/028171 (International Publication No. WO 2018195201) is incorporated herein by reference in its entirety.

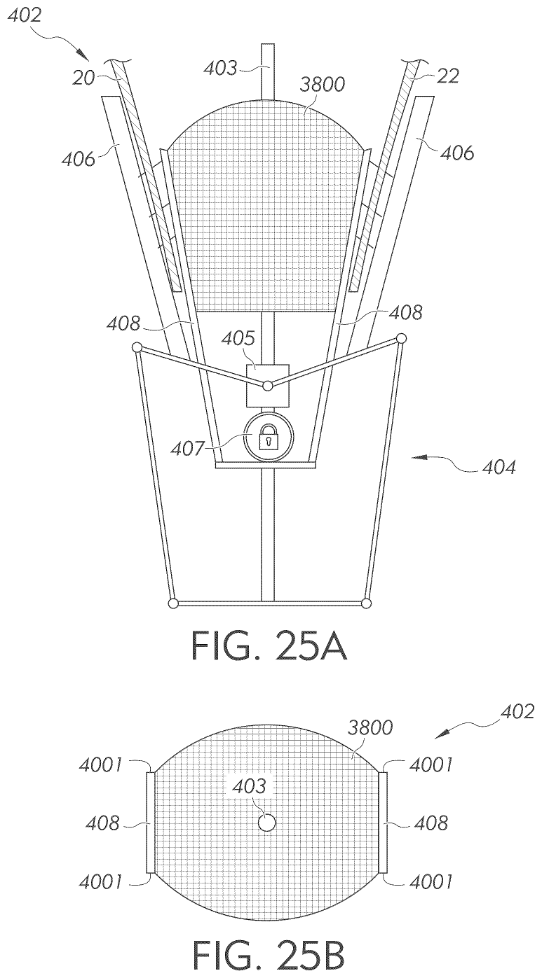

Referring to FIGS. 25A-25B, some implementations of a valve repair device 402 has a coaptation element 3800. The valve repair device 402 can have the same configuration as the valve repair device illustrated by FIG. 24 with the addition of the coaptation element. The coaptation element 3800 can take a wide variety of different forms. The coaptation element 3800 can be compressible and/or expandable. For example, the coaptation element can be compressed to fit inside one or more catheters of a delivery system, can expand when moved out of the one or more catheters, and/or can be compressed by the paddles 406 to adjust the size of the coaptation element.

In some implementations, for example the example illustrated by FIGS. 25A and 25B, the size of the coaptation element 3800 can be reduced by squeezing the coaptation element with the paddles 406 and can be increased by moving the paddles 406 away from one another. The coaptation element 3800 can extend past outer edges 4001 of the gripping members or clasps 408 as illustrated for providing additional surface area for closing the gap of a mitral valve.

The coaptation element 3800 can be coupled to the valve repair device 402 in a variety of different ways. For example, the coaptation element 3800 can be fixed to the shaft 403, can be slidably disposed around the shaft, can be connected to the coupler 405, can be connected to the lock 407, and/or can be connected to a central portion of the clasps or gripping members 408. In some implementations, the coupler 405 can take the form of the coaptation element 3800. That is, a single element can be used as the coupler 405 that causes the paddles 406 to move between the open and closed positions and the coaptation element 3800 that closes the gap between the leaflets 20, 22 when the valve repair device 402 is attached to the leaflets.

The coaptation element 3800 can be disposed around one or more of the shafts or other control elements of the valve repair system 400. For example, the coaptation element 3800 can be disposed around the shaft 403, the shaft 413, the paddle control mechanism 410, and/or the lock control mechanism 412.

The valve repair device 402 can include any other features for a valve repair device discussed in the present application, and the valve repair device 402 can be positioned to engage valve tissue as part of any suitable valve repair system (e.g., any valve repair system disclosed in the present application). Additional features of the device 402, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904).

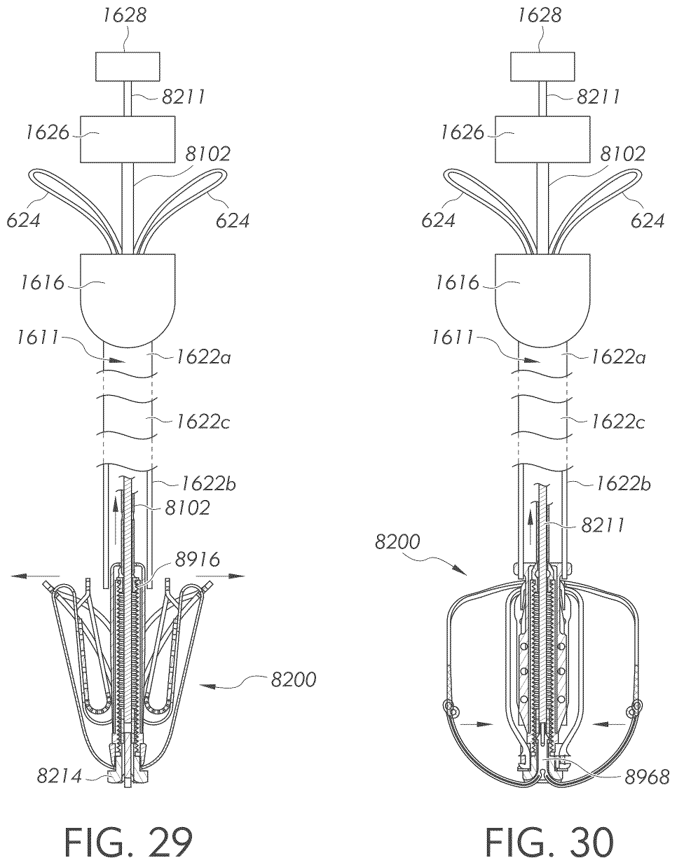

FIGS. 26-30 illustrate another example of one of the many valve repair systems for repairing a native valve of a patient that the concepts of the present application can be applied to. Referring to FIGS. 29 and 30, the valve repair system includes an implant catheter assembly 1611 and an implantable valve repair device 8200. Referring to FIGS. 26-28, the implantable device 8200 includes a proximal or attachment portion 8205, paddle frames 8224, and a distal portion 8207. The attachment portion 8205, the distal portion 8207, and the paddle frames 8224 can be configured in a variety of ways.

In the example illustrated in FIG. 26, the paddle frames 8224 can be symmetric along longitudinal axis YY. However, in some implementations, the paddle frames 8224 are not symmetric about the axis YY. Moreover, referring to FIG. 26, the paddle frames 8224 include outer frame portions 8256 and inner frame portions 8260.

In some implementations, the connector 8266 (e.g., shaped metal component, shaped plastic component, tether, wire, strut, line, cord, suture, etc.) attaches to the outer frame portions 8256 at outer ends of the connector 8266 and to a coupler 8972 at an inner end 8968 of the connector 8266 (see FIG. 28). Between the connector 8266 and the attachment portion 8205, the outer frame portions 8256 form a curved shape. In some implementations, e.g., as in the illustrated example, the shape of the outer frame portions 8256 resembles an apple shape in which the outer frame portions 8256 are wider toward the attachment portion 8205 and narrower toward the distal portion 8207. In some implementations, the outer frame portions 8256 can be otherwise shaped in a variety of ways, e.g., ovular, circular, triangular, rectangular, ovoid, etc.

The inner frame portions 8260 extend from the attachment portion 8205 toward the distal portion 8207. The inner frame portions 8260 then extend inward to form retaining portions 8272 that are attached to the actuation cap 8214. The retaining portions 8272 and the actuation cap 8214 can be configured to attach in any suitable manner.

In some implementations, the inner frame portions 8260 are rigid frame portions, while the outer frame portions 8256 are flexible frame portions. In some implementations, the proximal end of the outer frame portions 8256 connect to the proximal end of the inner frame portions 8260, as illustrated in FIG. 26.

In some implementations, the width adjustment element 8211 (e.g., width adjustment wire, width adjustment shaft, width adjustment tube, width adjustment line, width adjustment cord, width adjustment suture, width adjustment screw or bolt, etc.) is configured to move the outer frame portions 8256 from the expanded position to the narrowed position by pulling the inner end 8968 (FIG. 28) and portions of the connector 8266 into the actuation cap 8214. In some implementations, the actuation element 8102 is configured to move the inner frame portions 8260 to open and close the paddles in accordance with some implementations disclosed herein.

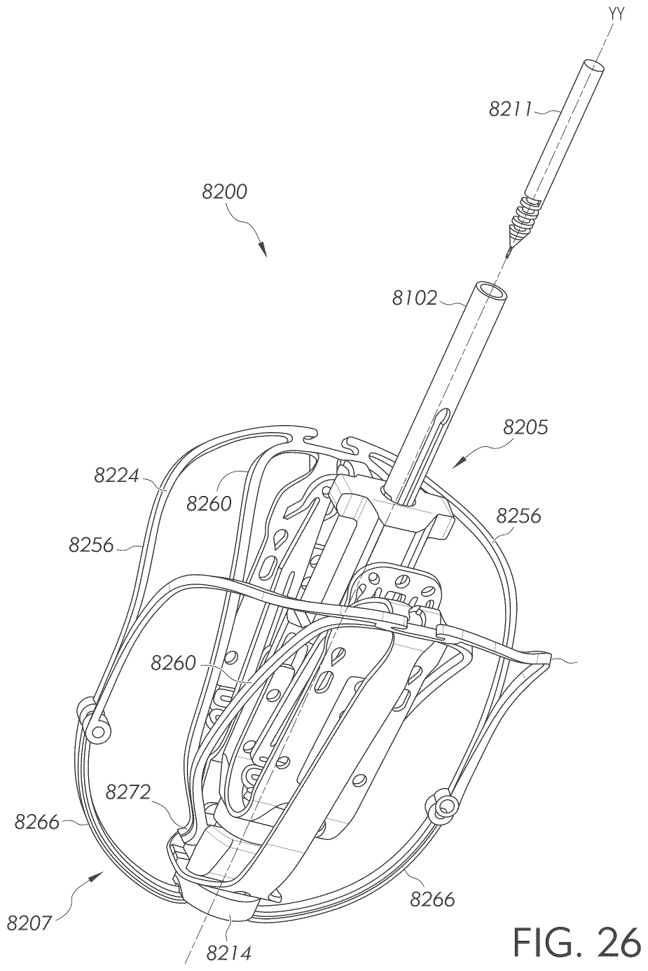

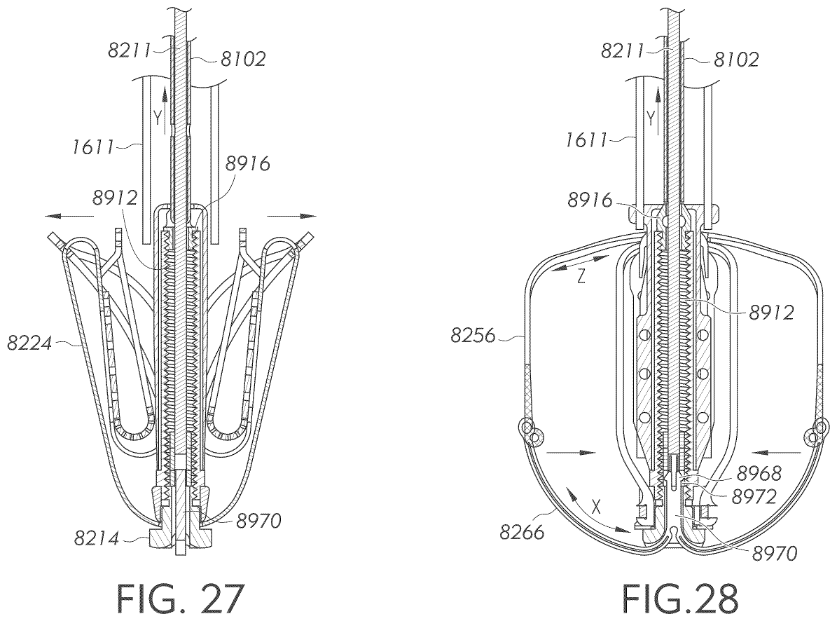

In some implementations, as shown in FIGS. 27 and 28, the connector 8266 has an inner end 8968 that engages with the width adjustment element 8211 such that a user can move the inner end 8968 inside the receiver 8912 (e.g., an internally threaded element, a column, a conduit, a hollow member, a notched receiving portion, a tube, a shaft, a sleeve, a post, a housing, a cylinder, tracks, etc.) to move the outer frame portions 8256 between a narrowed position and an expanded position. In the illustrated example, the inner end 8968 includes a post 8970 that attaches to the outer frame portions 8256 and a coupler 8972 that extends from the post 8970.

In some implementations, the coupler 8972 is configured to attach and detach from both the width adjustment element 8211 and the receiver 8912. The coupler 8972 can take a wide variety of different forms. For example, the coupler 8972 can include one or more of a threaded connection, features that mate with threads, detent connections, such as outwardly biased arms, walls, or other portions. In some implementations, when the coupler 8972 is attached to the width adjustment element 8211, the coupler is released from the receiver 8912. In some implementations, when the coupler 8972 is detached from the width adjustment element 8211, the coupler is secured to the receiver.

The inner end 8968 of the connector can be configured in a variety of ways. Any configuration that can suitably attach the outer frame portions 8256 to the coupler to allow the width adjustment element 8211 to move the outer frame portions 8256 between the narrowed position and the expanded position can be used. The coupler can be configured in a variety of ways as well and can be a separate component or be integral with another portion of the device, e.g., of the connector or inner end of the connector.

The width adjustment element 8211 allows a user to expand or contract the outer frame portions 8256 of the implantable device 8200. In the example illustrated in FIGS. 27 and 28, the width adjustment element 8211 includes an externally threaded end that is threaded into the coupler 8972.

In some implementations, the width adjustment element 8211 moves the coupler in the receiver 8912 to adjust the width of the outer frame portions 8256. In some implementations, when the width adjustment element 8211 is unscrewed from the coupler 8972, the coupler engages the inner surface of the receiver 8912 to set the width of the outer frame portions 8256.

In some implementations, the receiver 8912 can be integrally formed with a distal cap 8214. Moving the cap 8214 relative to a body of the attachment portion 8205 opens and closes the paddles. In the illustrated example, the receiver 8912 slides inside the body of the attachment portion. In some implementations, when the coupler 8972 is detached from the width adjustment element 8211, the width of the outer frame portions 8256 is fixed while the actuation element 8102 moves the receiver 8912 and cap 8214 relative to a body of the attachment portion 8205. Movement of the cap can open and close the device in the same manner as disclosed above.

In the illustrated example, a driver head 8916 is disposed at a proximal end of the actuation element 8102. The driver head 8916 releasably couples the actuation element 8102 to the receiver 8912. In the illustrated example, the width adjustment element 8211 extends through the actuation element 8102. The actuation element is axially advanced in the direction opposite to direction Y to move the distal cap 8214.

In some implementations, movement of the distal cap 8214 relative to the attachment portion 8205 is effective to open and close the paddles, as indicated by the arrows in FIG. 27. That is, movement of the distal cap 8214 in the direction Y closes the device and movement of the distal cap in the direction opposite to direction Y opens the device.

In some implementations, as illustrated in FIGS. 27 and 28, the width adjustment element 8211 extends through the actuation element 8102, the driver head 8916, and the receiver 8912 to engage the coupler 8972 attached to the inner end 8968. The movement of the outer frame portions 8256 to the narrowed position can allow the device or implant 8200 to maneuver more easily into position for implantation in the heart by reducing the contact and/or friction between the native structures of the heart—e.g., chordae—and the device 8200. The movement of the outer frame portions 8256 to the expanded position provides the anchor portion of the device or implant 8200 with a larger surface area to engage and capture leaflet(s) of a native heart valve.

Referring to FIGS. 29 and 30, an implementation of an implant catheter assembly 1611 in which clasp actuation lines 624 extend through a handle 1616, the actuation element 8102 is coupled to a paddle actuation control 1626, and the width adjustment element 8211 is coupled to a paddle width control 1628. A proximal end portion 1622a of the shaft or catheter of the implant catheter assembly 1611 can be coupled to the handle 1616, and a distal end portion 1622b of the shaft or catheter can be coupled to the implantable device 8200.

In some implementations, the actuation element 8102 can extend distally from the paddle actuation control 1626, through the handle 1616, through the delivery shaft or catheter of the implant catheter assembly 1611, and through the proximal end of the device 8200, where it couples with the driver head 8916. In some implementations, the actuation element 8102 can be axially movable relative to the outer shaft of the implant catheter assembly 1611 and the handle 1616 to open and close the device.

In some implementations, the width adjustment element 8211 can extend distally from the paddle width control 1628, through the paddle actuation control 1626 and through the actuation element 8102 (and, consequently, through the handle 1616, the outer shaft of the implant catheter assembly 1611, and through the device 8200), where it couples with the movable coupler 8972. In some implementations, the width adjustment element 8211 can be axially movable relative to the actuation element 8102, the outer shaft of the implant catheter assembly 1611, and the handle 1616.

In some implementations, the clasp actuation lines 624 can extend through and be axially movable relative to the handle 1616 and the outer shaft of the implant catheter assembly 1611. In some implementations, the clasp actuation lines 624 can also be axially movable relative to the actuation element 8102.

Referring to FIGS. 29 and 30, the width adjustment element 8211 can be releasably coupled to the coupler 8972 of the device 8200. In some implementations, advancing and retracting the width adjustment element 8211 with the paddle width control 1628 widens and narrows the paddles. In some implementations, advancing and retracting the actuation element 8102 with the paddle actuation control 1626 opens and closes the paddles of the device.

In the examples of FIGS. 29 and 30, the catheter or shaft of the implant catheter assembly 1611 is an elongate shaft extending axially between the proximal end portion 1622a, which is coupled to the handle 1616, and the distal end portion 1622b, which is coupled to the device 8200. In some implementations, the outer shaft of the implant catheter assembly 1611 can also include an intermediate portion 1622c disposed between the proximal and distal end portions 1622a, 1622b.

In some implementations, an implantable valve repair device can include a clip or connector (e.g., a frame clip, a frame connector, a paddle frame clip, a paddle frame connector, a strain reducer, etc.) that can attach various components of the implantable valve repair device to connect the components together and/or promote stabilization of various components, e.g., of the inner paddle, the outer paddle, the inner paddle frame, and/or the outer paddle frame. In some implementations, the clip/connector can add column strength and enhance the stiffness of the outer paddle frame to reduce buckling or rolling that can occur during narrowing of the implantable valve repair device.

In some implementations, the clip/connector can be configured as a paddle frame clip/connector that can couple the paddle frame portion to a portion of the paddle frame. In some implementations, the paddle frame clip/connector can secure at least one of the inner frame portion and the outer frame portion to at least one of the outer paddle and the inner paddle.

In some implantable valve repair devices, the frame portions are sutured to other components of the device, such as to other frame components, the paddle portions, etc. In some implementations, the clip/connector can reduce or eliminate the need for suturing the frame portions and other components of the device with sutures.

In some implementations, the paddle frame clip/connector is configured such that a stem portion couples with the inner paddle or the outer paddle, and a head portion couples with at least one of the outer frame portion and the inner frame portion.

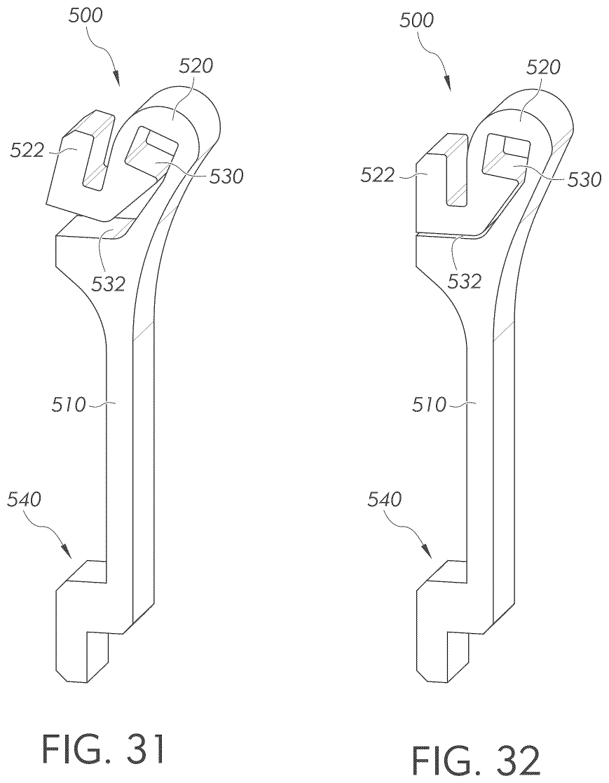

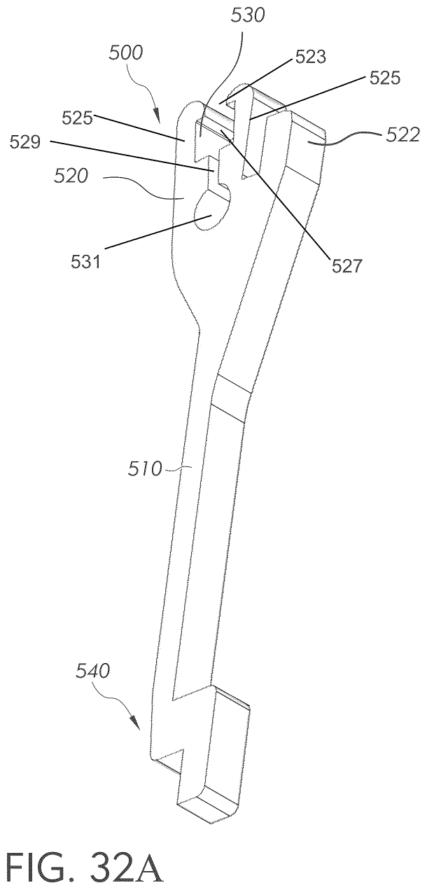

With reference to FIGS. 31 and 32, an example clip/connector 500 is illustrated. The clip/connector 500 can include a stem portion 510 and a head portion 520. The clip/connector can be made from a variety of materials, for example, steel, other metals, plastics, composites, etc. or shape-memory alloys such as Nitinol.

The clip(s)/connector(s) herein can be configured in a variety of different sizes and shapes. In some implementations, the stem portion is straight or comprises a straight section (e.g., a straight section extending a majority of the length of the stem). In some implementations, the stem portion is curved or comprises a curved section (e.g., a convex curve, a concave curve, an S-shaped curve, a sinusoidal curve, etc.) In some implementations, the stem portion comprises a zig zag section.

The head can have a variety of sizes and shapes as well. In some applications the head comprises a shape that is at least one of a circular, square, rectangular, triangular, ovoid, oval, semicircular, a combination of these, etc.

In some implementations, the head portion 520 of the paddle frame clip/connector 500 can include an aperture 530 extending through the paddle frame clip/connector 500. In some implementations, the aperture 530 can have a cross sectional shape that matches that of the inner frame portion or outer frame portion.

In some implementations, when the aperture 530 engages the inner frame portion or outer frame portion, the aperture 530 fits securely around the frame portion, reducing rotational movement of the inner frame portion or outer frame portion relative to the head portion 520. As illustrated in FIGS. 31-32, the aperture 530 can have a square shaped cross section, although the aperture can have a rectangular, circular, oval, triangular, or any other shaped cross section. In some implementations, the aperture can have an irregularly shaped cross section.

In some implementations, the head portion 520 can include one or more engaging members 522 (e.g., tongue, projection, extension, protrusion, etc.), which engages the inner paddle or the outer paddle. In some implementations, the stem portion 510 includes one or more engaging members 540 (e.g., tongue, projection, extension, protrusion, etc.), which can engage the inner paddle or the outer paddle. In some implementations, the engaging member 540 can be positioned at a distal end of the paddle frame clip/connector 500, opposite the head portion 520.

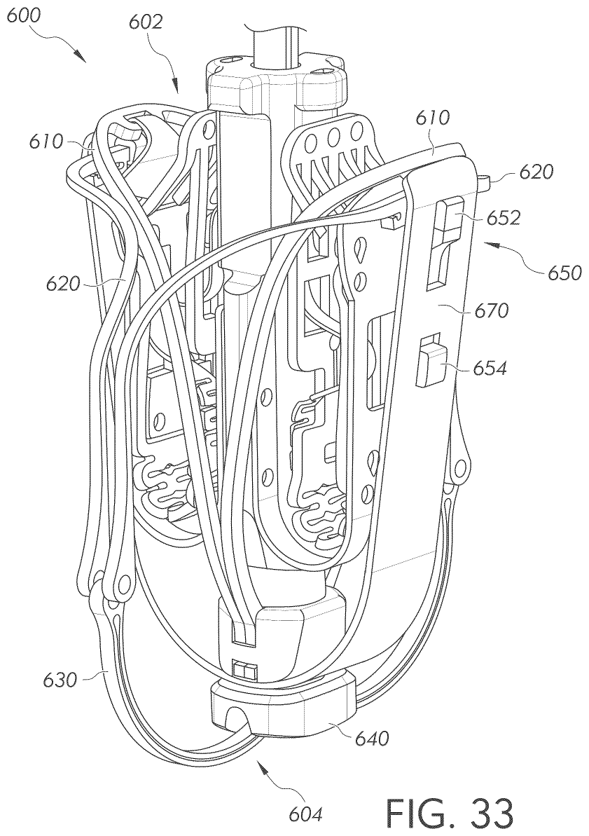

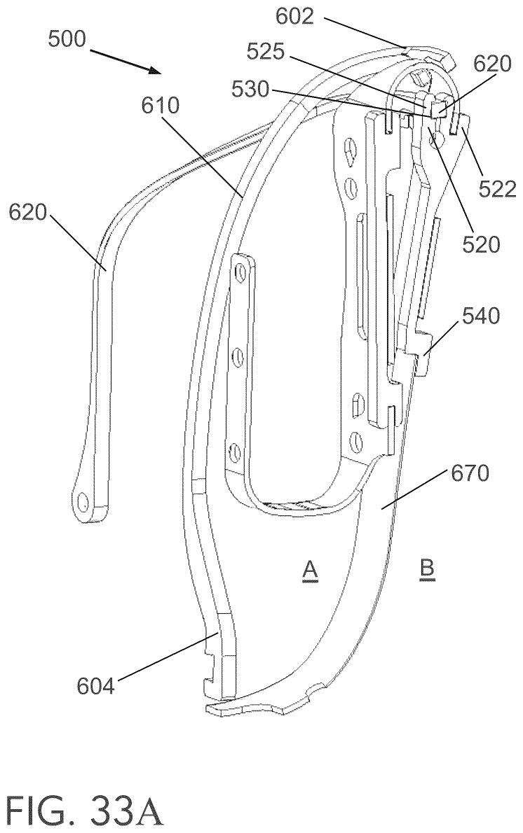

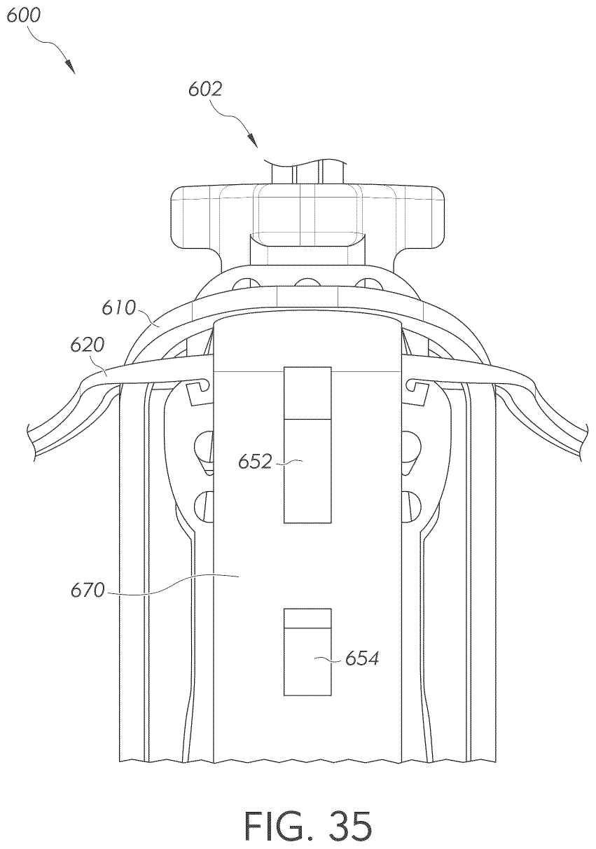

In some implementations, the engaging member 522 and the engaging member 540 can couple the paddle frame portion with the inner paddle or the outer paddle in a variety of ways. With reference to FIGS. 31 and 32, the engaging member 540 can comprise a step and an extension of the paddle frame clip/connector 500. In some implementations, the engaging member 540 can extend through and hook onto the outer paddle (see FIG. 33-35) or inner paddle (see FIG. 38). Likewise, in some implementations, the engaging member 522 can include a hook system which extends through and hooks onto the inner paddle or the outer paddle.

In some implementations, the paddle frame clip/connector 500 can have an open configuration (FIG. 31) and a closed configuration (FIG. 32). With reference to FIG. 31, in the open configuration, the head portion 520 is open such that the aperture 530 is available by way of a channel 532.

In some implementations, at least one of the inner frame portion or the outer frame portion can be moved through the channel 532 and be secured into the aperture 530. In the closed configuration (FIG. 32), the channel 532 is closed, securing the inner frame portion and/or the outer frame portion in the aperture 530.

In some implementations, the channel 532 is secured in the closed position when the clip/connector is installed in the inner paddle or the outer paddle. The inner or outer paddle can be securely connected to or coupled with the inner and/or outer paddle frame by the clip/connector.

In some implementations, the paddle frame clip/connector can be coupled to a paddle formed from sheet metals such as steel or shape-memory alloy, or from continuous strips of a braided or woven material, such as braided or woven nitinol wire (see, for example, FIG. 23).

Referring to FIG. 32A, in some implementations a clip/connector 500 includes an opening 523 through an end of a head 520. In some implementations, the clip/connector 500 illustrated by FIG. 32A can include a stem portion 510 and a head portion 520. The clip/connector 500 can be made from a variety of materials, for example, steel, other metals, plastics, composites, etc. or shape-memory alloys such as Nitinol.

In some implementations, the head portion 520 of the paddle frame clip/connector 500 can include an aperture 530 extending through the paddle frame clip/connector 500. In some implementations, the aperture 530 can have a cross sectional shape that matches that of the inner frame portion and/or outer frame portion.

In some implementations, when the aperture 530 engages the inner frame portion or outer frame portion, the aperture 530 fits securely around the frame portion, reducing rotational movement of the inner frame portion or outer frame portion relative to the head portion 520. As illustrated in FIG. 32A, the aperture 530 can have a square shaped cross section, although the aperture can have a rectangular, circular, oval, triangular, or any other shaped cross section. In some implementations, the aperture can have an irregularly shaped cross section.

In some implementations, the head portion 520 can include one or more engaging members 522, which engages the inner paddle or the outer paddle. In some implementations, the stem portion 510 includes one or more engaging members 540, which can engage the inner paddle or the outer paddle. The engaging member 540 can be positioned at a distal end of the paddle frame clip/connector 500, opposite the head portion 520.

The engaging member 522 and the engaging member 540 can couple the paddle frame portion with the inner paddle or the outer paddle in a variety of ways. With reference to FIG. 32A, the engaging member 540 can comprise a step and an extension of the paddle frame clip/connector 500. In some examples, the engaging member 540 can extend through and hook onto the outer paddle (see FIG. 32A). Likewise, in some implementations, the engaging member 522 can include a hook system which extends through and hooks onto the inner paddle.

In some implementations, the opening 523 of the head 520 is between two prongs 525. In some implementations, the two prongs 525 can be flexed to an open configuration and snap back or otherwise return to the closed configuration (FIG. 32A).

In some implementations, the head 520 can include an optional relief slot 529 and/or a relief hole 531 that allows or enhances the flexing of the prongs 525. In some implementations, at least one of the inner frame portion or the outer frame portion can flex the two prongs 525 away from one another and move through the opening 523 and into an aperture 530. In some implementations, the two prongs snap 525 back to the closed configuration to secure the inner and/or outer frame in the aperture 530.

In some implementations, in the closed configuration (FIG. 32A), flat, inner surfaces 527 of the prongs 525 engage the inner frame portion and/or the outer frame portion to prevent or inhibit the inner frame portion and/or the outer frame portion from moving back through the opening. In some implementations, one or more of the prongs 525 are secured in the closed position when the clip/connector is installed in the inner paddle or the outer paddle. As such, the inner or outer paddle can be securely assembled with the inner and/or outer paddle frame by the clip/connector.

In some implementations, the paddle frame clip/connector 500 of FIG. 32A can be coupled to a paddle formed from sheet metals such as steel or shape-memory alloy, or from continuous strips of a braided or woven material, such as braided or woven nitinol wire (see, for example, FIG. 23).

With reference to FIGS. 33-35, an example of an implantable valve repair device 600 is illustrated. In some implementations, the implantable valve repair device 600 can include any other features for an implantable valve repair device discussed in the present application, and the implantable valve repair device 600 can be positioned to engage valve tissue as part of any suitable valve repair system (e.g., any valve repair system disclosed in the present application).

In some implementations, the implantable valve repair device 600 comprises inner frame portions 610 and outer frame portions 620. In some implementations, between the connection portions 630 and the proximal portion 602, the outer frame portions 620 form a curved shape.

In some implementations, the inner frame portions 610 extends from the proximal portion 602 toward the distal portion 604. In some implementations, the inner frame portions 610 then extend inward to attach to the actuation cap 640. In some implementations, the inner frame portions 610 are rigid frame portions, while the outer frame portions 620 are flexible frame portions.

In some implementations, the outer frame portion 620 is connected to the outer paddle 670 by a paddle frame clip/connector 650. Specifically, an engaging member 652 (e.g., tongue, projection, extension, protrusion, etc.) and an engaging member 654 (e.g., tongue, projection, extension, protrusion, etc.) of the paddle frame clip/connector 650 are disposed through the outer paddle 670 and hook onto the outer paddle 670. The paddle frame clip/connector can have the same or similar configuration as the paddle frame clip/connector of FIGS. 31 and 32.

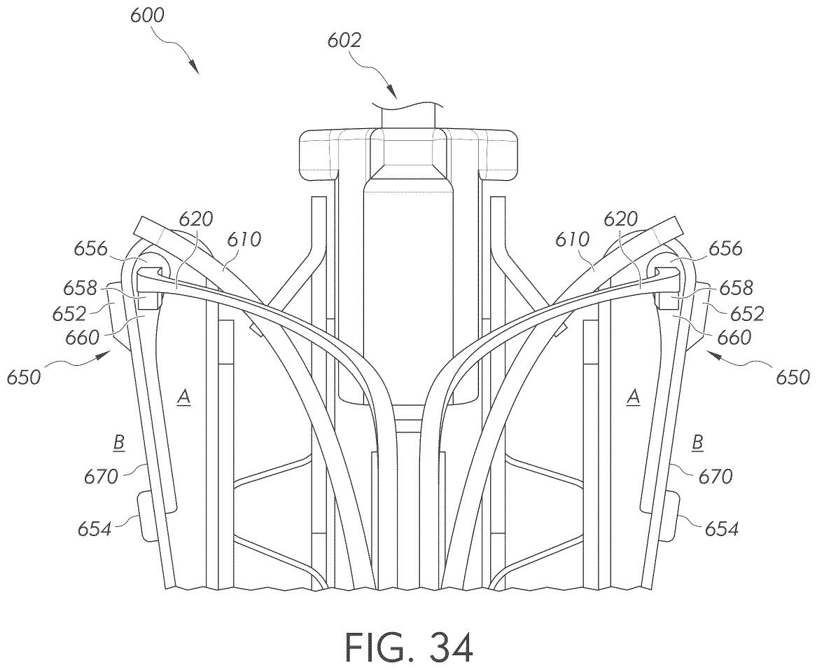

With reference to FIG. 34, in some implementations, the paddle frame clip/connector 650 is disposed from a first side A to a second side B of the outer paddle 670, with the engaging members 652, 654 hooking onto the outer paddle 670 at the second side B of the outer paddle 670.

In the example illustrated by FIG. 33, the head portion 656 of the paddle frame clip/connector 650 is coupled with the outer frame portion 620. The paddle frame clip/connector 650 is illustrated in the closed configuration, with the channel 660 being closed and the aperture 658 being secured around the outer frame portion 620. The channel 660 is closed by being installed on the outer paddle portion in some implementations.

With reference to FIGS. 33 and 33A, the implantable valve repair device 600 of FIG. 33 can have the clip/connector 500 of FIG. 32A. FIG. 33A illustrates the clip/connector 500 of FIG. 33A assembled with some of the components of the valve repair device 600 of FIG. 33. However, a valve repair device with the clip/connector 500 of FIG. 32A can include any other features for an implantable valve repair device discussed in the present application, and the implantable valve repair device can be positioned to engage valve tissue as part of any suitable valve repair system (e.g., any valve repair system disclosed in the present application).

In some implementations, the implantable valve repair device comprises inner frame portions 610 and outer frame portions 620. In some implementations, the outer frame portions 620 have a curved shape. In some implementations, the inner frame portions 610 extends from the proximal portion 602 toward the distal portion 604. In some implementations, the inner frame portions 610 then extend inward to attach to the actuation cap 640 (See FIG. 33). In some implementations, the inner frame portions 610 are rigid frame portions, while the outer frame portions 620 are flexible frame portions.

In some implementations, the outer frame portion 620 is connected to the outer paddle 670 by a paddle frame clip/connector 500. Specifically, an engaging member 522 and an engaging member 540 of the paddle frame clip/connector 500 are disposed through the outer paddle 670 and hook onto the outer paddle 670. The paddle frame clip/connector can have the same or similar configuration as the paddle frame clip/connector of FIG. 32A. With reference to FIG. 33A, the paddle frame clip/connector 500 is disposed from a first side A to a second side B of the outer paddle 670, with the engaging members 522, 540 hooking onto the outer paddle 670 at the second side B of the outer paddle 670.

In the example illustrated by FIG. 33A, the head portion 520 of the paddle frame clip/connector 500 is coupled with the outer frame portion 620. The paddle frame clip/connector 500 is illustrated in the closed configuration, with the prongs 525 being closed and the aperture 530 being secured around the outer frame portion 620. The prongs 525 are closed by being installed on the outer frame portion 620 in some implementations.

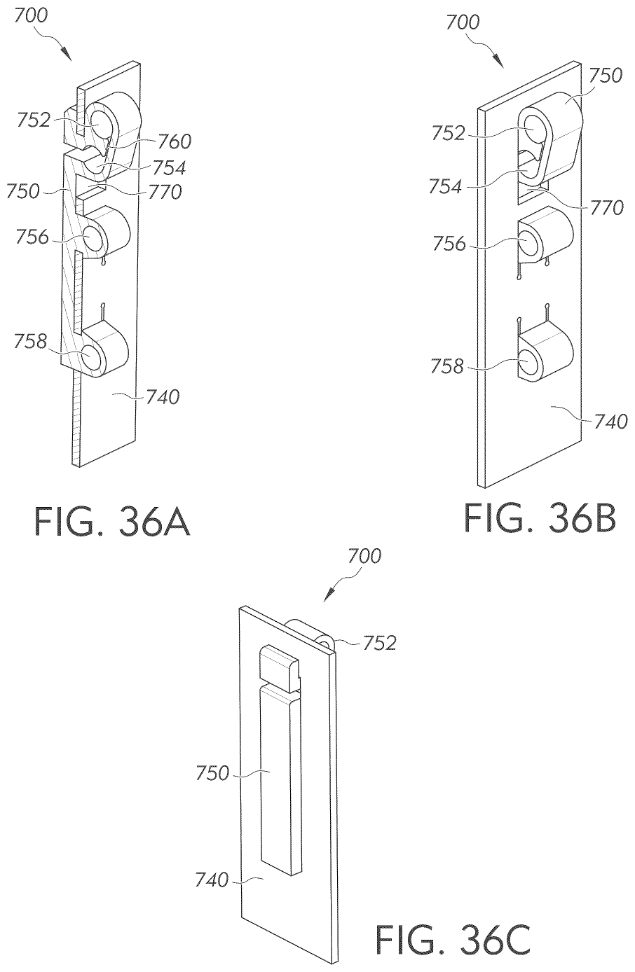

With reference to FIG. 36A-36C, another example of a paddle frame clip/connector 750 is illustrated through a cross section of a paddle 740. The paddle 740 can comprise the inner paddle or the outer paddle.

In some implementations, the paddle frame clip/connector 750 comprises a first aperture 752, a second aperture 754, a third aperture 756, and a fourth aperture 758. Any of the first aperture 752, second aperture 754, third aperture 756, or fourth aperture 758 can engage the inner frame portion or outer frame portion.

In some implementations, the first aperture 752 is coupled to the inner frame portion and the second aperture 754 is coupled to the outer frame portion, while in some implementations, the first aperture 752 is coupled to the outer frame portion and the second aperture 754 is coupled to the inner frame portion.

In some implementations, at least one of the third aperture 756 and fourth aperture 758 are coupled to other components of the implantable valve repair device 700 to assist in stabilizing the paddle frame clip/connector 750 to the paddle 740. In some implementations, the third aperture 756 and fourth aperture 758 can be coupled via a coupling device or a suture to promote further stabilization of the implantable valve repair device 600.

In some implementations, the apertures 756, 758 can be used as connection points for connecting a cover. For example, stitches that secure the cover to the paddles can pass through the cover material and the apertures 756, 758.

In some implementations, a channel 760 extends from the first aperture 752 to the second aperture 754. In use, the paddle frame clip/connector 750 can be coupled to the inner frame portion or outer frame portion first, and then disposed through the paddle 740. For example, one of the inner frame portion or outer frame portion can be disposed through the channel 760 and into the first aperture 752. Thereafter, the other of the inner frame portion or outer frame portion can be disposed through the channel 760 and into the second aperture 754. Thereafter, the paddle frame clip/connector 750 can be inserted into an opening 770 of the paddle 740 and arranged such that the engaging members engage to the corresponding openings in the paddle 740.

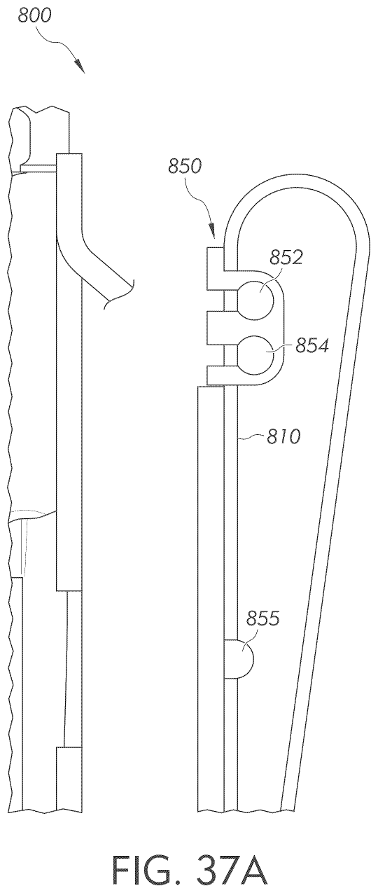

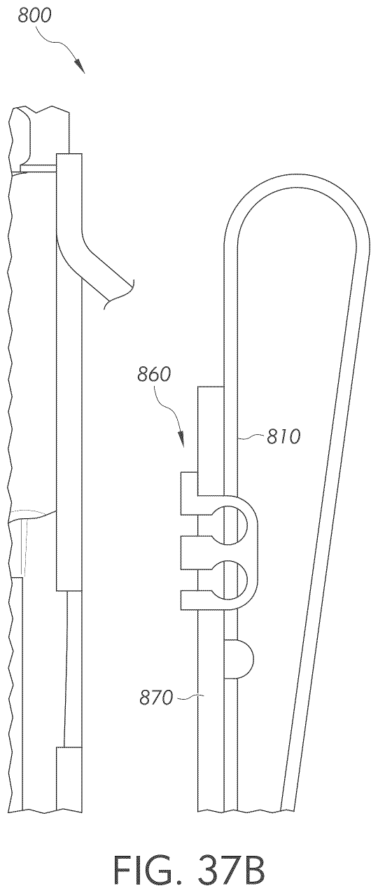

With reference to FIG. 37A, a paddle frame clip/connector 850 can be coupled to an inner paddle 810 of the implantable valve repair device 800. In some implementations, the paddle frame clip/connector 850 comprises a first aperture 852 and a second aperture 854. In some implementations, the first aperture 852 and second aperture 854 can engage either the inner frame portion or outer frame portion.

In some implementations, the first aperture 852 is coupled to the inner frame portion and the second aperture 854 is coupled to the outer frame portion, while in some implementations, the first aperture 852 is coupled to the outer frame portion and the second aperture 854 is coupled to the inner frame portion.

In some implementations the first aperture 852 and a second aperture 854 can be tangent to the surface of the inner paddle 810. Thus, the inner frame portion and outer frame portion can be positioned as close as possible or against the inner paddle 810.

In some implementations, the clip/connector 850 can extend along the inner paddle 810 to promote stiffening of the inner paddle 810, such that when pulling down on the inner paddle with the outer paddle, the inner paddle is reinforced and buckling of the inner paddle is inhibited. For example, the clip/connector can extend along the inner paddle 810 to a second attachment point 855 that extends through the inner paddle 810. With reference to FIG. 37B, in some implementations, the paddle frame clip/connector 860 can be coupled to and/or integral with the fixed arm of the clasp 870.

With reference to FIG. 38A-38B, an example implantable valve repair device 900 is illustrated with various components removed for viewing purposes. The outer frame portion 940 can include a first portion 942 and a second portion 944. In some implementations, the first portion 942 and the second portion 944 can pivot about one or more pins that couple the outer frame portion 940 to the inner frame portion 930.

In some implementations, the surface of the inner frame portion 930 can include one or more indents 932 to facilitate coupling of the frame portions with the paddle frame clip/connector 950 (see FIG. 38B) and/or to constrain movement of the paddle frame clip/connector 950.

With reference to FIG. 38B, a paddle frame clip/connector 950 can be coupled to a paddle (not shown) of the implantable valve repair device 900. In some implementations, the head portion 910 of the paddle frame clip/connector 950 comprises an aperture 952, which can engage at least one of the inner frame portion 930 or outer frame portion 940. As shown in FIG. 38B, the aperture 952 can be coupled to both the inner frame portion 930 and the outer frame portion 940. FIG. 38A illustrates the inner frame portion 930 and outer frame portion 940 without the paddle frame clip/connector 950 attached.

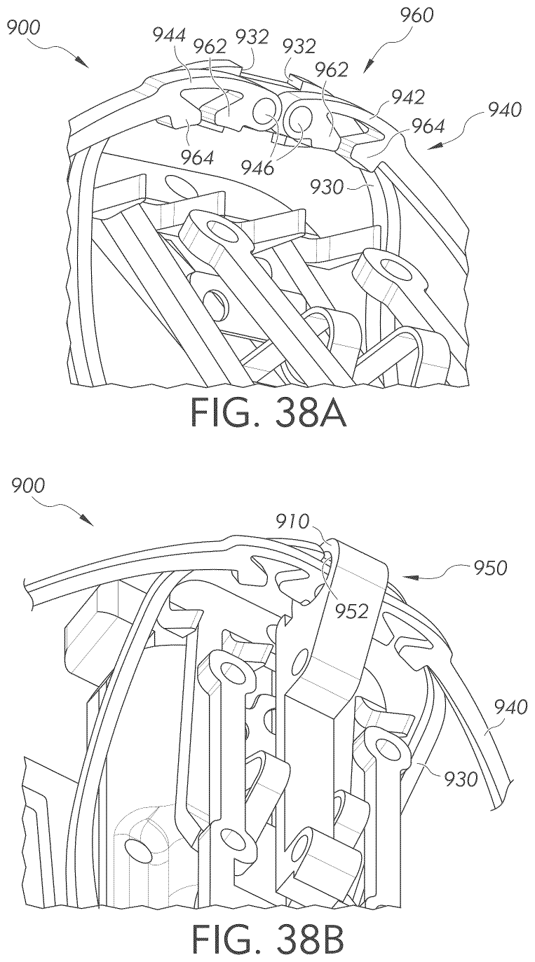

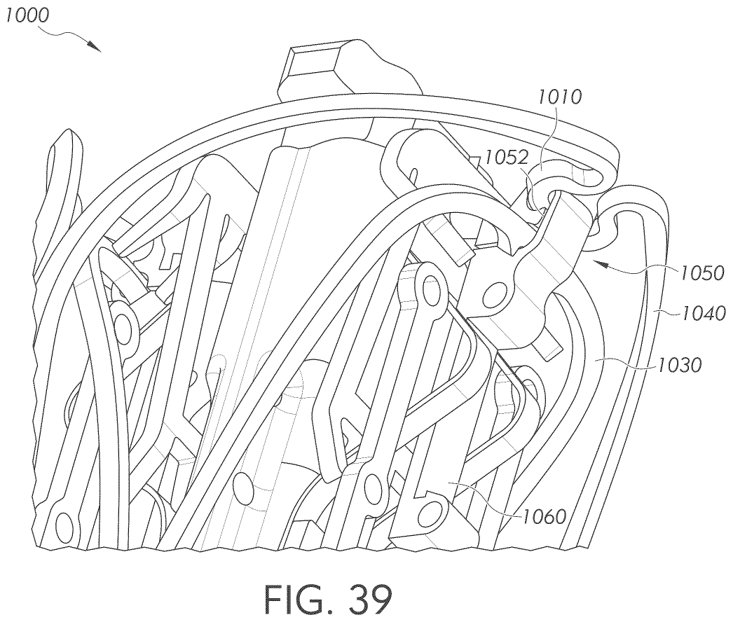

With reference to FIG. 39, an example implantable valve repair device 1000 is illustrated with various components removed for viewing purposes. A paddle frame clip/connector 1050 can be coupled to a paddle (not shown) of the implantable valve repair device 1000. For example, in some implementations, the stem portion 1060 of the clip/connector 1050 would extend along the inner paddle that is not shown so that the clip/connector is visible in FIG. 39. In some implementations, the head portion 1010 of the paddle frame clip/connector 1050 comprises one or more curves as well as an aperture 1052, which can engage both the inner frame portion 1030 and the outer frame portion 1040.

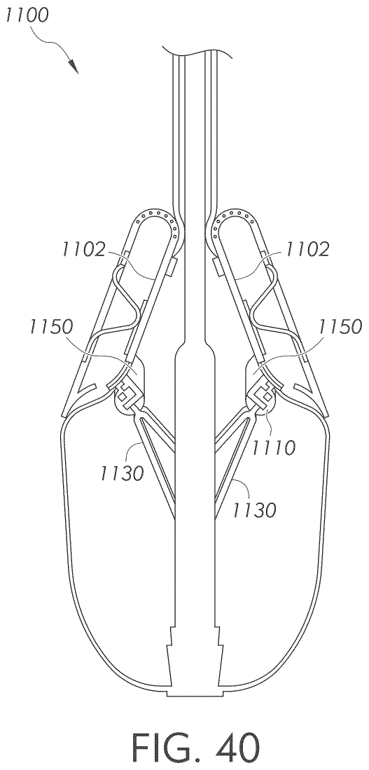

With reference to FIG. 40, an implantable valve repair device 1100 includes a paddle frame clip/connector 1150 coupled to the inner paddle and/or attached clasp 1102. In some implementations, the head portion 1110 of the paddle frame clip/connector 1150 engages a paddle frame 1130, which can include one or both of the inner frame portion and the outer frame portion. In FIG. 40, components of the device (e.g., the coaptation element, cap, coupler, actuation element, paddle frames, etc.) are illustrated generically to show the positions of the clip/connector 1150 when the device is in an elongated configuration and the clasps are closed.

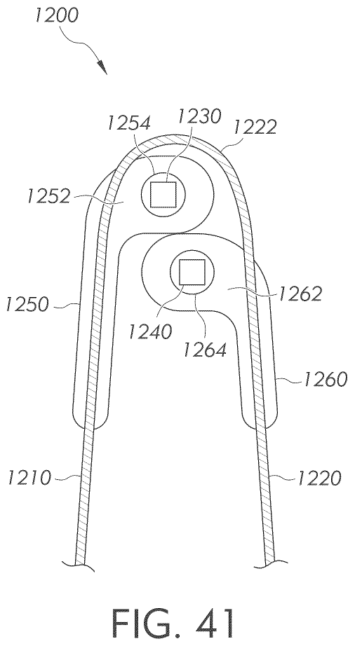

With reference to FIG. 41, a portion of an implantable valve repair device 1200 is illustrated. In some implementations, the implantable valve repair device 1200 can include a first paddle frame clip/connector 1250 coupled with the inner paddle 1210, as well as a second paddle frame clip/connector 1260 coupled with the outer paddle 1220. In some implementations, the first paddle frame clip/connector 1250 can comprise a head portion 1252, which includes an aperture 1254 through which one of the inner frame portion 1230 or the outer frame portion 1240 can be positioned. In some implementations, the second paddle frame clip/connector 1260 can comprise a head portion 1262, which includes an aperture 1264 through which one of the inner frame portion 1230 or the outer frame portion 1240 can be positioned.

With reference to FIG. 41, the inner frame portion 1230 can be disposed through the aperture 1254 of the first paddle frame clip/connector 1250, and the outer frame portion 1240 can be disposed through the aperture 1264 of the second paddle frame clip/connector 1260. In some implementations, when the implantable valve repair device 1200 is opened, the first paddle frame clip/connector 1250 and the second paddle frame clip/connector 1260 can make contact with one another, reducing rolling and distal translation of the outer paddle 1220 during opening and closing of the device. That is, the engagement between the clips or connectors 1250, 1260 can prevent or inhibit a transition portion 1222 between the inner and outer paddles 1210, 1220 from flexing downward without a corresponding movement of the inner or outer paddle frame portions 1230, 1240.

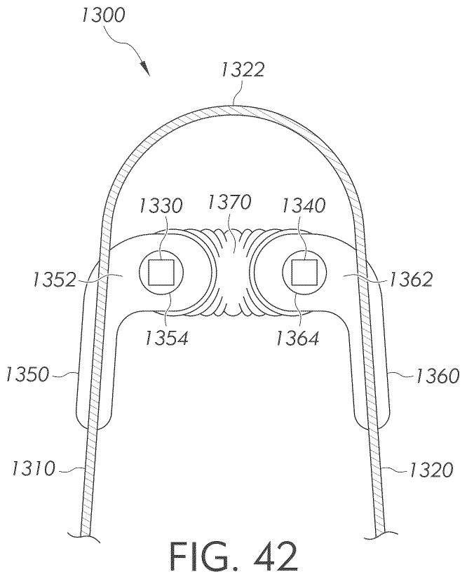

With reference to FIG. 42, a portion of an implantable valve repair device 1300 is illustrated. In some implementations, the implantable valve repair device 1300 can include a first paddle frame clip/connector 1350 coupled with the inner paddle 1310, as well as a second paddle frame clip/connector 1360 coupled with the outer paddle 1320. In some implementations, the first paddle frame clip/connector 1350 can comprise a head portion 1352, which includes an aperture 1354 through which one of the inner frame portion 1330 or the outer frame portion 1340 can be positioned through. In some implementations, the second paddle frame clip/connector 1360 can comprise a head portion 1362, which includes an aperture 1364 through which one of the inner frame portion 1330 or the outer frame portion 1340 can be positioned through.

With reference to FIG. 42, in some implementations, the inner frame portion 1330 is disposed through the aperture 1354 of the first paddle frame clip/connector 1350, and the outer frame portion 1340 is disposed through the aperture 1364 of the second paddle frame clip/connector 1360.

In some implementations, the implantable valve repair device 1300 also comprises a connecting member 1370 (e.g., flexible material, spring, strut, etc.) coupling the first paddle frame clip/connector 1350 and the second paddle frame clip/connector 1360. The connecting member 1370 can take a variety of shapes and be made of a variety of materials. The connecting member 1370 can be integrally formed with one or both of the clips/connectors 1350, 1360 or the connecting member 1370 can be a separate member disposed between the clips/connectors 1350, 1360. In some implementations, the connecting member 1370 comprises a material that is able to be compressed and/or stretched in a controlled manner. For example, the connecting member 1370 can be a spring.

In some implementations, when the implantable valve repair device 1300 is opened, the first paddle frame clip/connector 1250 and the second paddle frame clip/connector 1260 make contact with the connecting member 1370. In some implementations, the connecting member compresses and/or bends to control the relative movement between the clips/connectors 1350, 1360.

In some implementations, the controlled movement of the clips/connectors 1350, 1360 reduces rolling and distal translation of the outer paddle 1320 during opening and closing of the device. That is, the engagement of the connecting member 1370 with the clips/connectors 1350, 1360 can prevent or inhibit a transition portion 1322 between the inner and outer paddles 1310, 1320 from flexing downward without a corresponding movement of the inner or outer paddle frame portions 1330, 1340.

In some implementations, the outer frame portion can include a flex limiting portion. The flex limiting portion can comprise one or more projections, arms, cutouts, stops, gaps, holes, channels, apertures, and/or breaks etc. The flex limiting portion can comprise any structure that allows a portion of the outer paddle frame to flex a predetermined amount and then stop further flexing of the portion of the outer paddle frame when the predetermined amount of flexure is reached. The flex limiting portion can be integrally formed with the outer frame portion and/or can be a separate component that is connected to the outer frame portion. The flex limiting portion can be disposed at one or more portions of the outer paddle frame portion. For example, the flex limiting portion can be at the proximal end of the outer frame portion.

In some implementations, upon a bending of the outer frame portion, the flex limiting portion can reconfigure or bend until such time that the flex limiting portion engages to stop any further bending of the portion. In some implementations, once the flex limiting portion engages and stops flexing, other portions of the paddle frame can bend or flex. As such, the flex limiting portions can be used to control the flexing or bending of the outer frame portion. In some implementations, as the implantable valve repair device is narrowed, the flex limiting portion limits bending at the proximal end of the outer frame portion. In some implementations, the flex limiting portion allows other portions of the paddle frame to bend after the stop of the flex limiting portion has been reached. This staged or controlled bending of the paddle frame can be used to maintain or substantially maintain the depth at which the paddle frames engage the leaflets from the widest to the narrowest configurations of the paddle frames.

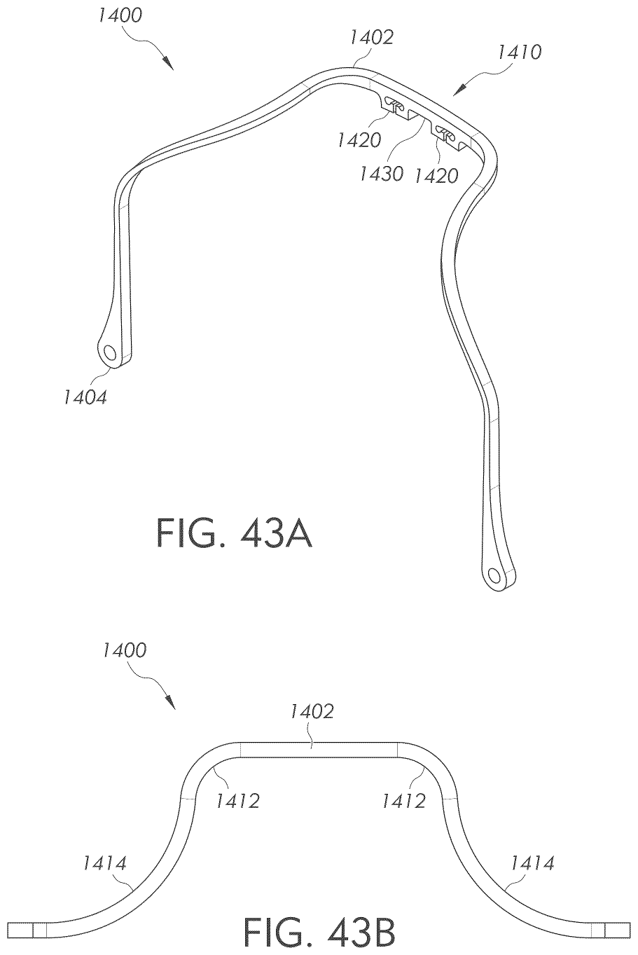

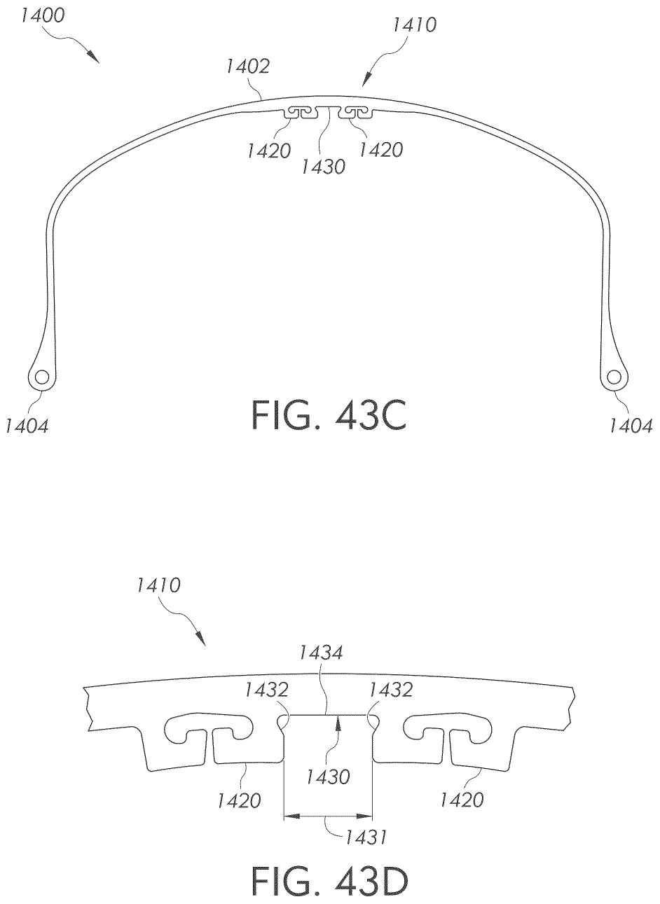

With reference to FIGS. 43A-43D, in some implementations, an outer frame portion 1400 comprises a flex limiting portion 1410. The illustrated flex limiting portion 1410 example includes L-shaped and reverse L-shaped flex limiting components 1420. In some implementations, between the proximal end 1402 and the distal end 1404, the outer frame portions 1400 form a curved shape. For example, in the illustrated example, the shape of the outer frame portion 1400 is illustrated in the expanded state in which the outer frame portions 1400 are wider toward the proximal portion 1402 and narrower toward the distal portion 1404.

In some implementations, the flex limiting portion 1410 can include a clip/connector region 1430 between the components 1420. In some implementations, the paddle frame clip/connector (see FIGS. 31-39) can be disposed within the clip/connector region 1430 between the component 1420. In some implementations, the clip/connector region 1430 can have a width 1431 that is substantially the same as the width of the paddle frame clip/connector or the width of the clip/connector region 1430 can be wider than the pedal frame clip/connector, such that one or more gaps are formed between the paddle frame clip/connector and the flex limiting components. As such, the paddle frame clip/connector and the flex limiting components 1420 can combine to form the flex limiting portion 1410.

The clip/connector region 1430 can have a variety of cross-sectional shapes, for example rectangular, square, trapezoidal, and irregular. In some implementations, the clip/connector region 1430 has a cross sectional shape to match the cross-sectional shape of the paddle frame clip/connector, such that the paddle frame clip/connector can be coupled with the outer frame portion 1400 at the clip/connector region 1430.

With reference to FIG. 43B, the outer frame portions 1400 can include a first curve 1412 and a second curve 1414. With reference to FIG. 43B, the first curve has a radius less than the radius of the second curve 1414, although in some implementations, the first curve 1412 can have a radius greater than or equal to that of the second curve 1414.

Referring to FIG. 43D, the clip/connector region 1430 can include segments 1432 that extend to a surface 1434 of the clip/connector region. In some implementations, the segments 1432 can be substantially perpendicular to the surface 1434 or can be set at angles. In some implementations, the segments 1432 are angled between 45 degrees and 90 degrees relative to the surface 1434. The angled segments 1432 can allow inner ones of the flex components 1420 to flex to allow the clip/connector region 1430 to accept a range of sizes of clips/connectors.

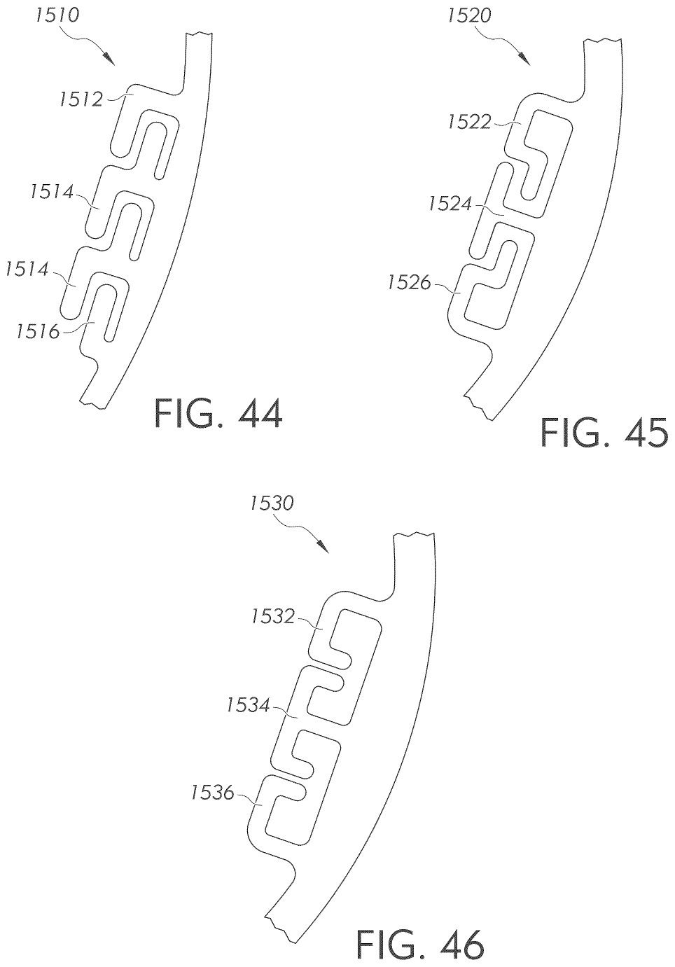

With reference to FIGS. 44-48, 49A and 49B, various examples of flex limiting portions of the outer frame portion are illustrated. As shown in FIGS. 44-48, 49A, and 49B, the flex limiting portions 1510, 1520, 1530 can include cutouts, channels, holes, and/or openings, etc.

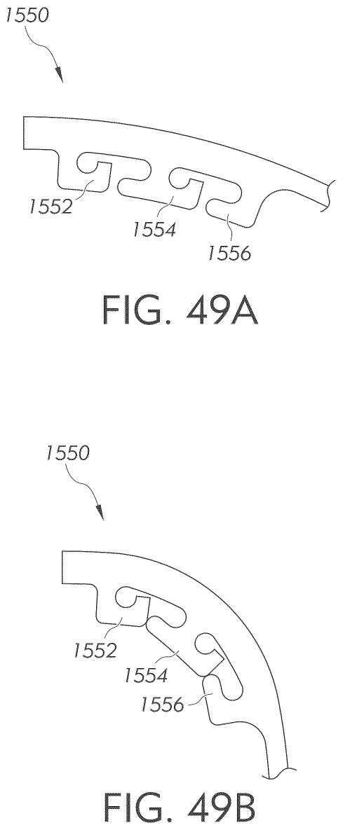

Flex limiting portions with a wide variety of different configurations can operate in the same or substantially the same manner as the flex limiting components 1420 illustrated by FIGS. 43A-43D. For example, the flex limiting members 1512, 1514, 1516 (e.g., stops, extensions, buttresses, etc.) of FIG. 44, the flex limiting members 1522, 1524, 1526 of FIG. 45, the flex limiting members 1532, 1534, 1536 of FIG. 46, and the flex limiting members 1552, 1554, 1556 of FIG. 49A can all engage in the same or similar manner as the flex limiting components 1420 of FIGS. 43A-43D.

In some implementations, upon bending of the outer frame portion, the flex limiting portion can reconfigure or bend until such time that the flex limiting portion engages to stop any further bending of the portion. The flex limiting members can engage simultaneously or sequentially. In some implementations, once the flex limiting portion engages and stops flexing, other portions of the paddle frame can bend or flex or continue to bend or flex. As such, the flex limiting portions can be used to control the flexing or bending of the outer frame portion. In some implementations, as the implantable valve repair device is narrowed, the flex limiting portion limits bending. In some implementations, the flex limiting portion allows other portions of the paddle frame to bend after the stop of the flex limiting portion has been reached.

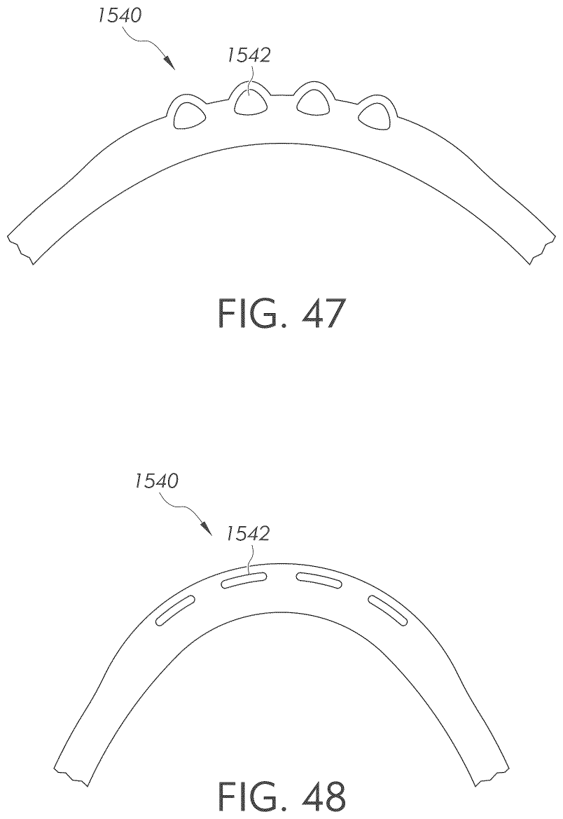

In the example illustrated by FIGS. 47-48, the flex limiting portion 1540 can include one or more openings or holes 1542 in the outer frame portion. In some implementations, upon bending of the outer frame portion, the flex limiting portion 1540 can reconfigure or bend until such time that the holes or openings flatten out to stop any further bending of the flex limiting portion 1540. In some implementations, once the flex limiting portion stops flexing, other portions of the paddle frame can bend or flex. As such, the flex limiting portions illustrated by FIGS. 47 and 48 can be used to control the flexing or bending of the outer frame portion. In some implementations, the flex limiting portion allows other portions of the paddle frame to bend after holes of the flex limiting portion have flattened out.

As illustrated in FIGS. 49A-B, the flex limiting portion 1550 includes the flex limiting members 1552, 1554, 1556. The flex limiting members 1552, 1554, 1556 and spaces between the flex limiting members can have a wide variety of different sizes and shapes that can be selected to control the bending of the flex limiting portion 1550. The flex limiting portion 1550 of FIG. 49A is illustrated in an unflexed or normal state. Referring to FIG. 49B, the flex limiting portion 1550 is shown bent such that the flex limiting members 1552, 1554, 1556 engage one another and stop further bending of the flex limiting portion 1550.

Referring back to FIGS. 38A and 38B, the illustrated example valve repair device 900 includes an outer frame portion 940 with an optional flex limiting portion 960. In some implementations, the flex limiting portion 960 includes a pair of inner flex limiting members 962 (e.g., stops, extensions, buttresses, etc.) and a pair of outer flex limiting members 964 (e.g., stops, extensions, buttresses, etc.). In some implementations, the inner flex limiting portions 962 are connected to the inner frame portion 930 by pivot connections 946, such as by pins or other fasteners that allow pivotal movement.

In some implementations, when outer frame portion 940 is narrowed, the inner flex limiting portions 962 pivot about the pivot connections 946 and the first and second portions 942, 944 of the outer paddle frame portion 940 flex until the inner flex limiting portions 962 engage the outer flex limiting portions 964. Further narrowing of the outer paddle frame portion can cause rotation of the engaged inner and outer flex limiting portions 962, 964 about the pivot connections 946 and/or cause other portions of outer paddle frames 940 to flex.

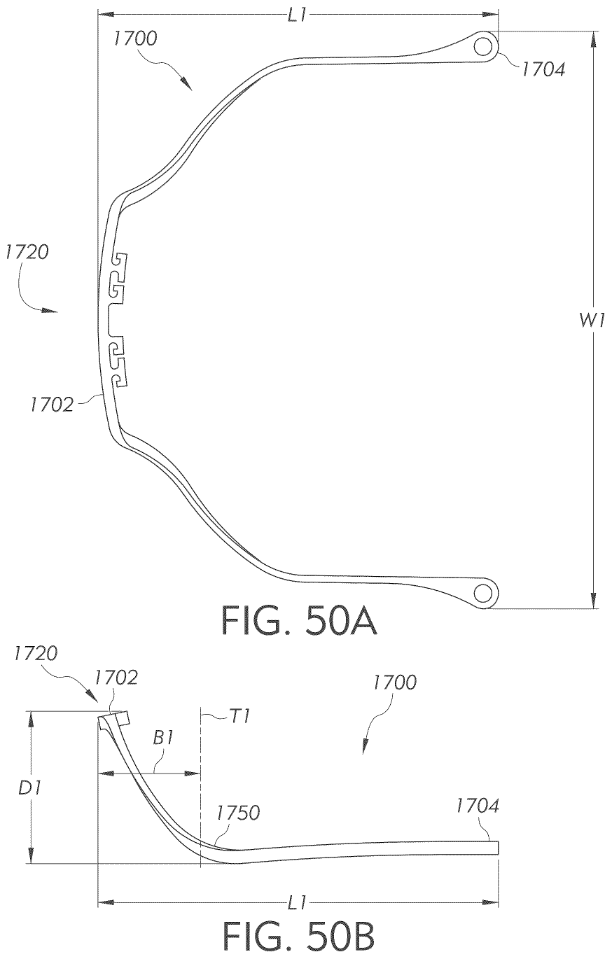

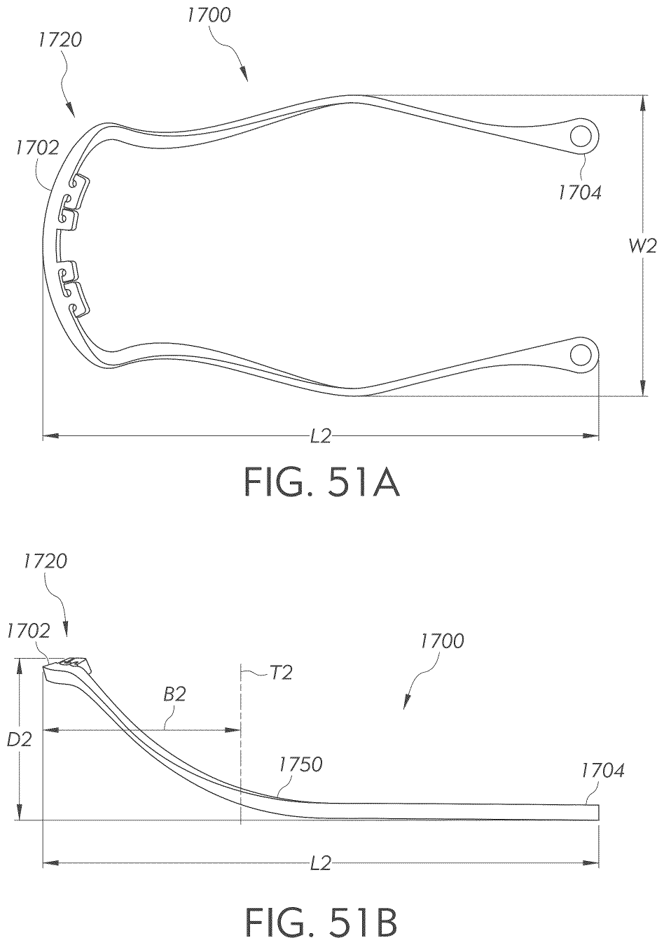

Referring now to FIGS. 50A-50B, an example outer frame portion 1700 comprising an optional flex limiting portion 1720 is illustrated in an expanded or wide state. The outer frame portion 1700 has a width W1, a length L1, and a depth D1. With reference to FIGS. 51A-51B, the outer frame portion 1700 with the optional flex limiting portion 1720 is illustrated in a narrowed state. The outer frame portion 1700 in the narrowed state comprises a width W2, a length L2, and a depth D2.

In some implementations, as the outer frame portion 1700 is narrowed from the expanded state to the narrowed state, the width and depth decrease, while the length, which is measured from the proximal end 1702 to the distal end 1704, increases.

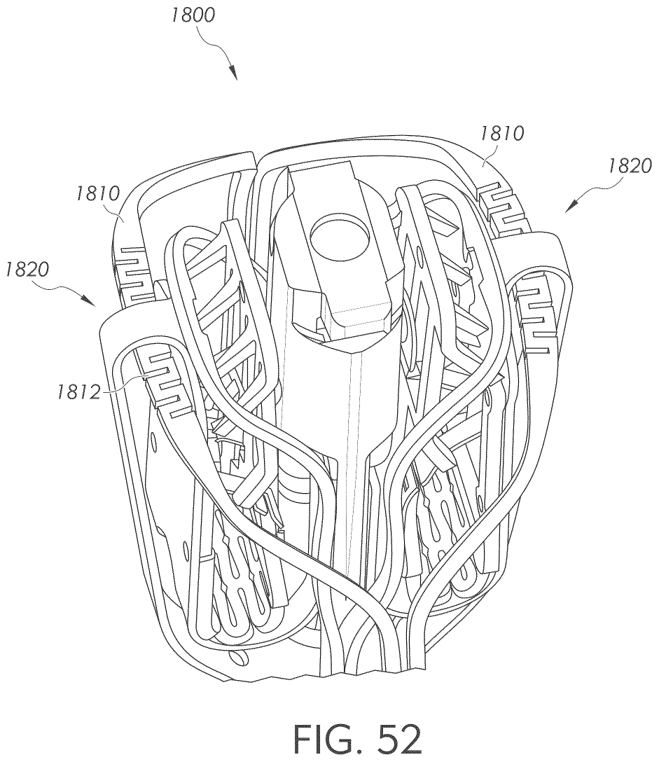

With reference to FIG. 52, an example implantable valve repair device 1800 is illustrated. The implantable valve repair device 1800 comprises an outer frame portion 1810 comprising a flex enhancing portion 1820. The outer frame portion 1810 comprises slits 1812 on alternating sides of the outer frame portion 1810, each slit extending towards the center of the flex enhancing portion 1820 of the outer frame portion 1810. In some implementations, as illustrated, the slits create an undulating, zigzag or serpentine flex enhancing portion. The number, orientation, depth, width, etc. of the slits 1812 of the outer frame portion 1810 can be selected to control the flexibility of a flex enhancing portion 1820 of outer frame portion 1810.

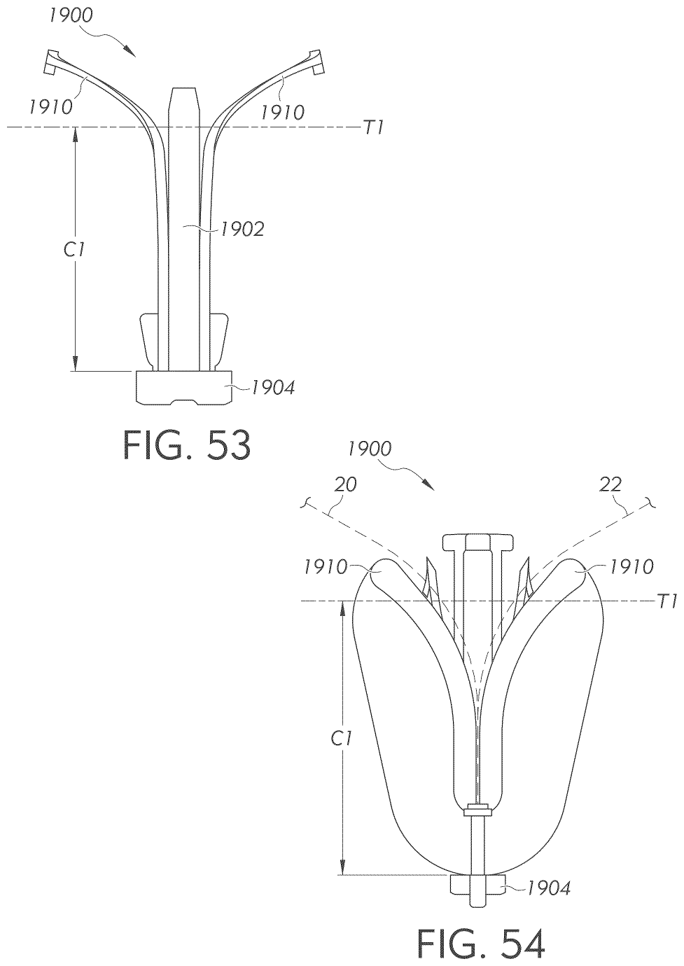

With reference to FIGS. 53-56, an example implantable valve repair device 1900 is illustrated, with various components omitted or shown generally to simplify the drawing. The implantable valve repair device 1900 comprises an outer frame portion 1910, an optional spacer or coaptation element 1902, and a base or cap 1904. In some implementations, the implantable valve repair device 1900 can comprise an optional paddle frame clip/connector coupled to at least one paddle frame portion (e.g., the inner paddle frame portion or the outer paddle frame portion) and at least one paddle (e.g., the outer paddle or the inner paddle) (see FIGS. 31-42).

In some implementations, the implantable valve repair device 1900 can comprise an optional flex limiting portion on the outer frame portion 1910 (FIGS. 43A-52). In some implementations, the implantable valve repair device 1900 comprises both the optional flex limiting portion on the outer frame portion 1910 and one or more optional paddle frame clips/connectors.

With reference to FIGS. 54 and 56, the implantable valve repair device 1900 can be configured to capture one or more leaflets. In some implementations, when two leaflets are being captured and the implantable valve repair device 1900 is deployed and closed, the leaflets can be brought together, pressed together, or coapted by the paddle frames 1910.

In some implementations, the paddle frames 1910 can be configured to bring the leaflets together along a majority of a height of the device. In the illustrated example, the leaflets are first brought together or substantially brought together by the paddle frames 1910 at a paddle frame coaptation position T1, T2. In some implementations, the paddle frames 1910 are configured to hold the leaflets together from the paddle frame coaptation position T1, T2 to the depth to which the leaflets extend in the device.

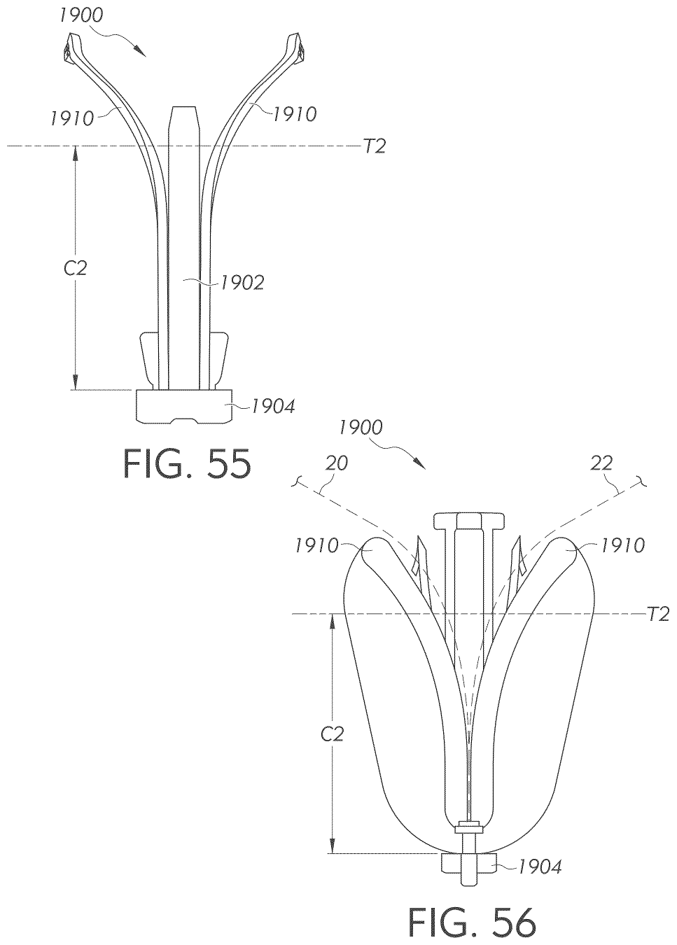

The paddle frames 1910 are shown in a wide or substantially wide configuration in FIGS. 53 and 54 and the paddle frames are shown in a narrower configuration in FIGS. 55 and 56. In some implementations, in the wide or substantially wide configuration, the leaflets 20, 22 can first be pressed together by the paddle frames 1910 of the implantable valve repair device 1900 at the paddle frame coaptation position T1, which is distance C1 from the base or cap 1904.

The implantable valve repair device 1900 illustrated in FIGS. 55-56 is in a narrowed or partially narrowed state. In some implementations, in the partially narrowed state, the leaflets 20, 22 can first be pressed together by the paddle frames of the implantable valve repair device 1900 at coaptation position T2, which is a distance C2 from the base or cap 1904.

In some implementations, the paddle frames 1910 can be moved from a completely widened configuration (i.e., 0% narrowed) where the outer paddle frame portions extend as far away from the inner paddle frame portions to a completely narrowed configuration (i.e., 100% narrowed) where the outer paddle frame portions are the same width or substantially the same width as the inner paddle frame portions or are as close as possible to the inner paddle frame portions.

In some implementations, the outer paddle frame portions can be configured so that the paddle frame coaptation position T2 (such as when the paddle frames 1910 are between about 50% and 95% narrowed) and the paddle frame coaptation position T1 (such as when the paddle frames 1910 are 0% narrowed) are close together. For example, the distance C2 when the paddle frames are between 50% and 95% or any subrange narrowed can be between 70% and 100% or any subrange of the distance C1 that corresponds to the paddle frames being 0% narrowed.

The coaptation position can depend on many factors, including but not limited to the anatomy of the leaflets, the positioning of the implantable valve repair device, and the bending of the paddle frames of the implantable valve repair device during expanding and narrowing. A more distal paddle frame coaptation position along the spacer or coaptation element 1902 (i.e., a position closer to the base or cap 1904-a shorter distance C1 or C2) can result in less of the leaflet 20, 22 being pressed together by the paddle frames 1910 of the implantable valve repair device 1900.

The valve repair device can be configured in a wide variety of different ways to keep the distance C2 close to the distance C1. In some implementations, the outer paddle frame portions are configured to keep the distance C2 close to the distance C1. For example, the paddle frame portions can be configured to keep the distance C2 between 70% and 100% of the distance C1. In this example, C2 can correspond to the paddle frames being between 50% and 95% or any subrange narrowed and C1 can correspond to the paddle frames being 0% narrowed. For example, the outer paddle frame portions 1400, 1700 or features of the paddle frame portions 1400, 1700 can be used to keep the distance C2 between 70% and 100% of the distance C1. In this example, C2 can correspond to the paddle frames being between 50% and 95% or any subrange narrowed and C1 can correspond to the paddle frames being 0% narrowed.

In some implementations, a variety of features and/or configurations can be included to help to increase the distance C2 between the paddle coaptation position T2 and the base or cap 1904. For example, the shape of the outer paddle frame portion, the optional paddle frame clip(s) and/or the optional flex limiting portion can be configured to increase the distance C2 between the paddle coaptation position T2 of the narrowed paddle frames. As a result, the amount of leaflet 20, 22 pressed together by the narrowed paddle frames is increased.

In some implementations, the distance C2 between the coaptation position T2, and the base or cap 1904 can be substantially the same as the distance C1 between the coaptation position T1, and the base or cap 1904 (FIGS. 53-54). In some implementations, the distance C2 between the coaptation position T2, and the base or cap 1904 can be between 0.0 mm-1.0 mm or any subrange less than the distance C1 between the coaptation position T1, and the base or cap 1904. For example, in some implementations, the distance C2 between the coaptation position T2, and the base or cap 1904 can be between 0.25 mm-0.75 mm less than the distance C1 between the coaptation position T1, and the base or cap 1904.

Referring back to FIGS. 50B and 51B, the outer frame portions 1700 can include a bend 1750 that corresponds to the coaptation positions T1, T2. In some implementations, the outer frame portion 1700 is configured to keep the bend 1750 and thus the paddle frame coaptation positions T1, T2 close as the frame is moved from the wide configuration to narrow configurations. In FIG. 50B, a distance B1 is the distance from a proximal end to the bend 1750 when the outer paddle frame portion is in the wide configuration. In FIG. 51B, a distance B2 is the distance from a proximal end to the bend 1750 when the outer paddle frame portion is in a narrowed configuration.

In some implementations, the outer paddle frame portion 1700 can be configured to keep the distance B2 between 70% and 100% of the distance B1. In this example, B2 can correspond to the paddle frames being between 50% and 95% or any subrange narrowed and B1 can correspond to the paddle frames being 0% narrowed.

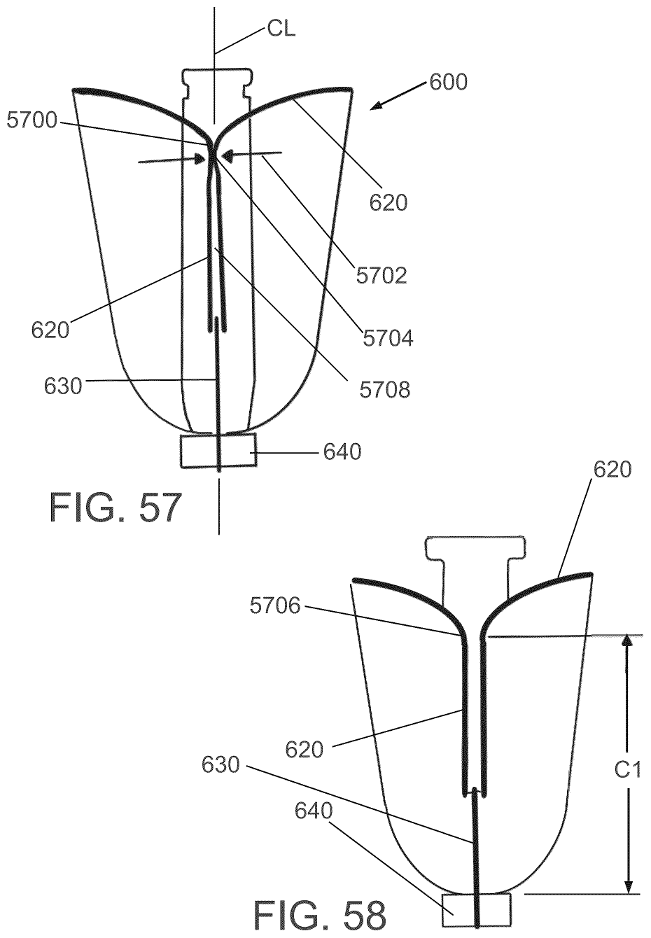

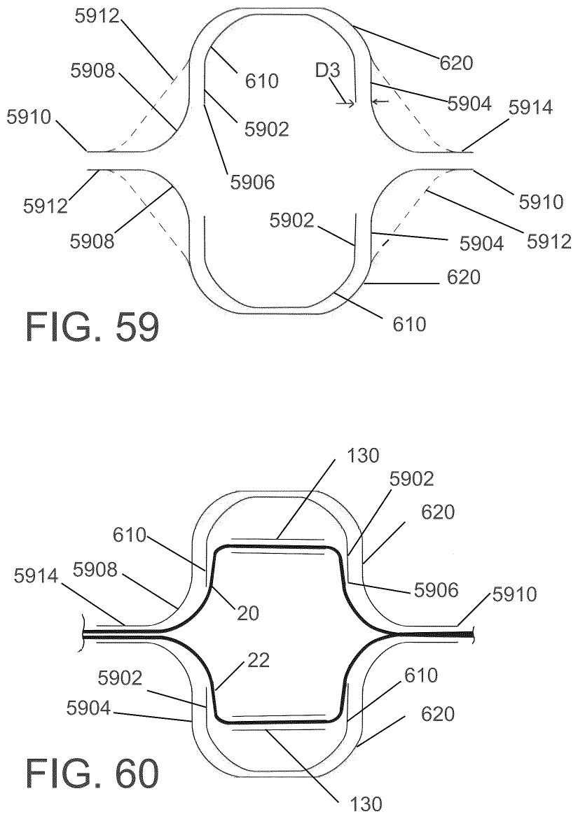

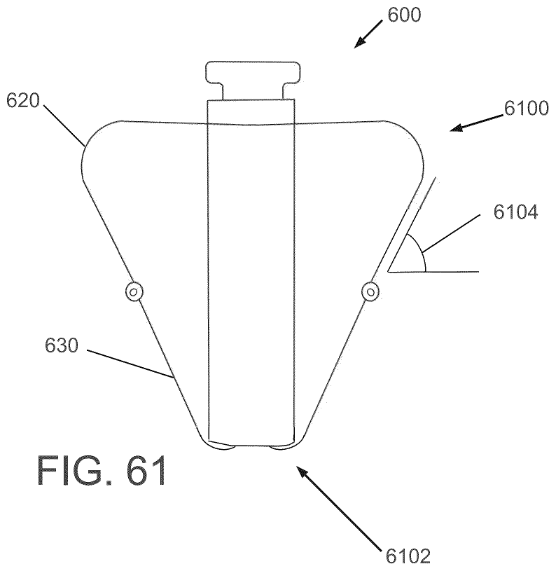

Referring to FIGS. 57-61, in some implementations the outer frame portions 620 can be configured to enhance leaflet engagement by the valve repair device 600. For example, the outer frame portions 620 can be configured to pinch together (see FIG. 57), the outer frame portion 620 can be configured to maximize the shoulder height (see FIG. 58), the outer frame portion 620 can wrap around the inner frame portion (see FIGS. 59 and 60) and/or the outer frame portion 620 can be tapered (see FIG. 61) to enhance the efficacy of the valve repair device 600.

Referring to FIG. 57, the outer frame portions 620 can be configured to pinch together in a variety of different ways. For example, the outer frame portions 620 can be shapeset to provide a pinch point, the outer frame portions can be biased together by a spring or other resilient material, etc.

In some implementations, the outer frame portions 620 can be shapeset such that one or more portions 5700 of each outer frame portion 620 is biased past a center line CL. In some implementations, when the two outer frame portions 620 are assembled in the device, the one or more portions 5700 engage one another at the center line CL. In some implementations, the biasing force past the center line CL creates a pinching force 5702 where the two outer frame portions 620 meet.

In the example illustrated by FIG. 57, a discrete pinch point 5704 is formed between the two outer frame portions 620 at or near the shoulders 5706 (see FIG. 58) of the two outer frame portions 620 and there is a space or gap 5708 between the rest of the outer frame portions 620. However, the outer frame portions 620 can be shape set such that a plurality of pinch points 5704 are formed and/or such that one or more elongated pinch area 5704 is formed. In some implementations, the outer frame portions can be configured to provide an elongated pinch force along the outer frame portions from the connector 630 to shoulders 5706.

With reference to FIG. 58, in some implementations the outer frame portion 620 can be configured to maximize the shoulder height C1. The implantable valve repair device 600 can capture one or more leaflets (e.g., leaflet 20, 22). When multiple leaflets are captured, the leaflets can be brought or pinched together by the paddle frames 620 as described with respect to FIG. 59. The paddle frames 620 can be configured to bring the leaflets together at the shoulder 5706. In some implementations, the outer frames 620 are configured to hold the leaflets together from the shoulder 5706 to the depth to which the leaflets extend in the device.

In some implementations, the outer frame portions 620 are configured such that the difference between the shoulder height C1 in the wide position and the shoulder height C2 (see FIG. 56) in the narrow position is minimized. In the wide or substantially wide configuration, the leaflets 20, 22 are pressed together by the outer paddle frame portions 620 at a distance C1 from the base or cap 640.

In some implementations, the outer frame portions 620 can be moved from a completely widened configuration (i.e., 0% narrowed) where the outer paddle frame portions extend as far away from the inner paddle frame portions to a completely narrowed configuration (i.e., 100% narrowed) where the outer paddle frame portions are the same width or substantially the same width as the inner paddle frame portions.

In some implementations, the outer paddle frame portions 620 can be configured so that the shoulder height C2 (such as when the paddle frames 1910 are between about 50% and 95% narrowed) and the shoulder height C1 (such as when the paddle frames 1910 are 0% narrowed) are close together. For example, the distance C2 when the paddle frames are between 50% and 95% or any subrange narrowed can be between 70% and 100% or any subrange of the distance C1 that corresponds to the paddle frames being 0% narrowed.

The valve repair device can be configured in a wide variety of different ways to keep the distance C2 close to the distance C1. In some implementations, the outer frame portions 620 are shape set to keep the distance C2 close to the distance C1 and such that the outer frame portions 620 pinch together at the shoulder 5706 as illustrated by FIG. 57. For example, the paddle frame portions can be shape set to keep the distance C2 between 70% and 100% of the distance C1. In this example, C2 can correspond to the paddle frames being between 50% and 95% or any subrange narrowed and C1 can correspond to the paddle frames being 0% narrowed. For example, the outer paddle frame portions 620 can be shape set to keep the distance C2 between 70% and 100% of the distance C1. In this example, C2 can correspond to the paddle frames being between 50% and 95% or any subrange narrowed and C1 can correspond to the paddle frames being 0% narrowed.

In some implementations, shoulder height C2 can be substantially the same as the shoulder height C1. In some implementations, the shoulder height C2 can be between 0.0 mm-1.0 mm or any subrange less than the shoulder height C1. For example, in some implementations, the shoulder height C2 can be between 0.25 mm-0.75 mm less than the shoulder height C1.

Referring to FIGS. 59 and 60, the outer frame portions 620 can wrap around the inner frame portions 610 when viewed from above (i.e., from the proximal end). Wrapping the outer frame portions 620 around the inner frame portions 610 when viewed from above or the proximal end can help to constrain the paths of the outer surfaces (ventricular sides) of the valve leaflets 20, 22. For example, referring to FIG. 60, the valve leaflets 20, 22 are shown engaged by clasps 130 between sides 5902 of the inner frame portions 610. The sides 5902 of the inner frame portions 610 guide the valve leaflets 20, 22 from the clasps 130 toward one another. In some implementations, the outer frame portions 620 are configured to wrap around the inner frame portions 610 when viewed from above (i.e., from the proximal end), while also pinching the leaflets together as shown in FIG. 57 and/or maximizing the shoulder height C1 as shown in FIG. 58.

In some implementations, when viewed from above or the proximal end, sides 5904 of the outer frame portions 620 are close to and optionally parallel to the sides 5902 of the inner frame portions 610. For example, the sides 5904 of the outer frame portions 620 can be a distance D3 from the sides 5902 of the inner frame portions 610. The distance D3, when viewed from above or the proximal end, can be between 0.01 mm and 0.5 mm, such as between 0.01 mm and 0.2 mm, such as between 0.01 mm and 0.1 mm, or any subrange of these ranges.

In some implementations, when viewed from above or the proximal end, the sides 5904 of the outer frame portions 620 are parallel to the sides 5902 of the inner frame portion at least to ends 5906 of the inner frame portions 610. In some implementations, when viewed from above or the proximal end, the sides 5904 of the outer frame portions 620 are parallel to the sides 5902 of the inner frame portion past the ends 5906 of the inner frame portions 610 or slightly short of the ends 5906 of the sides of the inner frame portions 610.

In some implementations, when viewed from above or the proximal end, the outer frame portions 620 curves away from the inner frame portions 610 at or near the ends 5906 of the sides 5902 of the inner frame portion 610 at curved portions 5908. For example, the outer frame portions 620 can begin to curve away from the inner frame portions 610 within 0.5 mm of the ends 5906, within 0.3 mm of the ends 5906, within 0.1 mm of the ends 5906, or within 0.05 mm of the ends 5906.

In some implementations, the curved portions 5908 guide the leaflets 20, 22 both toward one another and toward sides 5910 of the outer frame portions. Referring to FIG. 59, the dashed lines 5912 represent an area where the leaflets 20, 22 could extend without the curved portions 5908. The leaflets 20, 22 could bunch up or buckle and/or could be spaced apart from one another in this area. In some implementations, the curved portions 5908 constrain the paths of the outer surfaces (ventricular sides) of the valve leaflets 20, 22 to bring the leaflets together and/or inhibit bunching up or buckling of the leaflet. In some implementations, the curved portions 5908 extend to a straight portion 5914 where are the leaflets 20, 22 are brought together when viewed from above or an atrial side. The straight portions 5914 extend from the curved portions 5908 to the sides 5910 of the outer frame portions 620.

In some implementations, the outer frame portion 620 and/or the connector 630 can be tapered or reduced in width from the proximal end 6100 to the distal end 6102 when viewed from the front or rear, as shown in FIG. 61. As is described in detail herein, the outer frame portions 620 can be moved between narrow and wide configurations. The narrow configuration allows the device 600 to be maneuvered through the patient's anatomy. For example, the narrowed outer frame portions 620 and attached paddles can be routed through the chordae tendineae CT to allow the paddles to be positioned on the outside or ventricular side of the leaflets 20, 22. By configuring the outer frame portions 620 and/or the connector 630 to be tapered in the wide position, the connector and/or the distal end of the outer frame portions 620 will still fit between the chordae tendineae CT in the wide position. Further, referring FIG. 3A the tapered shape of the outer frame portions 620 can more closely approximate the tapered shape of the native valve leaflets 20, 22.

The outer frame portions 620 can be configured to taper from the proximal end 6100 to the distal end 6102 in a variety of different ways. For example, the outer frame portions 620 can be shapeset to taper from the proximal end 6100 to the distal end 6102. In some implementations, the taper angle 6104 can be between 30 and 80 degrees, such as between 45 and 80 degrees, such as between 60 and 75 degrees, or any sub range of these ranges.

In some implementations, the taper of the outer frame portions 620 is continuous with the taper of the connector 630. For example, the angle 6104 of the taper of the outer frame portions can be the same as the angle of the taper of the connector 630. In some implementations, the angle 6104 of the taper of the outer frame portions is different than the angle of the taper of the connector 630 or the connector is not tapered.

In some implementations, the outer frame portions 620 are tapered while also being configured to wrap around the inner frame portions 610 when viewed from above (i.e., from the proximal end) as shown in FIGS. 59 and 60, being configured to pinch the leaflets together as shown in FIG. 57 and/or maximizing the shoulder height C1 as shown in FIG. 58.

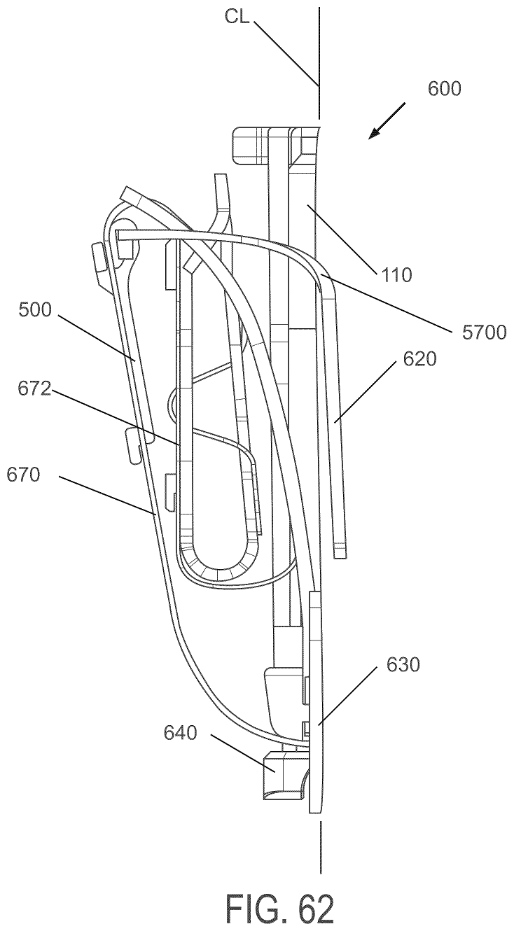

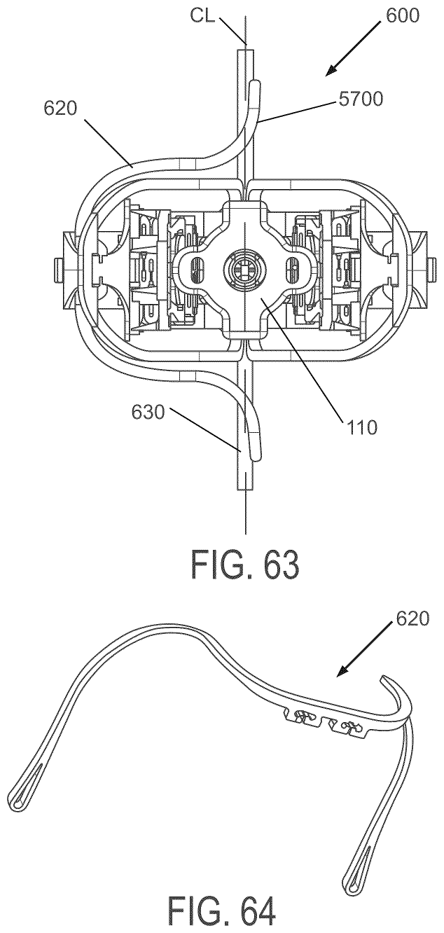

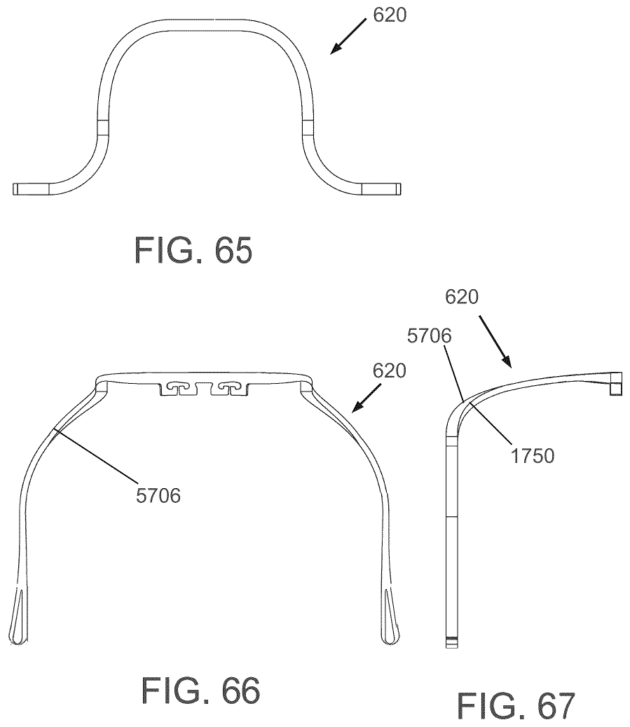

FIGS. 62-71 illustrate an example of a valve repair device 600. Referring to FIG. 62, in some implementations the outer frame portions 620 can be configured to pinch together by shape setting of the outer frame portions 620. In some implementations, the outer frame portion 620 is sized and shaped set such that that the outer frame portion 620 extends past the centerline CL when the outer frame portion 620 is attached to the outer paddle 670, but is not yet attached to the connector, when viewed from the top or the side. For example, the outer frame portion 620 can be sized and shaped such that the outer frame portion 620 extends between 0.2 mm and 3 mm, between 0.3 mm and 2.5 mm, between 0.4 mm and 2.0, between 0.5 mm and 1.5 mm, or any sub-range in these ranges past the centerline CL. To finish assembly of the outer frame portions 620 to the valve repair device, the outer frame portions 620 are stretched and connected to the connector 630.

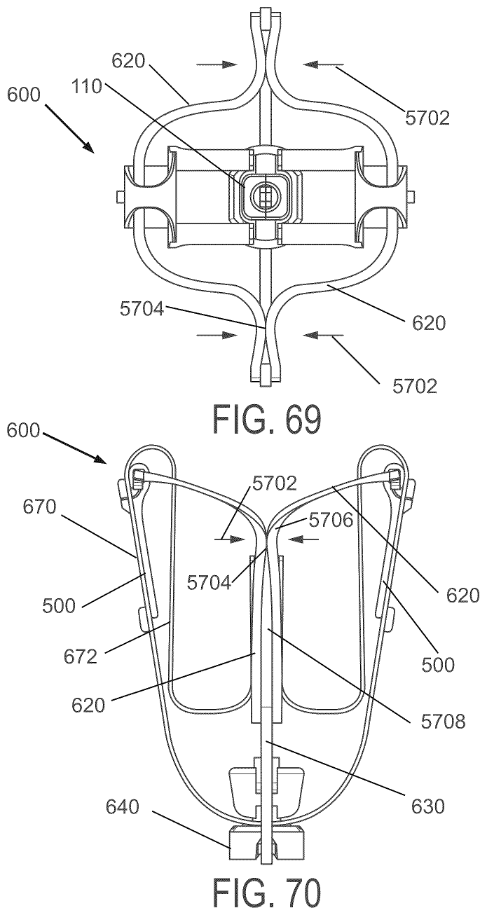

Referring to FIGS. 69 and 70, once the two outer frame portions 620 are assembled with the clip/connector 500 and the connector 630, the two outer frame portions provide a pinch point 5704. In some implementations, the outer frame portions 620 can be shapeset such that one or more portions 5700 of each outer frame portion 620 is biased past a center line CL. In some implementations, when the two outer frame portions 620 are assembled in the device, the one or more portions 5700 engage one another at the center line CL. In some implementations, the biasing force past the center line CL creates a pinching force 5702 where the two outer frame portions 620 meet. In the example illustrated by FIGS. 69 and 70, a discrete pinch point is formed between the two outer frame portions 620 at or near the shoulders 5706 (see FIG. 69) of the two outer frame portions 620 and there is a space or gap 5708 between the rest of the outer frame portions 620.

However, the outer frame portions 620 can be shape set such that a plurality of pinch points 5704 are formed and/or such that one or more elongated pinch area is formed. In some implementations, the outer frame portions can be configured to provide an elongated pinch force along the outer frame portions from the connector 630 to the shoulders 5706.

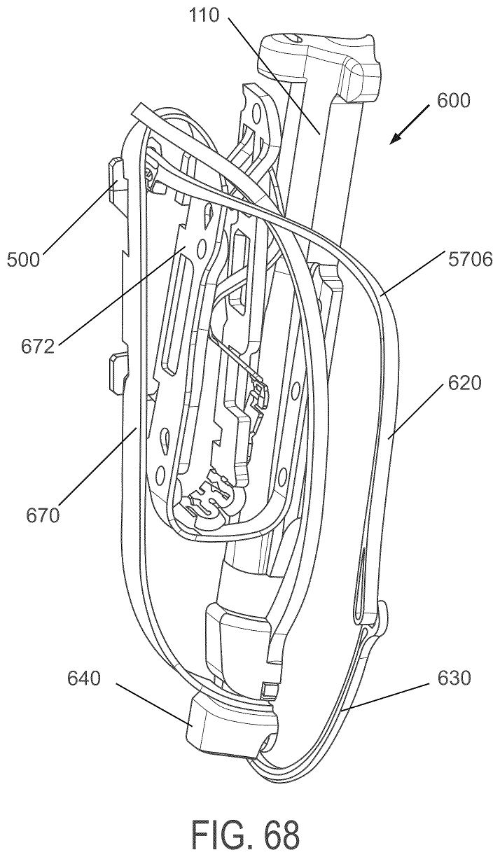

With reference to FIGS. 68-71, an implantable valve repair device 600 is illustrated, with various components omitted or shown generally to simplify the drawing. The implantable valve repair device 600 comprises an outer frame portion 620, such as the outer frame portion illustrated by FIGS. 62-67, an optional coaptation element 110 (e.g., a spacer, coaption element, plug, gap filler, etc.), and a base or cap 640. In some implementations, the implantable valve repair device 600 can comprise one or more optional paddle frame clips/connectors 500 coupled to at least one paddle frame portion (e.g., the inner paddle frame portion 610 or the outer paddle frame portion 620) and at least one paddle (e.g., the outer paddle 670 or the inner paddle 672). In some implementations, the implantable valve repair device 600 can comprise an optional flex limiting portion on the outer frame portion 620. In some implementations, the implantable valve repair device 600 comprises both the optional flex limiting portion on the outer frame portion 620 and one or more of the optional paddle frame clips/connectors 500.

The implantable valve repair device 600 can be configured to capture one or more leaflets. When multiple leaflets are being captured and the implantable valve repair device 600 is deployed and closed, the leaflets can be brought together, pressed together, or coapted by the outer paddle frame portions 620. The outer paddle frame portions 620 can be configured to bring the leaflets together along a majority of a height of the device. In the illustrated example, the leaflets are first brought together or substantially brought together by the outer frame portions 620 at a shoulder 5706.

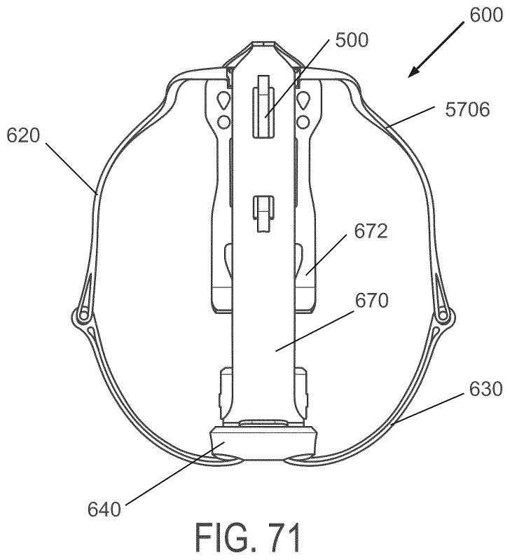

The outer frame portions 620 are shown in a wide or substantially wide configuration in FIG. 71. In some implementations, in the wide or substantially wide configuration, the leaflets 20, 22 are first pressed together by the outer paddle frame portions 620 of the implantable valve repair device 600 at the shoulder 5706, which is distance C1 from the base or cap 640 (see FIG. 58—the distance C1 is also the distance from the shoulder to the base or cap in FIG. 70).

In some implementations, the outer frame portions 620 can be moved from a completely widened configuration (i.e., 0% narrowed) where the outer paddle frame portions extend as far away from the inner paddle frame portions to a completely narrowed configuration (i.e., 100% narrowed) where the outer paddle frame portions are the same width or substantially the same width as the inner paddle frame portions or are as close as possible to the inner paddle frame portions.

In some implementations, the outer paddle frame portions 620 can be configured so that the shoulder 5706 (such as when the paddle frames 1910 are between about 50% and 95% narrowed) and the shoulder 5706 (such as when the paddle frames 1910 are 0% narrowed) are close together. For example, the distance from the cap 640 to the shoulder when the paddle frames are between 50% and 95% or any subrange narrowed can be between 70% and 100% or any subrange of the distance C1 to the shoulder that corresponds to the paddle frames being 0% narrowed.

Referring to FIGS. 64-67, the outer frame portions 620 can include a bend 1750 that corresponds to the shoulder 5706. In some implementations, the outer frame portion 1700 is configured to keep the bend 1750 and thus the positions of the shoulder 5706 close as the frame is moved from the wide configuration to narrow configurations. In some implementations, the outer paddle frame portion 620 can be configured to keep the distance from the cap to the shoulder between 70% and 100% of the distance C1. In this example, the distance from the cap to the shoulder can correspond to the paddle frames being between 50% and 95% or any subrange narrowed and the distance C1 can correspond to the paddle frames being 0% narrowed.

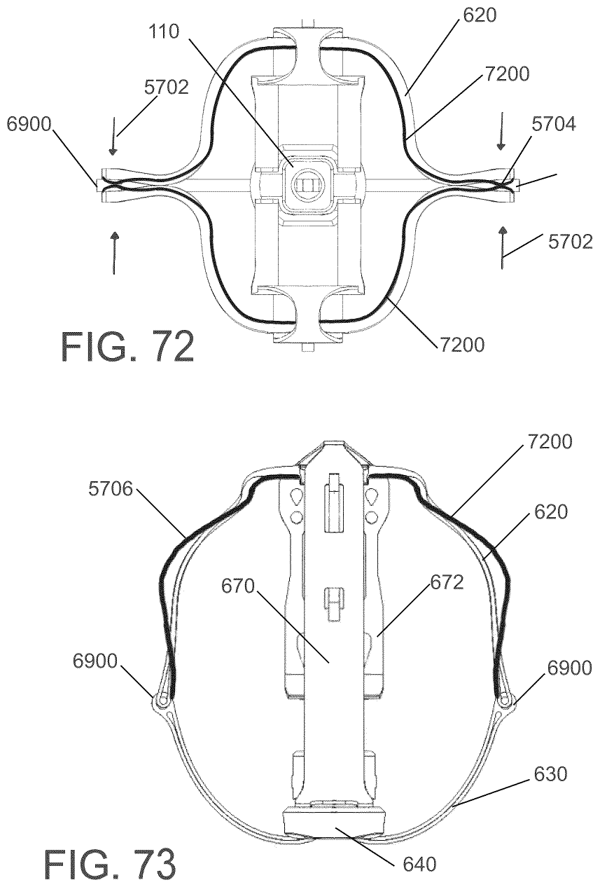

Referring to FIG. 69, in some implementations, the pinch points 5704 are inward of the ends 6900 of the connector 630, when viewed from the top or a proximal end. Referring to FIGS. 72 and 73, in some implementations the pinch points 5704 are substantially aligned with the ends 6900 of the connector 630, when viewed from the top or a proximal end. For example, the pinch points 5704 can be aligned to within 0.5 mm, such as within 0.4 mm, such as within 0.3 mm, or such as within 0.2 mm, when viewed from the top or a proximal end.

The pinch points 5704 can be substantially aligned with the ends 6900 of the connector 630 in a variety of different ways. For example, the outer frame portion 620 can be shape set to move the pinch points 5704 to be substantially aligned with the ends 6900 of the connector 630, when viewed from the top or proximal end of the device 600. Referring to FIGS. 72 and 73, a dark black line 7200 represents a modified shape of the outer frame portion 620. The modified shape 7200 has a shoulder 5706 that is moved outward or laterally to move the pinch points 5704 into substantial alignment with the ends 6900 of the connector 630. However, the pinch points can be moved outward or laterally in any manner.

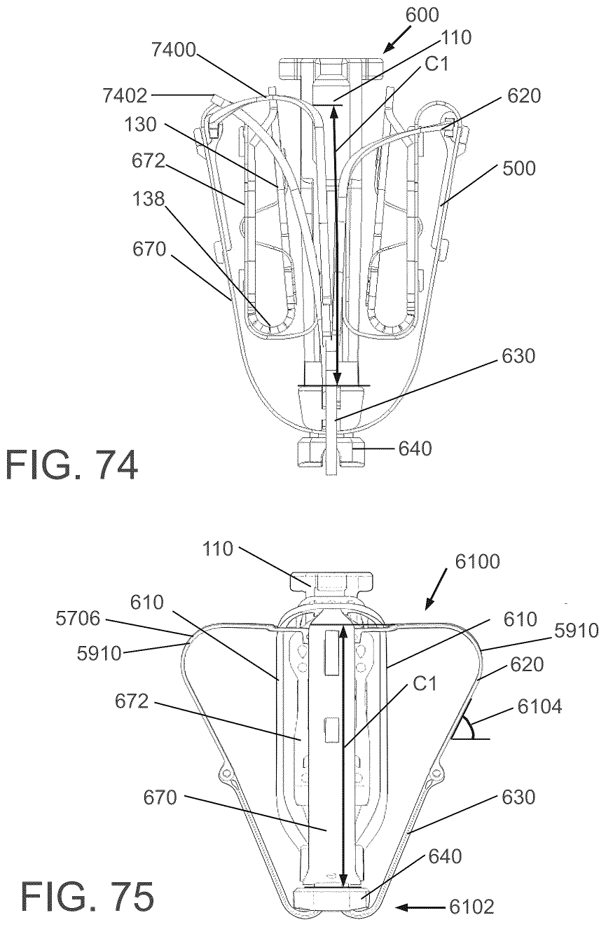

FIG. 74 is an illustration where the outer frame portion 620 on the left has an increased shoulder height C1. In some implementations, the device 600 can have the same outer frame portion 620 on both sides of the device, but the outer frame portion 620 on the right does not have the increased shoulder height to call attention to the increased shoulder height of the outer frame portion 620 on the left of the device 600.

The shoulder height C1 can be increased in a variety of different ways. In some implementations, the shoulder height C1 is increased by sizing and shape setting of the outer frame portion 620. In some implementations, a top or most proximal point 7400 of the outer frame portion 620 is substantially aligned with a top or most proximal point 7402 of the inner frame portion 610. For example, the top or most proximal point 7400 of the outer frame portion 620 can be within 0.5 mm, within 0.4 mm, within 0.3 mm, within 0.2 mm, or within 0.1 mm of the top or most proximal point 7402 of the inner frame portion 610, when the device 600 is viewed from the side.

In some implementations, the paddle frames 620 can be configured to bring the leaflets together at the shoulder 5706. In some implementations, the outer frames 620 are configured to hold the leaflets together from the shoulder 5706 to the depth (e.g., to the hinge portion 138 of the clasp 130) to which the leaflets extend into the device. In some implementations, the outer frame portions 620 are configured to substantially align a top or most proximal point 7400 of the outer frame portion 620 with a top or most proximal point 7402 of the inner frame portion 610, while also being tapered, being configured to wrap around the inner frame portions 610 when viewed from above (i.e. from the proximal end), and/or being configured to pinch the leaflets together.

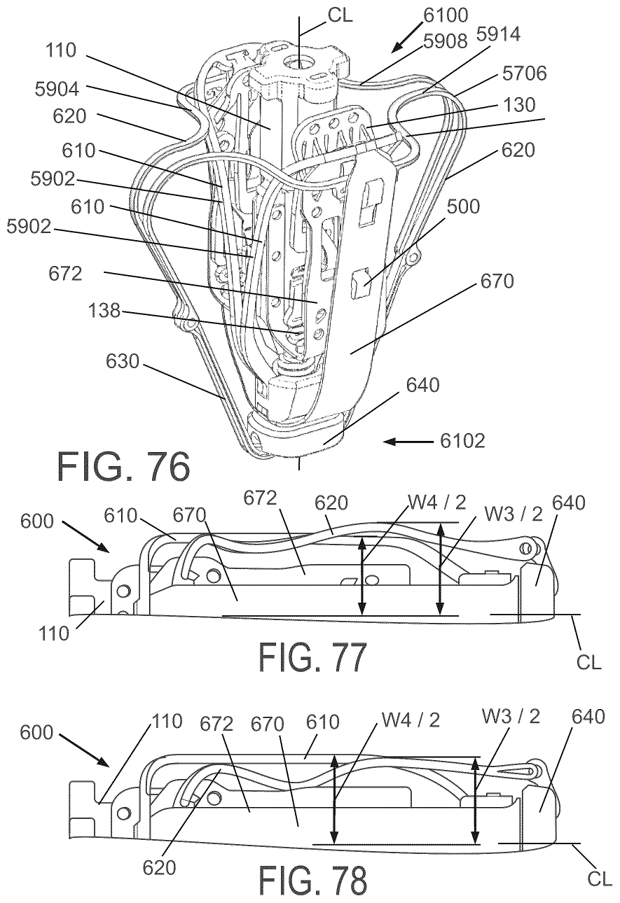

Referring to FIGS. 75 and 76, in some implementations, the outer frame portion 620 and/or the connector 630 can be tapered or reduced in width from the proximal end 6100 to the distal end 6102 when viewed from the front or rear. As is described in detail herein, the outer frame portions 620 can be moved between narrow and wide configurations. The narrow configuration allows the device 600 to be maneuvered through the patient's anatomy. For example, the narrowed outer frame portions 620 and attached paddles can be routed through the chordae tendineae CT to allow the paddles to be positioned on the outside or ventricular side of the leaflets 20, 22. In some implementations, by configuring the outer frame portions 620 and/or the connector 630 to be tapered in the wide position, the connector and/or the distal end of the outer frame portions 620 will still fit between the chordae tendineae CT in the wide position. Further, referring FIG. 3A the tapered shape of the outer frame portions 620 can more closely approximate the tapered shape of the native valve leaflets 20, 22.

Referring to FIG. 75, in some implementations, the taper angle 6104 can be between 30 and 80 degrees, such as between 45 and 80 degrees, such as between 60 and 75 degrees, or any sub range of these ranges. In some implementations, the taper of the outer frame portions 620 is continuous with the taper of the connector 630. For example, the angle 6104 of the taper of the outer frame portions can be the same as the angle of the taper of the connector 630. In some implementations, the angle 6104 of the taper of the outer frame portions is different than the angle of the taper of the connector 630 or the connector is not tapered.

Still referring to the example of FIGS. 75 and 76, the outer frame portions 620 can be configured to pinch together (e.g., in the manners described with respect to FIGS. 57 and 62-73). For example, the outer frame portions 620 can optionally be shapeset to provide a pinch point, the outer frame portions can be biased together by a spring or other resilient material, etc. In some implementations, the outer frame portions 620 can be shapeset such that one or more portions of each outer frame portion 620 is biased past a center line CL. When the two outer frame portions 620 are assembled in the device, the one or more portions 5700 engage one another. The optional biasing force past the center line CL creates a pinching force where the two outer frame portions 620 meet.

In some implementations, a discrete pinch point can be formed between the two outer frame portions 620 at or near the shoulders 5706 of the two outer frame portions 620 or the outer frame portions 620 can be shape set such that a plurality of pinch points are formed and/or such that one or more elongated pinch area is formed. In some implementations, the outer frame portions can be configured to provide an elongated pinch force along the outer frame portions from the connector 630 to the shoulders 5706.

Still referring to the example of FIGS. 75 and 76, in some implementations the outer frame portion 620 can optionally be configured to maximize the shoulder height C1. The outer frame portion 620 can optionally be configured to maximize the shoulder height C1 in any of the manners described herein.

The implantable valve repair device 600 can be configured to capture one or more leaflets. When multiple leaflets are captured, the leaflets can be brought or pinched together by the paddle frames 620. The paddle frames 620 can be configured to bring the leaflets together at the shoulder 5706. In some implementations, the outer frames 620 are configured to hold the leaflets together from the shoulder 5706 to the depth to which the leaflets extend in the device (e.g., to the hinge portion 138 of the clasp 130—see FIG. 76).

Still referring to the example of FIGS. 75 and 76, in some implementations, the outer frame portions 620 are configured such that the difference between the shoulder height in the wide position and the shoulder height in the narrow position is minimized. In some implementations, in the wide or substantially wide configuration, the leaflets 20, 22 are pressed together by the outer paddle frame portions 620 at a distance from the base or cap 640. In some implementations, the outer frame portions 620 can be moved from a completely widened configuration (i.e., 0% narrowed) where the outer paddle frame portions extend as far away from the inner paddle frame portions to a completely narrowed configuration (i.e., 100% narrowed) where the outer paddle frame portions are the same width or substantially the same width as the inner paddle frame portions.

In some implementations, the outer paddle frame portions 620 can be configured so that the shoulder height (such as when the paddle frames 1910 are between about 50% and 95% narrowed) and the shoulder height (such as when the paddle frames 1910 are 0% narrowed) are close together. For example, the shoulder height when the paddle frames are between 50% and 95% or any subrange narrowed can be between 70% and 100% or any subrange of the shoulder height that corresponds to the paddle frames being 0% narrowed.

The valve repair device can be configured in a wide variety of different ways to keep the shoulder height substantially constant. In some implementations, the outer frame portions 620 are shape set to keep the distance shoulder heights substantially the same and such that the outer frame portions 620 pinch together at the shoulder 5706.

Still referring to the example of FIGS. 75 and 76, in some implementations, the outer frame portions 620 can optionally wrap around the inner frame portions 610 when viewed from above (i.e., from the proximal end). In some implementations, wrapping the outer frame portions 620 around the inner frame portions 610 when viewed from above or the proximal end can help to constrain the paths of the outer surfaces (ventricular sides) of the valve leaflets 20, 22. For example, the valve leaflets 20, 22 are engaged by clasps 130 between sides 5902 of the inner frame portions 610. The sides 5902 of the inner frame portions 610 guide the valve leaflets 20, 22 from the clasps 130 toward one another.

In some implementations, when viewed from above or the proximal end, sides 5904 of the outer frame portions 620 are close to and optionally parallel to the sides 5902 of the inner frame portions 610. For example, the sides 5904 of the outer frame portions 620 can be a distance D3 (see FIG. 59) from the sides 5902 of the inner frame portions 610. In some implementations, the distance D3, when viewed from above or the proximal end, can be between 0.01 mm and 0.5 mm, such as between 0.01 mm and 0.2 mm, such as between 0.01 mm and 0.1 mm, or any subrange of these ranges. In some implementations, when viewed from above or the proximal end, the sides 5904 of the outer frame portions 620 are parallel to the sides 5902 of the inner frame portion.

In some implementations, when viewed from above or the proximal end, the outer frame portions 620 curves away from the inner frame portions 610 at curved portions 5908. In some implementations, the curved portions 5908 guide the leaflets 20, 22 both toward one another and toward sides of the outer frame portions. In some implementations, the curved portions 5908 constrain the paths of the outer surfaces (ventricular sides) of the valve leaflets 20, 22 to bring the leaflets together and/or inhibit bunching up or buckling of the leaflet.

In some implementations, the curved portions 5908 extend to a straight portion 5914 where are the leaflets 20, 22 are brought together when viewed from above or an atrial side. The straight portions 5914 extend from the curved portions 5908 to the sides or lateral extents 5910 of the outer frame portions 620.

In some implementations the outer frame portion 620 can be configured to have a fully narrowed width W3 (i.e., the width of the outer frame portion in the fully narrowed configuration) that is less than or equal to the width W4 of the inner frame portion 610, when viewed from the front or rear. In some implementations, the inner frame portions 610 do not change in width. FIG. 77 illustrates a partial view (i.e., about half of the device) from the front or rear of a device where outer frame portion 620 has a fully narrowed width W3 that is greater than the width W4 of the inner frame portion 610. FIG. 78 illustrates a partial view (i.e., about half of the device) from the front or rear of a device 600 where outer frame portion 620 has a fully narrowed width W3 that is equal to the width W4 of inner frame portion 610.

As is described in detail herein, the outer frame portions 620 can be moved between narrow and wide configurations. The narrow configuration allows the device 600 to be maneuvered through the patient's anatomy. For example, the narrowed outer frame portions 620 and attached paddles can be routed through the chordae tendineae CT to allow the paddles to be positioned on the outside or ventricular side of the leaflets 20, 22. By configuring the outer frame portions 620 to have a fully narrowed width W3 that is less than or equal to the width W4 of the inner frame portion 610, the device 600 can fit through any space, such as between the chordae tendineae CT, that the inner frame portion 610 can fit through.

The outer frame portions 620 can be configured to have a fully narrowed width W3 that is that is less than or equal to the width W4 of the inner frame portion 610 in a variety of different ways. For example, the outer frame portions 620 can be sized and shapeset such that the outer frame portion 620 has a fully narrowed width W3 that is less than or equal to the width W4 of the inner frame portion 610.

In some implementations, the outer frame portions 620 can be configured to have a fully narrowed width W3 that is less than or equal to the width W4 of the inner frame portion 610, while also being tapered (see FIG. 61), while being configured to wrap around the inner frame portions 610 when viewed from above or the proximal end (see FIGS. 59 and 60), being configured to pinch the leaflets together (see FIG. 57) and/or while maximizing the shoulder height C1 (see FIG. 58).

Some non-limiting examples of some of the concepts herein are recited below:

### EXAMPLES

Example 1. A device for repairing a native valve of a heart, the device comprising:

an anchor portion configured to connect to leaflets of the native valve, wherein the anchor portion comprises:

an outer paddle,

an inner paddle,

a clasp coupled to the inner paddle,

a paddle frame, and

a clip/connector coupling the paddle frame to at least one of the outer paddle, the inner paddle, and the clasp.

Example 2. The device of example 1, wherein the paddle frame comprises an outer frame portion and an inner frame portion.

Example 3. The device of example 2, wherein the clip/connector is coupled to at least one of the outer frame portion and the inner frame portion.

Example 4. The device of any of examples 1-3, wherein the clip/connector comprises an aperture configured to connect to the paddle frame.

Example 5. The device of example 4, wherein the aperture houses a portion of the paddle frame.

Example 6. The device of example 4, wherein the aperture houses at least one of the outer frame portion and the inner frame portion of the paddle frame.

Example 7. The device of any of examples 4-6, wherein the aperture comprises a square shaped cross section.

Example 8. The device of any of examples 4-6, wherein the aperture comprises a cross sectional shape that is the same as the cross-sectional shape as at least one of the outer frame portion and the inner frame portion.

Example 9. The device of any of examples 1-3, wherein the clip/connector comprises a first aperture and a second aperture.

Example 10. The device of example 9, wherein the first aperture is coupled to the inner paddle portion and the second aperture is coupled to the outer paddle portion.

Example 11. The device of any of examples 4-10, wherein the clip/connector comprises an open configuration and a closed configuration.

Example 12. The device of example 11, wherein in the open position, the head portion is open such that the aperture is accessible by way of a channel, and wherein in the closed configuration, the head portion is closed such that the aperture is not accessible.

Example 13. The device of example 12, wherein at least one of the inner frame portion and the outer frame portion can be disposed through the channel and into the aperture.

Example 14. The device of any of the preceding examples, wherein the clip/connector comprises a head portion and a stem portion.

Example 15. The device of example 14, wherein the head portion comprises one or engaging members.

Example 16. The device of example 14, wherein the one or more engaging members engage at least one of the inner paddle and outer paddle.

Example 17. The device of example 14, wherein the one or more engaging members are positioned at a distal end of the clip/connector.

Example 18. The device of any of examples 14-17, wherein the engaging members comprise a zig zag configuration.

Example 19. The device of any of examples 9-18, wherein the engaging members extend through and hook onto at least one of the outer paddle and the inner paddle.

Example 20. The device of any of the preceding examples, wherein the clip/connector is a first paddle frame clip/connector and the device further comprises a second paddle frame clip/connector, wherein the first paddle frame clip/connector is coupled to the outer paddle and the second paddle frame clip/connector is coupled to the inner paddle.

Example 21. A system comprising:

a device for repairing a native valve of a heart, the device comprising:
an anchor portion configured to connect to leaflets of the native valve, wherein the anchor portion comprises:
an outer paddle,
an inner paddle,
a clasp coupled to the inner paddle,
a paddle frame, and
a paddle frame clip/connector coupling the paddle frame to at least one of the outer paddle, inner paddle, or the clasp, and

a catheter coupled to the device.

Example 22. The system of example 1, wherein the paddle frame comprises an outer frame portion and an inner frame portion.

Example 23. The system of example 22, wherein the paddle frame clip/connector is coupled to at least one of the outer frame portion and the inner frame portion.

Example 24. The system of any of examples 1-23, wherein the paddle frame clip/connector comprises an aperture.

Example 25. The system of example 24, wherein the aperture houses a portion of the paddle frame.

Example 26. The system of example 24, wherein the aperture houses at least one of the outer frame portion and the inner frame portion of the paddle frame.

Example 27. The system of any of examples 24-26, wherein the aperture comprises a square shaped cross section.

Example 28. The system of any of examples 24-26, wherein the aperture comprises a cross sectional shape that is the same as the cross-sectional shape as at least one of the outer frame portion and the inner frame portion.

Example 29. The system of any of examples 21-23, wherein the paddle frame clip/connector comprises a first aperture and a second aperture.

Example 30. The system of example 29, wherein the first aperture is coupled to the inner paddle frame portion and the second aperture is coupled to the outer paddle frame portion.

Example 31. The system of any of examples 24-30, wherein the paddle frame clip/connector comprises an open configuration and a closed configuration.

Example 32. The system of example 31, wherein in the open configuration, the head portion is open such that the aperture is accessible by way of a channel, and wherein in the closed configuration, the head portion is closed such that the aperture is not accessible.

Example 33. The system of example 32, wherein at least one of the inner frame portion and the outer frame portion can be disposed through the channel and into the aperture.

Example 34. The system of any of the preceding examples, wherein the paddle frame clip/connector comprises a head portion and a stem portion.

Example 35. The system of example 34, wherein the head portion comprises one or more engaging members.

Example 36. The system of example 34, wherein the one or more engaging members engage at least one of the inner paddle and outer paddle.

Example 37. The system of example 34, wherein the one or more engaging members are positioned at a distal end of the paddle frame clip/connector.

Example 38. The system of any of examples 34-37, wherein the engaging members comprise a zig zag configuration.

Example 39. The system of any of examples 29-38, wherein the engaging members extend through and hook onto at least one of the outer paddle and inner paddle.

Example 40. The system of any of the preceding examples, wherein the device comprises a first paddle frame clip/connector and a second paddle frame clip/connector, wherein the first paddle frame clip/connector is coupled to the outer paddle and the second paddle frame clip/connector is coupled to the inner paddle.

Example 41. A device for repairing a native valve of a heart, the device comprising:

an anchor portion configured to connect to leaflets of the native valve, wherein the anchor portion comprises:

an outer paddle,

an inner paddle,

a clasp coupled to the inner paddle,

a paddle frame comprising an outer frame portion, and

wherein the outer frame portion comprises a flex limiting portion, wherein the flex limiting portion comprises a clip/connector region disposed between a first flex limiting component and a second flex limiting component.

Example 42. The device of example 41, wherein the at least one of the first flex limiting component and the second flex limiting component comprises an L-shaped bend.

Example 43. The device of example 42, wherein the clip/connector region comprises a rectangular, square, trapezoidal, or an irregularly shaped cross section.

Example 44. The device of example 43, further comprising a paddle frame clip/connector coupled with the clip/connector region between the first flex limiting component and the second flex limiting component.

Example 45. The device of example 44, wherein the clip/connector region comprises a width that is substantially the same as a width of the paddle frame clip/connector.

Example 46. The device of any of examples 44-45, wherein the clip/connector region comprises a cross sectional shape that matches the cross-sectional shape of the paddle frame clip/connector.

Example 47. The device of any of examples 41-46, wherein the clip/connector region comprises a plurality of segments between a surface of the clip/connector region.

Example 48. The device of example 47, wherein each of the plurality of segments is angled between 45 degrees and 90 degreed relative to the surface.

Example 49. The device of any of examples 41-48, wherein the outer frame portion comprises a first curve and a second curve, wherein a radius of the first curve is less than a radius of the second curve.

Example 50. The device of any of examples 41-49, wherein the outer frame portion comprises an expanded state and a narrowed state.

Example 51. A system comprising:

a device for repairing a native valve of a heart, the device comprising:
an anchor portion configured to connect to leaflets of the native valve, wherein the anchor portion comprises:
an outer paddle,
an inner paddle,
a clasp coupled to the inner paddle, and
a paddle frame comprising an outer frame portion,
wherein the outer frame portion comprises a flex limiting portion, wherein the flex limiting portion comprises a clip/connector region disposed between a first flex limiting component and a second flex limiting component; and

a catheter coupled with the device.

Example 52. The system of example 51, wherein the at least one of the first flex limiting component and the second flex limiting component comprises an L-shaped bend.

Example 53. The system of example 52, wherein the clip/connector region comprises a rectangular, square, trapezoidal, or an irregularly shaped cross section.

Example 54. The system of example 53, further comprising a paddle frame clip/connector coupled with the clip/connector region between the first flex limiting component and the second flex limiting component.

Example 55. The system of example 54, wherein the clip/connector region comprises a width that is substantially the same as a width of the paddle frame clip/connector.

Example 56. The system of any of examples 54-55, wherein the clip/connector region comprises a cross sectional shape that matches the cross-sectional shape of the paddle frame clip/connector.

Example 57. The system of any of examples 51-56, wherein the clip/connector region comprises a plurality of segments between a surface of the clip/connector region.

Example 58. The system of example 57, wherein each of the plurality of segments is angled between 45 degrees and 90 degreed relative to the surface.

Example 59. The system of any of examples 51-58, wherein the outer frame portion comprises a first curve and a second curve, wherein a radius of the first curve is less than a radius of the second curve.

Example 60. The system of any of examples 51-59, wherein the outer frame portion comprises an expanded state and a narrowed state.

Example 61. A device for repairing a native valve of a heart, the device comprising:

a pair of paddle frames that are configured to move between a wide configuration and a narrow configuration;

wherein the pair of paddle frames are moveable between an open position and a closed position where the paddle frames press leaflets of the native heart valve together;

wherein the pair of paddle frames have a first paddle frame coaptation position where the native leaflets are first pressed together when the pair of paddle frames are in the closed position and are in the wide configuration;

wherein the pair of paddle frames have a second paddle frame coaptation position where the native leaflets are first pressed together when the pair of paddle frames are in the closed position and are in a fifty percent narrowed configuration;

wherein the second paddle frame coaptation position is within 2 mm of the first paddle frame coaptation position.

Example 62. The device of example 61 wherein the second paddle frame coaptation position is within 1 mm of the first battle frame coaptation position.

Example 63. The device of example 61 where in the second paddle frame coaptation position is within 0.75 mm of the first paddle frame coaptation position.

Example 64. The device of example 61 where in the second paddle frame coaptation position is within 0.5 mm of the first paddle frame coaptation position.

Example 65. The device of example 61, wherein the second paddle frame coaptation position is between 0.25 mm and 0.75 mm of the first paddle frame coaptation position.

Example 66. A device for repairing a native valve of a heart, the device comprising:

a pair of paddle frames comprising:
a pair of inner paddle frame portions;
a pair of adjustable width outer paddle frame portions;

wherein the pair of paddle frames are movable between an open position and a closed position; and

wherein the outer frame portions are configured to pinch together in the closed position.

Example 67. The device of example 66 wherein the pair of inner frame portions do not pinch together in the closed position.

Example 68. The device of any one of examples 66-67 wherein the pair of outer frame portions are shapeset to provide a pinch point.

Example 69. The device of any one of examples 66-68 wherein the pair of outer frame portions are shapeset such that one or more portions of each of the pair of outer frame portions is biased past a center line CL of the device.

Example 70. A system comprising:

a device for repairing a native valve of a heart, the device comprising:
a pair of paddle frames comprising:
a pair of inner paddle frame portions;
a pair of adjustable width outer paddle frame portions;
wherein the pair of paddle frames are movable between an open position and a closed position;
wherein the outer frame portions are configured to pinch together in the closed position; and

a catheter coupled with the device.

Example 71. The system of example 70 wherein the pair of inner frame portions do not pinch together in the closed position.

Example 72. The system of any one of examples 70-71 wherein the pair of outer frame portions are shapeset to provide a pinch point.

Example 73. The system of any one of examples 70-72 wherein the pair of outer frame portions are shapeset such that one or more portions of each of the pair of outer frame portions is biased past a center line CL of the device.

Example 74. A device for repairing a native valve of a heart, the device comprising:

a pair of paddle frames;

wherein the pair of paddle frames are movable between a fully widened configuration and a fully narrowed configuration;

wherein the pair of paddle frames are movable between an open position and a closed position both in the fully widened configuration and in the fully narrowed configuration;

wherein the pair of paddle frames have a first shoulder height when the pair of paddle frames are closed and are in the fully narrowed configuration;

wherein the pair of paddle frames have a second shoulder height when the pair of paddle frames are closed and are in the fully widened configuration;

wherein the first shoulder height is between 70% and 100% of the second shoulder height.

Example 75. The device of example 74 wherein the outer frame portions are configured to pinch together in the closed position in both the fully narrowed configuration and the fully widened configuration.

Example 76. The device of any one of examples 74-75 wherein each paddle frame of the pair of paddle frames comprises a fixed width inner frame portion and an adjustable width outer frame portion.

Example 77. The device of example 76 wherein the adjustable width outer frame portion is movable between the fully narrowed configuration and fully widened configuration.

Example 78. A system comprising:

a device for repairing a native valve of a heart, the device comprising:

a pair of paddle frames;

wherein the pair of paddle frames are movable between a fully widened configuration and a fully narrowed configuration;

wherein the pair of paddle frames are movable between an open position and a closed position both in the fully widened configuration and in the fully narrowed configuration;

wherein the pair of paddle frames have a first shoulder height when the pair of paddle frames are closed and are in the fully narrowed configuration;

wherein the pair of paddle frames have a second shoulder height when the pair of paddle frames are closed and are in the fully widened configuration;

wherein the first shoulder height is between 70% and 100% of the second shoulder height; and

a catheter coupled with the device.

Example 79. The system of example 78 wherein the outer frame portions are configured to pinch together in the closed position in both the fully narrowed configuration and the fully widened configuration.

Example 80. The system of any one of examples 78-79 wherein each paddle frame of the pair of paddle frames comprises a fixed width inner frame portion and an adjustable width outer frame portion.

Example 81. The system of example 80 wherein the adjustable width outer frame portion is movable between the fully narrowed configuration and fully widened configuration.

Example 82. A device for repairing a native valve of a heart, the device comprising:

a pair of paddle frames;

wherein the pair of paddle frames are movable between a fully widened configuration and a fully narrowed configuration;

wherein the pair of paddle frames are movable between an open position and a closed position both in the fully widened configuration and in the fully narrowed configuration;

wherein the pair of paddle frames have a first shoulder height when the pair of paddle frames are closed and are in the fully narrowed configuration;

wherein the pair of paddle frames have a second shoulder height when the pair of paddle frames are closed and are in the fully widened configuration;

wherein the first shoulder height is within 1 mm of the second shoulder height.

Example 83. The device of example 82 wherein the outer frame portions are configured to pinch together in the closed position in both the fully narrowed configuration and the fully widened configuration.

Example 84. The device of example 82 wherein each paddle frame of the pair of paddle frames comprises a fixed width inner frame portion and an adjustable width outer frame portion.

Example 85. The device of example 84 wherein the adjustable width outer frame portion is movable between the fully narrowed configuration and fully widened configuration.

Example 86. A system comprising:

a device for repairing a native valve of a heart, the device comprising:

a pair of paddle frames;

wherein the pair of paddle frames are movable between a fully widened configuration and a fully narrowed configuration;

wherein the pair of paddle frames are movable between an open position and a closed position both in the fully widened configuration and in the fully narrowed configuration;

wherein the pair of paddle frames have a first shoulder height when the pair of paddle frames are closed and are in the fully narrowed configuration;

wherein the pair of paddle frames have a second shoulder height when the pair of paddle frames are closed and are in the fully widened configuration;

wherein the first shoulder height is within 1 mm of the second shoulder height; and

a catheter coupled with the device.

Example 87. The system of example 86 wherein the outer frame portions are configured to pinch together in the closed position in both the fully narrowed configuration and the fully widened configuration.

Example 88. The system of any one of examples 86-87 wherein each paddle frame of the pair of paddle frames comprises a fixed width inner frame portion and an adjustable width outer frame portion.

Example 89. The system of example 88 wherein the adjustable width outer frame portion is movable between the fully narrowed configuration and fully widened configuration.

Example 90. A device for repairing a native valve of a heart, the device comprising:

a pair of paddle frames comprising:
a pair of inner paddle frame portions;
a pair of adjustable width outer paddle frame portions;

wherein the pair of paddle frames are movable between a fully widened configuration and a fully narrowed configuration;

wherein the pair of paddle frames are movable between an open position and a closed position both in the fully widened configuration and in the fully narrowed configuration; and

wherein each outer paddle frame portion of the pair of paddle frame portions wraps around a corresponding inner paddle frame portion of the pair of inner paddle frame portions when viewed from a proximal end, the pair of paddles are in the closed position, and the pair of outer paddle frame portions are in the fully widened configuration.

Example 91. The device of example 90 further comprising a pair of clasps disposed between the pair of inner paddle frame portions.

Example 92. The device of example 91 wherein each clasp of the pair of clasps is disposed between sides of the inner paddle frame portions when viewed from a proximal side.

Example 93. The device of any one of examples 90-92 wherein sides of the outer frame portions are parallel to sides of the inner frame portions when viewed from a proximal side.

Example 94. The device of any one of examples 90-93 wherein a distance between sides of the outer frame portions and the inner frame portions is between 0.01 mm and 0.5 mm when viewed from a proximal side.

Example 95. The device of any one of examples 92-94 wherein the outer frame portions curve away from the inner frame portions within 0.5 mm of ends of the sides of the inner frame portion when viewed from the proximal end.

Example 96. A system comprising:

a device for repairing a native valve of a heart, the device comprising:
a pair of paddle frames comprising:
   a pair of inner paddle frame portions;
   a pair of adjustable width outer paddle frame portions;
wherein the pair of paddle frames are movable between a fully widened configuration and a fully narrowed configuration;
wherein the pair of paddle frames are movable between an open position and a closed position both in the fully widened configuration and in the fully narrowed configuration;
wherein each outer paddle frame portion of the pair of paddle frame portions wraps around a corresponding inner paddle frame portion of the pair of inner paddle frame portions when viewed from a proximal end, the pair of paddles are in the closed position, and the pair of outer paddle frame portions are in the fully widened configuration; and

a catheter coupled with the device.

Example 97. The system of example 96 further comprising a pair of clasps disposed between the pair of inner paddle frame portions.

Example 98. The system of example 97 wherein each clasp of the pair of clasps is disposed between sides of the inner paddle frame portions when viewed from a proximal side.

Example 99. The system of any one of examples 96-98 wherein sides of the outer frame portions are parallel to sides of the inner frame portions when viewed from a proximal side.

Example 100. The system of any one of examples 96-99 wherein a distance between sides of the outer frame portions and the inner frame portions is between 0.01 mm and 0.5 mm when viewed from a proximal side.

Example 101. The system of any one of examples 98-100 wherein the outer frame portions curve away from the inner frame portions within 0.5 mm of ends of the sides of the inner frame portion when viewed from the proximal end.

Example 102. A device for repairing a native valve of a heart, the device comprising:

a pair of paddle frames;

wherein the pair of paddle frames are movable between a fully widened configuration and a fully narrowed configuration;

wherein the pair of paddle frames are movable between an open position and a closed position both in the fully widened configuration and in the fully narrowed configuration; and

wherein the pair of paddle frames are tapered in a direction from a proximal end toward a distal end when viewed from the front, when the pair of paddle frames are closed, and when the pair of paddle frames are in the fully narrowed configuration.

Example 103. The device of example 102 wherein the pair of paddle frames are tapered at an angle between 30 and 80 degrees.

Example 104. The device of any one of examples 102 to 103 further comprising a connector that extends from the pair of paddle frames to a cap of the device.

Example 105. The device of example 104 wherein the connector is tapered in the direction from the proximal end toward the distal end.

Example 106. The device of example 105 wherein a taper angle of the connector is within 5 degrees of the angle of taper of the pair of paddle frames.

Example 107. The device of example 105 wherein a taper of the connector is continuous with the taper of the pair of paddle frames.

Example 108. A system comprising:

a valve repair device comprising:
a pair of paddle frames;

wherein the pair of paddle frames are movable between a fully widened configuration and a fully narrowed configuration;

wherein the pair of paddle frames are movable between an open position and a closed position both in the fully widened configuration and in the fully narrowed configuration;

wherein the pair of paddle frames are tapered in a direction from a proximal end toward a distal end when viewed from the front, when the pair of paddle frames are closed, and when the pair of paddle frames are in the fully narrowed configuration; and a catheter coupled with the device.

Example 109. The system of example 108 wherein the pair of paddle frames are tapered at an angle between 30 and 80 degrees.

Example 110. The system of any one of examples 108 to 109 further comprising a connector that extends from the pair of paddle frames to a cap of the device.

Example 111. The system of example 110 wherein the connector is tapered in the direction from the proximal end toward the distal end.

Example 112. The system of example 111 wherein a taper angle of the connector is within 5 degrees of the angle of taper of the pair of paddle frames.

Example 113. The system of example 111 wherein a taper of the connector is continuous with the taper of the pair of paddle frames.

Example 114. A device for repairing a native valve of a heart, the device comprising:

a pair of paddle frames comprising:
a pair of inner paddle frame portions having a first width;
a pair of adjustable width outer paddle frame portions;

wherein the pair of adjustable width paddle frame portions are movable between a fully widened configuration and a fully narrowed configuration having a second width;

wherein the pair of paddle frames are movable between an open position and a closed position; and

wherein the second width is less than or equal to the first width.

Example 115. The device of example 114 wherein the pair of adjustable width paddle frame portions wrap around the pair of inner paddle frame portions when viewed from a proximal end of the device.

Example 116. The device of any one of examples 114 to 115 wherein the pair of adjustable width paddle frame portions are configured to pinch together when the pair of adjustable width paddle fame portions are in the closed position.

Example 117. A system comprising:

a valve repair device comprising:
a pair of paddle frames comprising:
   a pair of inner paddle frame portions having a first width;
   a pair of adjustable width outer paddle frame portions;

wherein the pair of adjustable width paddle frame portions are movable between a fully widened configuration and a fully narrowed configuration having a second width;

wherein the pair of paddle frames are movable between an open position and a closed position;

wherein the second width is less than or equal to the first width;

a catheter coupled with the device.

Example 118. The system of example 117 wherein the pair of adjustable width paddle frame portions wrap around the pair of inner paddle frame portions when viewed from a proximal end of the device.

Example 119. The system of any one of examples 117 to 118 wherein the pair of adjustable width paddle frame portions are configured to pinch together when the pair of adjustable width paddle fame portions are in the closed position.

Example 120. Any of examples 1-119 used or performed on a living animal or on a non-living simulation, such as on a cadaver, cadaver heart, anthropomorphic ghost, simulator (e.g., with the body parts, tissue, etc. being simulated), etc.

Example 121. Any of examples 1-121 where one or more components of the device or system is sterilized.

The treatment techniques, methods, steps, devices, systems etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

While various inventive aspects, concepts and features of the disclosures can be described and illustrated herein as embodied in combination in the examples herein, these various aspects, concepts, and features can be used in many alternative examples, either individually or in various combinations and sub-combinations thereof. Unless expressly excluded herein all such combinations and sub-combinations are intended to be within the scope of the present application. Still further, while various alternative examples as to the various aspects, concepts, and features of the disclosures-such as alternative materials, structures, configurations, methods, devices, and components, alternatives as to form, fit, and function, and so on—can be described herein, such descriptions are not intended to be a complete or exhaustive list of available alternative examples, whether presently known or later developed. Those skilled in the art can readily adopt one or more of the inventive aspects, concepts, or features into additional examples and uses within the scope of the present application even if such examples are not expressly disclosed herein.

Additionally, even though some features, concepts, or aspects of the disclosures can be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description of the examples in the specification.

What is claimed is:

1. A device for repairing a native valve of a heart, the device comprising:
an anchor portion configured to connect to leaflets of the native valve, wherein the anchor portion comprises:
an outer paddle,
an inner paddle,
a clasp coupled to the inner paddle,
a paddle frame,
a clip coupling the paddle frame to the outer paddle,
wherein the clip extends along the outer paddle; and
wherein at least a portion of the clip is disposed between the inner paddle and the outer paddle when the anchor portion is in a closed configuration.

2. The device of claim 1, wherein the paddle frame comprises an expandable outer frame portion and an inner frame portion.

3. The device of claim 2, wherein the outer frame portion comprises a flex limiting portion, wherein the flex limiting portion comprises a clip region disposed between a first flex limiting component and a second flex limiting component.

4. The device of claim 3, wherein at least one of the first flex limiting component and the second flex limiting component comprises an L-shaped bend.

5. The device of claim 2, wherein the outer frame portion comprises a flex limiting portion, wherein the flex limiting portion includes one or more openings or holes in the expandable outer frame portion.

6. The device of claim 2, wherein the paddle frame is movable between a fully widened configuration and a fully narrowed configuration;
wherein the paddle frame is movable between an open position and a closed position both in the fully widened configuration and in the fully narrowed configuration;
wherein the paddle frame has a first shoulder height when the paddle frame is closed and is in the fully narrowed configuration;
wherein the paddle frame has a second shoulder height when the paddle frame is closed and is in the fully widened configuration; and
wherein the first shoulder height is between 70% and 100% of the second shoulder height.

7. The device of claim 6, wherein the paddle frame is tapered in a direction from a proximal end toward a distal end when viewed from the front, when the paddle frame is closed and when the paddle frame is in the fully narrowed configuration.

8. The device of claim 7, wherein the paddle frame is tapered at an angle between 30 and 80 degrees.

9. The device of claim 1, further comprising a second clip that extends along and reinforces the inner paddle.

10. A system comprising:
a device for repairing a native valve of a heart, the device comprising:
an anchor portion configured to connect to leaflets of the native valve, wherein the anchor portion comprises:
an outer paddle,
an inner paddle,
a clasp coupled to the inner paddle,
a paddle frame,
a clip coupling the paddle frame to the outer paddle,
wherein the clip extends along the outer paddle; and
wherein at least a portion of the clip is disposed between the inner paddle and the outer paddle when the anchor portion is in a closed configuration.

11. The system of claim 10, wherein the paddle frame comprises an expandable outer frame portion and an inner frame portion.

12. The system of claim 11, wherein the outer frame portion comprises a flex limiting portion, wherein the flex limiting portion comprises a clip region disposed between a first flex limiting component and a second flex limiting component.

13. The system of claim 12, wherein at least one of the first flex limiting component and the second flex limiting component comprises an L-shaped bend.

14. The system of claim 11, wherein the outer frame portion comprises a flex limiting portion, wherein the flex limiting portion includes one or more openings or holes in the expandable outer frame portion.

15. The system of claim 11, wherein the paddle frame is movable between a fully widened configuration and a fully narrowed configuration;
wherein the paddle frame is movable between an open position and a closed position both in the fully widened configuration and in the fully narrowed configuration;
wherein the paddle frame has a first shoulder height when the paddle frame is closed and are is in the fully narrowed configuration;
wherein the paddle frame has a second shoulder height when the paddle frame is closed and is in the fully widened configuration;
wherein the first shoulder height is between 70% and 100% of the second shoulder height.

16. The system of claim 15, wherein the paddle frame is tapered in a direction from a proximal end toward a distal end when viewed from the front, when the paddle frame is closed and when the paddle frame is in the fully narrowed configuration.

17. The system of claim 15, wherein the paddle frame is tapered at an angle between 30 and 80 degrees.

18. The system of claim 10, further comprising a second clip that extends along and reinforces the inner paddle.

19. A device for repairing a native valve of a heart, the device comprising:
an anchor portion configured to connect to leaflets of the native valve, wherein the anchor portion comprises:
an outer paddle,
an inner paddle,
a clasp coupled to the inner paddle, and
a paddle frame comprising a flex limiting portion, wherein the flex limiting portion comprises a connector region disposed between a first flex limiting component and a second flex limiting component.

20. The device of claim 19, wherein the connector region comprises a rectangular, square, trapezoidal, or an irregularly shaped cross section.
